SEQUENCE LISTING (92 sequences)

SEQ ID NO: 1 - protein, 118 aa, Artificial Sequence
  PreS1 HBV antigen
GGWSSKPRKGMGTNLSVPNPLGFFPDHQLDPAFGANSNNPDWDFNPNKDHWPDANKVGAGAFGPGFTPPHGGLLGWSPQAQGILTTVPAAPPPASTNRQSGRQPTPLSPPLRDTHPQA

SEQ ID NO: 2 - DNA, 357 bp, Artificial Sequence
  PreS1 HBV antigen coding sequence
ggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataa

SEQ ID NO: 3 - protein, 100 aa, Artificial Sequence
  D1-19 PreS1 HBV antigen
NPLGFFPDHQLDPAFGANSNNPDWDFNPNKDHWPDANKVGAGAFGPGFTPPHGGLLGWSPQAQGILTTVPAAPPPASTNRQSGRQPTPLSPPLRDTHPQA

SEQ ID NO: 4 - DNA, 303 bp, Artificial Sequence
  D1-19 PreS1 HBV antigen coding sequence
aatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataa

SEQ ID NO: 5 - protein, 281 aa, Artificial Sequence
  PreS2.S HBV antigen
MQWNSTTFHQTLQDPRVRGLYFPAGGSSSGTVNPVPTTASPISSIFSRTGDPAPNMENITSGFLGPLLVLQAGFFLLTRILTIPQSLDSWWTSLNFLGGTPVCLGQNSQSPTSNHSPTSCPPICPGYRWMCLRRFIIFLFILLLCLIFLLVLLDYQGMLPVCPLIPGSSTTSTGPCKTCTIPAQGTSMFPSCCCTKPSDGNCTCIPIPSSWAFAKYLWEWASVRFSWLSLLVPFVQWFVGLSPTVWLSVIWMMWYWGPSLYNILSPFLPLLPIFFCLWVYI

SEQ ID NO: 6 - DNA, 846 bp, Artificial Sequence
  PreS2.S HBV antigen coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaa

SEQ ID NO: 7 - protein, 148 aa, Artificial Sequence
  HBV core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 8 - DNA, 444 bp, Artificial Sequence
  HBV core antigen coding sequence
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 9 - protein, 843 aa, Artificial Sequence
  HBV polymerase antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSKRHGDESFCSQSSGILSRSPVGPCIQSQLRKSRLGLQPQQGHLARRQQGRSGSIRARVHPTTRRTFGVEPSGSGHIDNSASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 10 - DNA, 2529 bp, Artificial Sequence
  HBV polymerase antigen coding sequence
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 11 - protein, 22 aa, Artificial Sequence
  P2A autoprotease
GSGATNFSLLKQAGDVEENPGP

SEQ ID NO: 12 - DNA, 66 bp, Artificial Sequence
  P2A autoprotease coding sequence
ggaagcggagctactaacttcagcctgctgaagcaggctggagacgtggaggagaaccctggacct

SEQ ID NO: 13 - DNA, 587 bp, Artificial Sequence
  EMCV IRES
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 14 - DNA, 747 bp, Artificial Sequence
  EV71 IRES
ttaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgacc

SEQ ID NO: 15 - DNA, 3174 bp, Artificial Sequence
  Core-2A-Pol coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 16 - DNA, 3174 bp, Artificial Sequence
  Pol-2A-Core coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 17 - DNA, 3698 bp, Artificial Sequence
  Core coding sequence-IRES(EMCV)-Pol coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 18 - DNA, 3698 bp, Artificial Sequence
  Pol coding sequence-IRES (EMCV)-Core coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 19 - DNA, 3858 bp, Artificial Sequence
  Core coding sequence-IRES (EV71)-Pol coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 20 - DNA, 3858 bp, Artificial Sequence
  Pol coding sequence -IRES (EV71)-Core coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 21 - DNA, 1326 bp, Artificial Sequence
  PreS2.S-2A-preS1 coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 22 - DNA, 1326 bp, Artificial Sequence
  preS1-2A-PreS2.S coding sequence
atggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggaagcggagctactaacttcagcctgctgaagcaggctggagacgtggaggagaaccctggacctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 23 - DNA, 1850 bp, Artificial Sequence
  PreS2.S coding sequence -IRES (EMCV)-preS1 coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 24 - DNA, 1850 bp, Artificial Sequence
  preS1 coding sequence -IRES (EMCV)-PreS2.S coding sequence
atggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 25 - DNA, 2010 bp, Artificial Sequence
  PreS2.S coding sequence -IRES (EV71)-preS1 coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 26 - DNA, 2010 bp, Artificial Sequence
  preS1 coding sequence -IRES (EV71)-PreS2.S coding sequence
atggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 27 - DNA, 4566 bp, Artificial Sequence
  Core-2A-Pol-2A-PreS.S-2A-preS1 coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 28 - DNA, 4566 bp, Artificial Sequence
  Pol-2A-Core-2A-PreS2.S-2A-preS1 coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggatcaggcgctacgaattttagccttctgaagcaagcgggagacgttgaagaaaacccagggcctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 29 - DNA, 4566 bp, Artificial Sequence
  PreS2.S-2A-preS1-2A-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggatcaggcgctacgaattttagccttctgaagcaagcgggagacgttgaagaaaacccagggcctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtgggtccggagcgactaacttttccctgctgaaacaagcgggtgacgtcgaagagaatccgggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 30 - DNA, 4566 bp, Artificial Sequence
  PreS2.S-2A-preS1-2A-Pol-2A-Core coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccagggtccggagcgactaacttttccctgctgaaacaagcgggtgacgtcgaagagaatccgggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 31 - DNA, 5090 bp, Artificial Sequence
  Core-2A-Pol coding sequence -IRES (EMCV)-PreS2.S-2A-preS1 coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 32 - DNA, 5090 bp, Artificial Sequence
  PreS2.S-2A-preS1 coding sequence -IRES (EMCV)-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtgggtccggagcgactaacttttccctgctgaaacaagcgggtgacgtcgaagagaatccgggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 33 - DNA, 5250 bp, Artificial Sequence
  Core-2A-Pol coding sequence -IRES (EV71)-PreS2.S-2A-preS1 coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 34 - DNA, 5250 bp, Artificial Sequence
  PreS2.S-2A-preS1 coding sequence -IRES (EV71)-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtgggtccggagcgactaacttttccctgctgaaacaagcgggtgacgtcgaagagaatccgggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 35 - DNA, 5090 bp, Artificial Sequence
  Pol-2A-Core coding sequence -IRES (EMCV)-PreS2.S-2A-preS1 coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 36 - DNA, 5250 bp, Artificial Sequence
  Pol-2A-Core coding sequence -IRES (EV71)-PreS2.S-2A-preS1 coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 37 - DNA, 5090 bp, Artificial Sequence
  PreS2.S-2A-preS1 coding sequence -IRES (EMCV)-Pol-2A-Core coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccagggtccggagcgactaacttttccctgctgaaacaagcgggtgacgtcgaagagaatccgggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 38 - DNA, 5250 bp, Artificial Sequence
  PreS2.S-2A-preS1 coding sequence -IRES (EV71)-Pol-2A-Core coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccagggtccggagcgactaacttttccctgctgaaacaagcgggtgacgtcgaagagaatccgggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 39 - DNA, 4083 bp, Artificial Sequence
  Core-2A-Pol-2A-PreS2.S coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 40 - DNA, 4083 bp, Artificial Sequence
  Pol-2A-Core-2A-PreS2.S coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggatcaggcgctacgaattttagccttctgaagcaagcgggagacgttgaagaaaacccagggcctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 41 - DNA, 4083 bp, Artificial Sequence
  PreS2.S-2A-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 42 - DNA, 4083 bp, Artificial Sequence
  PreS2.S-2A-Pol-2A-Core coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 43 - DNA, 4607 bp, Artificial Sequence
  Core-2A-Pol coding sequence -IRES (EMCV)-PreS2.S coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 44 - DNA, 4607 bp, Artificial Sequence
  PreS2.S coding sequence -IRES (EMCV)-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 45 - DNA, 4767 bp, Artificial Sequence
  Core-2A-Pol coding sequence -IRES (EV71)-PreS2.S coding sequence
atggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 46 - DNA, 4767 bp, Artificial Sequence
  PreS2.S coding sequence -IRES (EV71)-Core-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 47 - DNA, 4607 bp, Artificial Sequence
  Pol-2A-Core coding sequence -IRES (EMCV)-PreS2.S coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 48 - DNA, 4767 bp, Artificial Sequence
  Pol-2A-Core coding sequence -IRES (EV71)-PreS2.S coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatt

SEQ ID NO: 49 - DNA, 4607 bp, Artificial Sequence
  PreS2.S-IRES (EMCV)-Pol-2A-Core
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 50 - DNA, 4767 bp, Artificial Sequence
  PreS2.S coding sequence -IRES (EV71)-Pol-2A-Core coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgt

SEQ ID NO: 51 - DNA, 3987 bp, Artificial Sequence
  Pol-2A-PreS2.S-2A-preS1 coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggatcaggcgctacgaattttagccttctgaagcaagcgggagacgttgaagaaaacccagggcctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataa

SEQ ID NO: 52 - DNA, 3987 bp, Artificial Sequence
  PreS2.S-2A-preS1-2A-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataa

SEQ ID NO: 53 - DNA, 4508 bp, Artificial Sequence
  PreS2.S-2A-preS1 coding sequence -IRES (EMCV)-Pol coding sequence
atgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 54 - DNA, 4508 bp, Artificial Sequence
  Pol coding sequence -IRES (EMCV)-PreS2.S-2A-preS1 coding sequence
atggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggca

SEQ ID NO: 55 - DNA, 44 bp, Artificial Sequence
  5′ UTR
ataggcggcgcatgagagaagcccagaccaattacctacccaaa

SEQ ID NO: 56 - DNA, 195 bp, Artificial Sequence
  5′ replication sequence
taggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattgga

SEQ ID NO: 57 - DNA, 142 bp, Artificial Sequence
  DLP capsid enhancer
atagtcagcatagtacatttcatctgactaatactacaacaccaccaccatgaatagaggattctttaacatgctcggccgccgccccttcccggcccccactgccatgtggaggccgcggagaaggaggcaggcggccccg

SEQ ID NO: 58 - DNA, 1602 bp, Artificial Sequence
  nsp1 coding sequence
gagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 59 - DNA, 2382 bp, Artificial Sequence
  nsp2 coding sequence
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 60 - DNA, 1671 bp, Artificial Sequence
  nsp3 coding sequence
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 61 - DNA, 1821 bp, Artificial Sequence
  nsp4 coding sequence
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 62 - DNA, 24 bp, Artificial Sequence
  26S subgenomic promoter
ctctctacggctaacctgaatgga

SEQ ID NO: 63 - DNA, 117 bp, Artificial Sequence
  3′ UTR
atacagcagcaattggcaagctgcttacatagaactcgcggcgattggcatgccgctttaaaatttttattttatttttcttttcttttccgaatcggattttgtttttaatatttc

SEQ ID NO: 64 - DNA, 40 bp, Artificial Sequence
  Poly Adenosine
aaaaaaaaaaaaaaaaaaaaaaaaaaaaaaaaaaaaaaaa

SEQ ID NO: 65 - DNA, 11338 bp, Artificial Sequence
  Core-2A-Pol Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtggaagcggagctactaacttcagcctgctgaagcaggctggagacgtggaggagaaccctggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 66 - DNA, 11862 bp, Artificial Sequence
  Pol-IRES (EMCV)-Core Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 67 - DNA, 9490 bp, Artificial Sequence
  preS1-2A-PreS2.S Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggaagcggagctactaacttcagcctgctgaagcaggctggagacgtggaggagaaccctggacctatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 68 - DNA, 10174 bp, Artificial Sequence
  PreS2.S-IRES (EV71)-preS1 Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 69 - DNA, 12730 bp, Artificial Sequence
  PreS2.S-2A-preS1-2A-Core-2A-Pol Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattggaagcggagctactaacttcagcctgctgaagcaggctggagacgtggaggagaaccctggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggatcaggcgctacgaattttagccttctgaagcaagcgggagacgttgaagaaaacccagggcctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgtgggtccggagcgactaacttttccctgctgaaacaagcgggtgacgtcgaagagaatccgggacctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccataaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 70 - DNA, 12730 bp, Artificial Sequence
  PreS2.S-2A-preS1-2A-Pol-2A-Core Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaggatcaggcgctacgaattttagccttctgaagcaagcgggagacgttgaagaaaacccagggcctatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 71 - DNA, 13254 bp, Artificial Sequence
  Pol-2A-Core-IRES (EMCV)-PreS2.S-2A-preS1 Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 72 - DNA, 13414 bp, Artificial Sequence
  Pol-2A-Core-IRES (EV71)-PreS2.S-2A-preS1 Replicon
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatggctcgacctctgtgtaccctgctactcctgatggctaccctggctggagctctggccagcatgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcaaacgccacggggatgagtccttttgcagccagtctagtggcatcctgagcagatcccccgtggggccttgtattcagtctcagctgcggaagagtagactgggactgcagccacagcagggacacctggcacgacggcagcagggaaggtctggcagtatccgggctagagtgcatcccacaactagaaggaccttcggcgtcgagccatcaggaagcggccacatcgacaacagcgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctccaggaagcggagctactaacttcagcctgctgaagcaggctggagacgtggaggagaaccctggacctatggctcgacctctgtgtaccctgctactcctgatggctaccctggctggagctctggccagcgacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaattccagcacaagggactagtatgttcccctcctgctgttgtactaagccaagcgacggtaattgcacgtgtatcccaatcccgtcctcctgggcgtttgccaagtacctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatattgggtccggagcgactaacttttccctgctgaaacaagcgggtgacgtcgaagagaatccgggacctatggctaggcccctgtgtacacttttgctcctgatggccaccctcgctggagctctggcaagcggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggaccactggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcataaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 73 - DNA, 18 bp, Artificial Sequence
  T7 promoter
taatacgactcactatag

SEQ ID NO: 74 - protein, 4 aa, Artificial Sequence
  YXDD motif
  misc_feature (2)..(2): Xaa can be any naturally occurring amino acid
YXDD

SEQ ID NO: 75 - protein, 4 aa, Artificial Sequence
  DEDD motif
DEDD

SEQ ID NO: 76 - protein, 32 aa, Artificial Sequence
  Core antigen C terminal deletion
RGRSPRRRTPSPRRRRSQSPRRRRSQSRESQC

SEQ ID NO: 77 - protein, 21 aa, Artificial Sequence
  Cystatin S signal peptide
MARPLCTLLLLMATLAGALAS

SEQ ID NO: 78 - DNA, 681 bp, Artificial Sequence
  S-surface antigen coding sequence
atggagaatatcacctccgggtttctgggcccactcctggtccttcaggccggattcttcctgctgactcgaatcctcaccataccccagagcctggacagctggtggacaagcctgaattttctgggaggaactcctgtatgcctgggacaaaattcacagtcccctacaagtaaccattcaccgacaagttgtcctcccatctgtcccggatacaggtggatgtgcctgcgaaggttcatcatcttcctcttcatcctcttgctttgccttattttcctcctggttcttctggactatcagggcatgctgcctgtgtgcccactgataccaggatctagtactaccagcacaggcccgtgtaagacctgtacaactccagcacaagggactagtatgttcccctcctgctgttgtactaagccatcagacggtaattgcacgtgtatcccaatcccgtcctcctgggcgtttgccaagtttctctgggaatgggcctcagtcagattttcatggcttagtcttttggtgccgttcgtgcagtggtttgtgggactctctccgactgtgtggctcagcgtgatctggatgatgtggtactggggcccttccctttacaacatactgtctccattccttcccctgctgccaatcttcttttgcctgtgggtctatatttaa

SEQ ID NO: 79 - protein, 226 aa, Artificial Sequence
  S-surface antigen
MENITSGFLGPLLVLQAGFFLLTRILTIPQSLDSWWTSLNFLGGTPVCLGQNSQSPTSNHSPTSCPPICPGYRWMCLRRFIIFLFILLLCLIFLLVLLDYQGMLPVCPLIPGSSTTSTGPCKTCTTPAQGTSMFPSCCCTKPSDGNCTCIPIPSSWAFAKFLWEWASVRFSWLSLLVPFVQWFVGLSPTVWLSVIWMMWYWGPSLYNILSPFLPLLPIFFCLWVYI

SEQ ID NO: 80 - DNA, 567 bp, Artificial Sequence
  LM surface antigen coding sequence
ggtggatggagctcaaagccgcggaaagggatgggtactaacctgtccgtaccaaatcccctgggattttttccagaccaccaactcgatcctgcttttggcgcaaattccaacaatcccgactgggactttaaccctaacaaggacacctggcctgatgccaacaaggtgggggcaggagcctttggtcccggcttcaccccaccccatggaggtcttttgggatggtcaccacaggcccagggcatcctgaccactgtccctgctgctccaccgccagcttctactaatcgacagagcgggaggcagccgacccccctgagtccccccctgcgggatacccaccctcaggcaatgcagtggaactcaactactttccatcagacccttcaggaccctagagtgcgcgggctgtactttcctgctgggggaagcagtagcgggaccgttaatccagtacctacgaccgcctctcccatatcttctatctttagtaggactggtgaccctgctcccaacatggaaaatataactagcggcttcctgggcccccttctcgtcctgtaa

SEQ ID NO: 81 - protein, 188 aa, Artificial Sequence
  LM surface antigen
GGWSSKPRKGMGTNLSVPNPLGFFPDHQLDPAFGANSNNPDWDFNPNKDTWPDANKVGAGAFGPGFTPPHGGLLGWSPQAQGILTTVPAAPPPASTNRQSGRQPTPLSPPLRDTHPQAMQWNSTTFHQTLQDPRVRGLYFPAGGSSSGTVNPVPTTASPISSIFSRTGDPAPNMENITSGFLGPLLVL

SEQ ID NO: 82 - protein, 280 aa, Artificial Sequence
  M surface antigen
QWNSTTFHQTLQDPRVRGLYFPAGGSSSGTVNPVPTTASPISSIFSRTGDPAPNMENITSGFLGPLLVLQAGFFLLTRILTIPQSLDSWWTSLNFLGGTPVCLGQNSQSPTSNHSPTSCPPICPGYRWMCLRRFIIFLFILLLCLIFLLVLLDYQGMLPVCPLIPGSSTTSTGPCKTCTTPAQGTSMFPSCCCTKPSDGNCTCIPIPSSWAFAKFLWEWASVRFSWLSLLVPFVQWFVGLSPTVWLSVIWMMWYWGPSLYNILSPFLPLLPIFFCLWVYI

SEQ ID NO: 83 - protein, 399 aa, Artificial Sequence
  L surface antigen
GGWSSKPRKGMGTNLSVPNPLGFFPDHQLDPAFGANSNNPDWDFNPNKDTWPDANKVGAGAFGPGFTPPHGGLLGWSPQAQGILTTVPAAPPPASTNRQSGRQPTPLSPPLRDTHPQAMQWNSTTFHQTLQDPRVRGLYFPAGGSSSGTVNPVPTTASPISSIFSRTGDPAPNMENITSGFLGPLLVLQAGFFLLTRILTIPQSLDSWWTSLNFLGGTPVCLGQNSQSPTSNHSPTSCPPICPGYRWMCLRRFIIFLFILLLCLIFLLVLLDYQGMLPVCPLIPGSSTTSTGPCKTCTTPAQGTSMFPSCCCTKPSDGNCTCIPIPSSWAFAKFLWEWASVRFSWLSLLVPFVQWFVGLSPTVWLSVIWMMWYWGPSLYNILSPFLPLLPIFFCLWVYI

SEQ ID NO: 84 - protein, 149 aa, Artificial Sequence
  Core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVR

SEQ ID NO: 85 - protein, 150 aa, Artificial Sequence
  Core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRR

SEQ ID NO: 86 - protein, 151 aa, Artificial Sequence
  Core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRR

SEQ ID NO: 87 - DNA, 447 bp, Artificial Sequence
  Core antigen coding sequence
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgt

SEQ ID NO: 88 - DNA, 450 bp, Artificial Sequence
  Core antigen coding sequence
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgc

SEQ ID NO: 89 - DNA, 456 bp, Artificial Sequence
  Core antigen coding sequence
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtccgtcgccgttaa

SEQ ID NO: 90 - DNA, 63 bp, Artificial Sequence
  Cystatin S signal peptide coding sequence
atggctcgacctctgtgtaccctgctactcctgatggctaccctggctggagctctggccagc

SEQ ID NO: 91 - protein, 4 aa, Artificial Sequence
  HBV Core antigen C-terminal
VVRR

SEQ ID NO: 92 - protein, 5 aa, Artificial Sequence
  HBV Core antigen C-terminal
VVRRR

(19) United States
(12) Patent Application Publication
(10) Pub. No.: US 2022/0016237 A1
(43) Pub. Date: Jan. 20, 2022

(54) RNA REPLICON VACCINES AGAINST HBV

(71) Applicant: Janssen Sciences Ireland Unlimited Company, Co Cork (IE)

(72) Inventors: Jason L. DEHART, San Diego, CA (US); Nathaniel Stephen WANG, San Diego, CA (US); Parinaz ALIAHMAD, San Diego, CA (US); Christian MAINE, San Diego, CA (US); Heather Lynn DAVIS, Beerse (BE); Craig PACE, South San Francisco, CA (US)

(21) Appl. No.: 17/370,337

(22) Filed: Jul. 8, 2021

Related U.S. Application Data
(60) Provisional application No. 63/049,400, filed on Jul. 8, 2020.
(60) Provisional application No. 63/144,051, filed on Feb. 1, 2021.

(51) Int. Cl. A61K 39/29; C12N 7/00; A61P 31/20

(52) U.S. Cl. CPC A61K 39/292; A61K 2039/53; A61P 31/20; C12N 7/00

(57) ABSTRACT

Nucleic acid molecules encoding hepatitis B virus (HBV) surface antigens, HBV core antigens, and HBV polymerase antigens, and related combinations, are described. Also described are vectors, such as DNA plasmids or viral vectors, and RNA replicons, expressing the HBV antigens, and pharmaceutical compositions containing the expression vectors. Methods of inducing an immune response against HBV or treating an HBV-induced disease, particularly in individuals having chronic HBV infection, using the pharmaceutical compositions of the invention are also described.

### CROSS-REFERENCE TO RELATED APPLICATIONS

This application claims priority to U.S. Provisional Patent Application No. 63/049,400, filed Jul. 8, 2020, and U.S. Provisional Patent Application No. 63/144,051, filed Feb. 1, 2021, the disclosures of which are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “TIP1088USNP1-Sequence_Listing” and a creation date of Jul. 2, 2021, and having a size of 389 KB. The sequence listing submitted via EFS-Web is part of the specification and is herein incorporated by reference in its entirety.

### FIELD OF THE INVENTION

The present disclosure relates generally to the field of molecular biology and genetic engineering, including nucleic acid molecules useful for regulating gene expression, and the use of the nucleic acid molecules for, for example, production of desired products in suitable host cells in cell culture or in a subject, and for conferring beneficial characteristics to the host cells or subjects.

### BACKGROUND OF THE INVENTION

Hepatitis B virus (HBV) is a small 3.2-kb hepatotropic DNA virus that encodes four open reading frames and seven proteins. About two billion people are infected with HBV, and approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204; 2015 March). The World Health Organization recommends vaccination of all infants, and, in countries where there is low or intermediate hepatitis B endemicity, vaccination of all children and adolescents (<18 years of age), and of people of certain at-risk population categories. Due to vaccination, worldwide infection rates have dropped dramatically. However, prophylactic vaccines do not cure established HBV infection.

Chronic HBV is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure may be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-α has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing immunogenic compositions and methods for inducing an immune response against hepatitis B viruses (HBV) infection. The immunogenic compositions and methods of the invention can be used to provide therapeutic immunity to a subject, such as a subject having chronic HBV infection.

In a general aspect, the application relates to a nucleic acid molecule or combination comprising a non-naturally occurring polynucleotide sequence. In some embodiments, the non-naturally occurring polynucleotide sequence comprises, ordered from the 5′- to 3′-end:
(1) a polynucleotide sequence encoding a first hepatitis B virus (HBV) antigen,
(2) a first internal ribosome entry sequence (IRES) element or a polynucleotide sequence encoding a first autoprotease peptide, and
a polynucleotide sequence encoding a second HBV antigen.

wherein the first HBV antigen and the second HBV antigen are each independently selected from the group consisting of an HBV core antigen, an HBV polymerase (pol) antigen, and an HBV surface antigen, and at least one of the first and second HBV antigens is an HBV surface antigen, preferably an HBV Pre-S1 antigen or an HBV PreS2.S antigen.

In an embodiment, one of the first or second HBV antigens is an HBV core or an HBV pol antigen.

In an embodiment, the non-naturally occurring polynucleotide sequence further comprises, ordered from the 5′- to 3′-end:
(4) a second IRES element or a polynucleotide sequence encoding a second autoprotease peptide operably linked to the 3′ end of the polynucleotide sequence encoding the second HBV antigen, and
(5) a polynucleotide sequence encoding a third HBV antigen independently selected from the group consisting of an HBV core antigen, an HBV pol antigen, and an HBV surface antigen.

In another embodiment, the non-naturally occurring polynucleotide sequence further comprises, ordered from the 5′- to 3′-end:
(6) a third IRES element or a polynucleotide sequence encoding a third autoprotease peptide operably linked to the 3′ end of the polynucleotide sequence encoding the third HBV antigen, and
(7) a polynucleotide sequence encoding a fourth HBV antigen independently selected from the group consisting of an HBV core antigen, an HBV pol antigen, and an HBV surface antigen.

In another embodiment, the nucleic acid molecule or combination comprises a first non-naturally occurring polynucleotide sequence comprising, ordered from the 5′- to 3′-end:
(1) a polynucleotide sequence encoding a first hepatitis B virus (HBV) antigen,
(2) a first internal ribosome entry sequence (IRES) element or a polynucleotide sequence encoding a first autoprotease peptide, and
(3) a polynucleotide sequence encoding a second HBV antigen, and
a second non-naturally occurring polynucleotide sequence comprising, ordered from the 5′- to 3′-end:
(1) a polynucleotide sequence encoding a third hepatitis B virus (HBV) antigen,
(2) a second internal ribosome entry sequence (IRES) element or a polynucleotide sequence encoding a second autoprotease peptide, and
(3) a polynucleotide sequence encoding a fourth HBV antigen,

wherein the first and second non-naturally occurring polynucleotide sequence are linked by a third internal ribosome entry sequence (IRES) element or a polynucleotide sequence encoding a third autoprotease peptide, or are present in separate nucleic acid molecules, and

wherein the first, second, third and fourth HBV antigens are each independently selected from the group consisting of an HBV core antigen, an HBV polymerase (pol) antigen, and an HBV surface antigen, and at least one of the first, second, third and fourth HBV antigens is an HBV surface antigen selected from an HBV Pre-S1 antigen having an amino acid sequence at least 98% identical to the amino acid sequence of SEQ ID NO: 1 or SEQ ID NO: 3 and an HBV PreS2.S antigen having an amino acid sequence at least 98% identical to the amino acid sequence of SEQ ID NO: 5, preferably one of the first, second, third or fourth HBV antigens is an HBV core or an HBV pol antigen.

In another embodiment, each of the first, second, third and fourth HBV antigens is different from each other.

In another embodiment, each of the first, second, third and fourth HBV antigens is independently selected from the group consisting of:
(i) a first HBV Pre-S1 antigen comprising, preferably consisting of, an amino acid sequence that is at least 98% identical to the amino acid sequence of SEQ ID NO: 1, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 1;
(ii) a second HBV Pre-S1 antigen comprising, preferably consisting of, an amino acid sequence that is at least 98% identical to the amino acid sequence of SEQ ID NO: 3, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 3;
(iii) an HBV PreS2.S antigen comprising, preferably consisting of, an amino acid sequence that is at least 98% identical to the amino acid sequence of SEQ ID NO: 5, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 5;
(iv) an HBV core antigen comprising, preferably consisting of, an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7; and
(v) an HBV polymerase antigen comprising, preferably consisting of, an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9,
preferably, each of the first and second HBV Pre-S1 antigens, the HBV core antigen and the HBV pol antigen is independently operably linked to a signal peptide, and the HBV PreS2.S antigen comprises an internal signal peptide.

In some embodiments, the HBV core antigen comprises, preferably consists of, an amino acid sequence that is at least 98% identical to at least one of SEQ ID NOs: 84, 85, or 86, such as at least 98%, at least 99%, or 100% identical to SEQ ID NOs: 84, 85, or 86. In some embodiments, the last five C-terminal amino acids of the HBV core antigen comprise a VVR amino acid sequence, more particularly a VVRR (SEQ ID NO: 91) amino acid sequence, more particularly a VVRRR (SEQ ID NO: 92) amino acid sequence.

In some embodiments, each of the HBV surface antigen, the HBV core antigen and the HBV pol antigen comprises:
(i) a consensus sequence for HBV genotypes A, B, C and D; and/or
(ii) one or more epitopes for HLA-A*11:01, HLA-A*24:02, HLA-A*02:01, HLA-A*A2402, HLA-A*A0101, or HLA-B*40:01.

In some embodiments, each of the HBV surface antigens, the HBV core antigen and the HBV pol antigen comprises one or more epitopes for HLA-A*11:01.

In another embodiment, each of the first, second, third and fourth HBV antigens is independently selected from the group consisting of:
(i) the first HBV Pre-S1 antigen consisting of the amino acid sequence of SEQ ID NO: 1;
(ii) the second HBV Pre-S1 antigen consisting of the amino acid sequence of SEQ ID NO: 3;
(iii) the HBV PreS2.S antigen consisting of the amino acid sequence of SEQ ID NO: 5;
(iv) the HBV core antigen consists of the amino acid sequence of SEQ ID NO: 84, SEQ ID NO: 85, or SEQ ID NO: 86; and
(v) the HBV pol antigen consisting of the amino acid sequence of SEQ ID NO: 9, preferably, each of the first and second HBV Pre-S1 antigens, the HBV core antigen and the HBV pol antigen is independently operably linked to a signal peptide, such as the signal peptide comprising the amino acid sequence of SEQ ID NO: 77.

In another embodiment, each of the polynucleotide sequences encoding the first, second, third and fourth HBV antigens is independently selected from the group consisting of:
(i) a polynucleotide sequence encoding the first HBV Pre-S1 antigen having a sequence that is at least 90% identical to SEQ ID NO: 2, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2;
(ii) a polynucleotide sequence encoding the second HBV Pre-S1 antigen having a sequence that is at least 90% identical to SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4;
(iii) a polynucleotide sequence encoding the HBV PreS2.S antigen having a sequence that is at least 90% identical to SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 6;
(iv) a polynucleotide sequence encoding the HBV core antigen having a sequence that is at least 90% identical to SEQ ID NO: 8, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 8; and
(v) the polynucleotide sequence encoding the HBV pol antigen having a sequence that is at least 90% identical to SEQ ID NO: 10, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 10,
preferably, the polynucleotide sequence encoding each of the first and second HBV Pre-S1 antigens, the HBV core antigen and the HBV pol antigen is independently operably linked to a polynucleotide sequence encoding a signal peptide, and the HBV PreS2.S antigen comprises an internal signal peptide.

In some embodiments, each of the polynucleotide sequences encoding the first, second, third and fourth HBV antigens is independently selected from the group consisting of:
(i) a polynucleotide sequence encoding the first HBV Pre-S1 antigen consisting of the sequence of SEQ ID NO: 2;
(ii) a polynucleotide sequence encoding the second HBV Pre-S1 antigen consisting of the sequence of SEQ ID NO: 4;
(iii) a polynucleotide sequence encoding the HBV PreS2.S antigen consisting of the sequence of SEQ ID NO: 6;
(iv) a polynucleotide sequence encoding the HBV core antigen consisting of the sequence of any one of SEQ ID NO: 87, SEQ ID NO: 88 or SEQ ID NO: 89; and
(v) a polynucleotide sequence encoding the HBV pol antigen consisting of the sequence of SEQ ID NO: 10;
preferably, the polynucleotide sequence encoding each of the first and second HBV Pre-S1 antigens, the HBV core antigen and the HBV pol antigen is independently operably linked to a polynucleotide encoding a signal peptide, such as the polynucleotide comprising the sequence of SEQ ID NO: 90.

In an embodiment, each of the first, second and third autoprotease peptides independently comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof. Preferably, each of the first, second and third autoprotease peptides comprises the peptide sequence of P2A, such as a P2A sequence of SEQ ID NO: 11.

In another embodiment, each of the first, second and third IRES is derived from encephalomyocarditis virus (EMCV) or Enterovirus 71 (EV71). Preferably, each of the first, second and third IRES comprises the polynucleotide sequence of SEQ ID NO: 13 or 14.

In some embodiments, the nucleic acid molecule or combination comprises a non-naturally occurring polynucleotide sequence comprising, ordered from the 5′- to 3′-end:
(1) a polynucleotide sequence encoding an HBV Pre-S1 antigen having the amino acid sequence of SEQ ID NO: 1 or 3, a polynucleotide sequence encoding a P2A amino acid sequence of SEQ ID NO: 11 or an IRES having the polynucleotide sequence of SEQ ID NO: 13 or 14, and a polynucleotide sequence encoding an HBV PreS2.S antigen having the amino acid sequence of SEQ ID NO: 5;
(2) a polynucleotide sequence encoding an HBV Pre S2.S antigen having the amino acid sequence of SEQ ID NO: 5, a polynucleotide sequence encoding a P2A amino acid sequence of SEQ ID NO: 11 or an IRES having the polynucleotide sequence of SEQ ID NO: 13 or 14, and a polynucleotide sequence encoding an HBV Pre-S1 antigen having the amino acid sequence of SEQ ID NO: 1 or 3;
(3) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(4) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(5) a polynucleotide sequence encoding an HBV Pre S2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(6) a polynucleotide sequence encoding an HBV Pre S2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(7) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(8) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(9) a polynucleotide sequence encoding an HBV Pre S2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(10) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(11) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(12) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(13) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(14) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(15) a polynucleotide sequence encoding an HBV PreS2.S antigen having the amino acid sequence of SEQ ID NO: 5, an IRES having the polynucleotide sequence of SEQ ID NO: 13 or 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(16) a polynucleotide sequence encoding an HBV PreS2.S antigen having the amino acid sequence of SEQ ID NO: 5, an IRES having the polynucleotide sequence of SEQ ID NO: 14, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(17) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 13 or 14, and a polynucleotide sequence encoding an HBV Pre S2.S antigen having the amino acid sequence of SEQ ID NO: 5;
(18) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13 or 14, and a polynucleotide sequence encoding an HBV Pre S2.S antigen having the amino acid sequence of SEQ ID NO: 5;
(19) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(20) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(21) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV PreS2.S antigen having the amino acid sequence of SEQ ID NO: 5; or
(22) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV PreS2.S antigen having the amino acid sequence of SEQ ID NO: 5,
preferably, the polynucleotide sequence encoding each of the first and second HBV Pre-S1 antigens, the HBV core antigen and the HBV pol antigen is independently operably linked to a polynucleotide sequence encoding a signal peptide, such as the signal peptide comprising the amino acid sequence of SEQ ID NO: 77.

In an embodiment, the nucleic acid molecule or combination comprises the non-naturally occurring polynucleotide sequence of any one of SEQ ID NOs: 15 to 54.

In another general aspect, the application relates to a vector comprising a nucleic acid molecule or combination of the application.

In an embodiment, the vector is a DNA plasmid. In another embodiment, the vector is a DNA viral vector or an RNA viral vector. In an embodiment, the vector is a Modified Vaccinia Ankara (MVA) vector or an adenovirus vector. In an embodiment, the vector is an Ad26, Ad35, or MVA-BN vector.

In another general aspect, the application relates to an RNA replicon, comprising, ordered from the 5′- to 3′-end:
(1) a 5′ untranslated region (5′-UTR) required for nonstructural protein-mediated amplification of an RNA virus;
(2) a polynucleotide sequence encoding at least one, preferably all, of non-structural proteins of the RNA virus;
(3) a subgenomic promoter of the RNA virus;
(4) a nucleic acid molecule or combination of the application; and
(5) a 3′ untranslated region (3′-UTR) required for nonstructural protein-mediated amplification of the RNA virus.

In another general aspect, the application relates to an RNA replicon, comprising, ordered from the 5′- to 3′-end,
(1) an alphavirus 5′ untranslated region (5′-UTR),
(2) a 5′ replication sequence of an alphavirus non-structural gene nsp1,
(3) a downstream loop (DLP) motif of a virus species,
(4) a polynucleotide sequence encoding a fourth autoprotease peptide,
(5) a polynucleotide sequence encoding alphavirus non-structural proteins nsp1, nsp2, nsp3 and nsp4,
(6) an alphavirus subgenomic promoter,
(7) a nucleic acid molecule or combination of the application,
(8) an alphavirus 3′ untranslated region (3′ UTR), and
(9) optionally, a poly adenosine sequence.

In an embodiment, the DLP motif is from a virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MTDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (U AV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus.

In another embodiment, the fourth autoprotease peptide is selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof. Preferably, the fourth autoprotease peptide comprises the peptide sequence of P2A.

In another general aspect, the application relates to an RNA replicon, comprising, ordered from the 5′- to 3′-end,
(1) a 5′-UTR having the polynucleotide sequence of SEQ ID NO: 55,
(2) a 5′ replication sequence having the polynucleotide sequence of SEQ ID NO: 56,
(3) a DLP motif comprising the polynucleotide sequence of SEQ ID NO: 57,
(4) a polynucleotide sequence encoding a P2A sequence of SEQ ID NO: 11,
(5) polynucleotide sequences encoding alphavirus non-structural proteins nsp1, nsp2, nsp3 and nsp4 having the nucleic acid sequences of SEQ ID NO: 58, SEQ ID NO: 59, SEQ ID NO: 60 and SEQ ID NO: 61, respectively,
(6) a subgenomic promoter having polynucleotide sequence of SEQ ID NO: 62,
(7) a nucleic acid molecule or combination of the application, and
(8) a 3′ UTR having the polynucleotide sequence of SEQ ID NO: 63.

In an embodiment, the polynucleotide sequence encoding the P2A sequence comprises SEQ ID NO: 12, the nucleic acid molecule or combination comprises the polynucleotide sequence of any one of SEQ ID NOs: 15 to 54, and the RNA replicon further comprises a poly adenosine sequence at the 3′-end of the replicon. Preferably, the poly adenosine sequence has the sequence of SEQ ID NO: 64.

In another general aspect, the application relates to an RNA replicon comprising the polynucleotide sequence of any one of SEQ ID NOs: 65 to 72.

In another general aspect, the application relates to a nucleic acid molecule comprising a polynucleotide sequence encoding an RNA replicon of the application. Preferably, the nucleic acid further comprises a T7 promoter operably linked to the 5′-end of the DNA sequence. More preferably, the T7 promoter comprises the nucleotide sequence of SEQ ID NO: 73.

In another general aspect, the application relates to a pharmaceutical composition comprising a nucleic acid molecule or combination, a vector, or an RNA replicon of the application, and a pharmaceutically acceptable carrier.

In an embodiment, the pharmaceutically acceptable carrier comprises a lipid nanoparticle, preferably the lipid nanoparticle comprises one or more of ALC-0315, DOTMA, DOTAP, DDAB, DOGS, DSDMA, DODMA, DLinDMA, DLenDMA, γ-DLenDMA, DLin-K-DMA, DLin-K-C2-DMA, DLin-K-C3-DMA, DLin-K-C4-DMA, DLen-C2K-DMA, y-DLen-C2K-DMA, DLin-M-C2-DMA, DLin-M-C3-DMA, DLin-MP-DMA, or DCChol.

In another embodiment, the pharmaceutical composition further comprises: (1) a polynucleotide sequence encoding an HBV core antigen having the amino acid sequence of any one of SEQ ID NOs: 84, 85 or 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen having, preferably consisting of, the amino acid sequence of SEQ ID NO: 9; or (2) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen having the amino acid sequence of any one of SEQ ID NOs: 84, 85 or 86.

In another general aspect, the application relates to a method for vaccinating a subject against HBV, the method comprising administering to the subject a pharmaceutical composition of the application. In an embodiment, the method further comprises administering to the subject a second composition comprising a nucleic acid molecule or combination encoding at least one identical HBV antigen as a prime-boost regimen. In some embodiments, the prime-boost regimen comprises a priming composition comprising an RNA replicon of the application and a boosting composition comprising a vector which is not an RNA replicon, and which encodes at least one identical HBV epitope, preferably, at least one identical HBV antigen, as the priming composition. In an embodiment, the boosting composition comprises a Modified Vaccinia Ankara (MVA) vector, an adenovirus vector or a plasmid vector. In some embodiments, the boosting composition comprises an Ad26, Ad35, or MVA-BN vector. In some embodiments, the prime-boost regimen comprises a boosting composition comprising an RNA replicon of the application and a priming composition comprising a vector which is not an RNA replicon, and which encodes at least one identical HBV epitope, such as at least one identical HLA epitope, preferably, at least one identical HBV antigen, as the boosting composition. In an embodiment, the priming composition comprises a Modified Vaccinia Ankara (MVA) vector, an adenovirus vector or a plasmid vector. In some embodiments, the priming composition comprises an Ad26, Ad35, or MVA-BN vector.

In another general aspect, the application relates to a method for reducing infection and/or replication of HBV in a subject, comprising administering to the subject a pharmaceutical composition of the application or vaccinating the subject according to methods of the application.

In another general aspect, the application relates to an isolated host cell comprising a nucleic acid molecule or combination, a vector, or an RNA replicon of the application.

In another general aspect, the application relates to a method of producing an RNA replicon, comprising transcribing a nucleic acid of the application, in vivo or in vitro.

In another general aspect, the application relates to a pharmaceutical composition of the application for use in inducing an immune response against a hepatitis B virus (HBV) in a subject in need thereof, preferably the subject has chronic HBV infection, optionally in combination with another immunogenic agent, preferably another anti-HBV agent.

In another general aspect, the application relates to a pharmaceutical composition of the application for use in treating a hepatitis B virus (HBV)-induced disease in a subject in need thereof, preferably the subject has chronic HBV infection, and the HBV-induced disease is selected from the group consisting of advanced fibrosis, cirrhosis and hepatocellular carcinoma (HCC), optionally in combination with another therapeutic agent, preferably another anti-HBV agent.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1A shows a schematic of the antigen designs, including Pol with inactivating point mutations, truncated Core with C terminal deletion (SEQ ID NO: 76), PreS2.S, and PreS1 showing the cystatin S signal peptide (SEQ ID NO: 77)

FIG. 1B shows a schematic of bicistronic and tetracistronic vaccine designs.

FIG. 2 is a series of graphs showing relative expression of HBV antigens from the top 4 bicistronic and top 4 tetracistronic vaccine designs. Core, PreS2.S and PreS1 expression in Vero cells was measured by flow cytometry and reported as MFI relative to monogenic controls. Pol expression was measured by Western blot, and relative expression determined using densitometry.

FIG. 3 is a series of graphs showing relative expression of HBV antigens from tricistronic vaccine designs. Core and PreS2.S expression in Vero cells was measured by flow cytometry and reported as MFI relative to monogenic controls. Pol expression was measured by Western blot, and relative expression determined using densitometry.

FIG. 4A-FIG. 4D are graphs showing the results of in vivo immunization with SMARRT replicon-encoding HBV antigens. C57BL/6 mice were injected i.m. with SMARRT replicon encoding monogenic HBV antigens or admixed. Saline was injected as a control group. 14 days post-prime the spleens were harvested and splenocytes were restimulated with overlapping peptide pools for Core (FIG. 4A), Pol (FIG. 4B), PreS2.S (FIG. 4C) and PreS1 (FIG. 4D). The number of IFNγ producing cells was measured by ELISpot. Graphs show mean with 95% CI (n=5 mice per group), Mann-Whitney test was performed for statistical comparisons *p<0.05; **p<0.01

FIG. 5A-FIG. 5H are graphs showing the results of in vivo immunization and Week 2 restimulation with SMARRT replicon-encoding HBV antigens. C57BL/6 mice were injected i.m. with SMARRT replicon-encoding monogenic HBV antigens or admixed. Saline was injected as a control group. 14 days post-prime, the spleens were harvested and splenocytes were restimulated with overlapping peptide pools for core (FIG. 5A & FIG. 5E), Pol (FIG. 5B & FIG. 5F), PreS2.S (FIG. 5C & FIG. 5G) and PreS1 (FIG. 5D & FIG. 5H) in the presence of brefeldin A for 6 hours. IFNγ, TNFα and IL-2 production by CD4 and CD8 T cells was measured by intracellular cytokine staining. Polyfunctionality is plotted as determined by the production of 1 (IFNγ+), 2 (IFNγ+TNFα+) or 3 (IFNγ+TNFα+IL-2+) cytokines per cell. Graphs show mean with SD (n=5 mice per group).

FIG. 6A-FIG. 6D are graphs showing the results of in vivo immunization with SMARRT replicon-encoding HBV antigens. C57BL/6 mice were injected i.m. with SMARRT replicon encoding monogenic or admixed HBV antigens, bigenic antigens, or tetracistronic constructs. Saline was injected as a control group. 14 days post-prime the spleens were harvested and splenocytes were restimulated with overlapping peptide pools for core (FIG. 6A), Pol (FIG. 6B), PreS2.S (FIG. 6C) and PreS1 (FIG. 6D). The number of IFNγ producing cells was measured by ELISpot. Graphs show mean with 95% CI (n=5 mice per group).

FIG. 7A-FIG. 7D are graphs showing the results of in vivo immunization and Week 2 restimulation with SMARRT replicon-encoding HBV antigens. C57BL/6 mice were injected i.m. with SMARRT replicon-encoding monogenic or admixed HBV antigens, bigenic antigens, or tetracistronic constructs. Saline was injected as a control group. 14 days post-prime, the spleens were harvested and splenocytes were restimulated with overlapping peptide pools for Core (FIG. 7A), Pol (FIG. 7B), PreS2.S (FIG. 7C) and PreS1 (FIG. 7D) in the presence of brefeldin A for 6 hours. IFNγ, TNFα and IL-2 production by CD4 and CD8 T cells was measured by intracellular cytokine staining. Polyfunctionality is plotted as determined by the production of 1 (IFNγ+), 2 (IFNγ+TNFα+) or 3 (IFNγ+TNFα+IL-2+) cytokines per cell. Graphs show mean with SD (n=5 mice per group).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value, such as a % sequence identity or a % sequence identity range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

An “epitope” as used herein is a set of amino acid residues that form a site recognized by an immunoglobulin, T cell receptor or human leukocyte antigen (HLA) molecule.

The HLA proteins are encoded by clusters of genes that form a region located on chromosome 6 known as the Major Histocompatibility Complex (MHC), in recognition of the important role of the proteins encoded by the MHC loci in graft rejection. Accordingly, the HLA proteins are also referred to as MHC proteins. HLA or MHC proteins are cell surface glycoproteins that bind peptides at intracellular locations and deliver them to the cell surface, where the combined ligand is recognized by a T cell. Class I MHC proteins are found on virtually all of the nucleated cells of the body. The class I MHC proteins bind peptides present in the cytosol and form peptide-MHC protein complexes that are presented at the cell surface, where they are recognized by cytotoxic CD8+ T cells. Class II MHC proteins are usually found only on antigen-presenting cells such as B lymphocytes, macrophages, and dendritic cells. Each MHC Class I receptor consists of a variable a chain and a relatively conserved 132-microglobulin chain. Three different, highly polymorphic class I a chain genes have been identified. These are called HLA-A, HLA-B, and HLA-C. Variations in the a chain accounts for all of the different class I MHC genes in the population.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The same determination may be made for nucleotide sequences. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration of two or more therapies or components, such as two nucleic acid molecules, e.g., RNA replicon, or an immunogenic composition and an adjuvant. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. The use of the term “in combination with” does not restrict the order in which therapies or components are administered to a subject. For example, a first therapy or component (e.g. first nucleic acid molecule) can be administered prior to (e.g., 5 minutes to one hour before), concomitantly with or simultaneously with, or subsequent to (e.g., 5 minutes to one hour after) the administration of a second therapy or component (e.g., second nucleic acid molecule). In some embodiments, a first therapy or component (e.g. first nucleic acid molecule) and a second therapy or component (e.g., e.g., second nucleic acid molecule) are administered in the same composition. In other embodiments, a first therapy or component (e.g. first nucleic acid molecule) and a second therapy or component (e.g., e.g., second nucleic acid molecule) are administered in separate compositions.

As used herein, a “non-naturally occurring” nucleic acid or polypeptide refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, the term “operably linked” refer to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

As used herein, the term “priming composition”, “priming immunization” or “prime immunization” refers to primary antigen stimulation by using a first composition of the invention. Specifically, the term “priming” or “potentiating” an immune response, as used herein, refers to a first immunization using an antigen which induces an immune response to the desired antigen and recalls a higher level of immune response to the desired antigen upon subsequent re-immunization with the same antigen. As used herein, the term “boosting composition”, “boosting immunization” or “boost immunization” refers to an additional immunization administered to, or effective in, a mammal after the primary immunization. Specifically, the term “boosting” an immune response, as used herein, refers to the administration of a composition delivering the same antigen as encoded in the priming immunization.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human. A human subject can include a patient.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of alphavirus replicon RNAs of the application described herein may contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein may equally apply to other components arranged in other orders that can be used in alphavirus replicon RNAs of the application. The application contemplates use of any of the applicable components in any combination having any sequence that can be used in alphavirus replicons of the application, whether or not a particular combination is expressly described.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion form infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the Core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infections virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

### HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response against an HBV in a subject. The induced response can be a humoral and/or cellular mediated response. The HBV antigen can be a polypeptide of HBV, a fragment or epitope thereof, or a combination of multiple HBV polypeptides, portions or derivatives thereof. An HBV antigen is capable of raising in a host a protective immune response, e.g., inducing an immune response against a viral disease or infection, and/or producing an immunity (i.e., vaccinates) a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

### (1) HBV Core Antigen

As used herein, each of the terms “HBV core antigen,” “HBcAg” and “core antigen” refers to an HBV antigen capable of inducing an immune response against an HBV core protein in a subject. The induced immune response can be a humoral and/or cellular mediated response. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four α-helix bundles flanked by an α-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of 31-34 C-terminal amino acid residues of the C-terminal nucleic acid binding domain. In a preferred embodiment, a truncated HBV core antigen comprises a deletion in the C-terminal nucleic acid binding domain, preferably a deletion of 31 C-terminal amino acid residues of the C-terminal nucleic acid binding domain.

In some embodiments, an HBV core antigen amino acid sequence is operably linked to a signal peptide for secretion. Any suitable signal peptide can be used. In one embodiment, an HBV core antigen is operably linked at its N-terminus to a Cystatin S precursor signal peptide, to enhance secretion. In a particular embodiment, the Cystatin S precursor signal peptide has the amino acid sequence of SEQ ID NO: 77. In another particular embodiment, a coding sequence of an HBV core antigen is operably linked to a coding sequence of Cystatin S precursor signal peptide having the polynucleotide sequence of SEQ ID NO: 90.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins as determined by an alignment of amino acid sequences of homologous proteins. The alignment can be conducted using methods or algorithm known in the art, such as using Clustal Omega. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes A, B, C, and D. The term “consensus antigen” is used to refer to an antigen having consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes A, B, C and D. More preferably, a truncated HBV core antigen is capable of inducing a CD8 T cell response in a human subject against at least HBV genotypes A, B, C and D.

In some embodiments, an HBV core antigen of the application comprises one or more T cell epitopes for MHC class I HLA alleles. In some embodiments, an HBV core antigen comprises one or more epitopes selected from the group consisting of HLA-A*11:01 epitopes, HLA-A*02:01 epitopes, HLA-A*A0101 epitopes, and FILA-B*40:01 epitopes. Preferably, an HBV core antigen comprises two or more, such as 2, 3, or 4, of T cell epitopes selected from the group consisting of HLA-A*11:01 epitopes, FHA-A*02:01 epitopes, HLA-A*A0101 epitopes, and HLA-B*40:01 epitopes. More preferably, an HBV core antigen comprises HLA-A*11:01, HLA-A*02:01, FILA-A*A0101, and HLA-B*40:01 T cell epitopes. More preferably, an HBV core antigen comprises one or more HLA-A*11:01 epitope(s).

In some embodiments, an HBV core antigen of the application is a consensus antigen, preferably a consensus antigen derived from at least two, preferably all, of HBV genotypes A, B, C, and D, more preferably a truncated consensus antigen derived from HBV genotypes A, B, C, and D. An exemplary truncated HBV core consensus antigen according to the application consists of an amino acid sequence that is at least 90% identical to SEQ ID NO: 86, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 86. In some embodiments, the HBV core antigen comprises, preferably consists of, an amino acid sequence that is at least 98% identical to SEQ ID NO: 84, SEQ ID NO: 85 or SEQ ID NO: 86, such as at least 98%, 98.5%, 99%, 99.1%, 99.2%, 99.3%, 99.4%, 99.5%, 99.6%, 99.7%, 99.8%, 99.9%, or 100% identical to SEQ ID NO: 84, SEQ ID NO: 85 or SEQ ID NO: 86. SEQ ID NO: 86 is a core consensus antigen derived from HBV genotypes A, B, C, and D. SEQ ID NO: 7 contains a 34-amino acid C-terminal deletion of the highly positively charged (arginine rich) nucleic acid binding domain of the native core antigen. In some preferred embodiments, an HBV core antigen of the application retains one or more of the C-terminal arginine residues and has the amino acid sequence of SEQ ID NO: 84, SEQ ID NO: 85 or SEQ ID NO: 86, to thereby restore the FHA-A*11:01 epitope in the HBV core antigen. In some embodiments, the last five C-terminal amino acids of an HBV core antigen comprise a VVR amino acid sequence, more particularly a VVRR (SEQ ID NO: 91) amino acid sequence, more particularly a VVRRR (SEQ ID NO: 92) amino acid sequence.

In a particular embodiment of the application, an HBV core antigen is a truncated HBV antigen consisting of the amino acid sequence of SEQ ID NO: 7, SEQ ID NO: 84, SEQ ID NO: 85 or SEQ ID NO: 86. In another particular embodiment, an HBV core antigen is encoded by a polynucleotide sequence of SEQ ID NO: 8, SEQ ID NO: 87, SEQ ID NO: 88 or SEQ ID NO: 89, respectively. Preferably, the HBV core antigen consists of the amino acid sequence of SEQ ID NO: 86 and is encoded by the polynucleotide sequence of SEQ ID NO: 89. In some embodiments, an HBV core antigen consists of an amino acid sequence, or is encoded by a polynucleotide sequence, as described in U.S. Patent Application Publication No. US2019/0185828, the content of which is herein incorporated by reference in its entirety.

### (2) HBV Polymerase Antigen

As used herein, the term “HBV polymerase antigen,” “HBV Pol antigen” or “HBV pol antigen” refers to an HBV antigen capable of inducing an immune response against an HBV polymerase in a subject. The immune response can be a humoral and/or cellular mediated response. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and an RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the invention does not have reverse transcriptase activity and RNase H activity, and is capable of inducing an immune response in a mammal against at least two HBV genotypes. Preferably, an HBV Pol antigen is capable of inducing a T cell response in a mammal against at least two, preferably all, of HBV genotypes A, B, C and D. More preferably, a HBV Pol antigen is capable of inducing a CD8 T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif (SEQ ID NO: 74) in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif (SEQ ID NO: 74), the “DEDD” motif (SEQ ID NO: 75) in the RNaseH domain of an HBV pol antigen required for Mg²⁺ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif (SEQ ID NO: 74) of the polymerase domain; and (2) mutating the first aspartate residue (D) to an asparagine residue (N) and the first glutamate residue (E) to a glutamine residue (N) in the “DEDD” motif (SEQ ID NO: 75) of the RNaseH domain, thereby decreasing or substantially eliminating both the reverse transcriptase and RNaseH functions of the pol antigen.

In some embodiments, an HBV pol antigen amino acid sequence is operably linked to a signal peptide for secretion. Any suitable signal peptides can be used. In one embodiment, an HBV pol antigen is operably linked at its N-terminus to a Cystatin S precursor signal peptide, to enhance secretion. In a particular embodiment, the Cystatin S precursor signal peptide has the amino acid sequence of SEQ ID NO: 77. In another particular embodiment, a coding sequence of an HBV pol antigen is operably linked to a coding sequence of Cystatin S precursor signal peptide having the polynucleotide sequence of SEQ ID NO: 90.

In some embodiments, an HBV pol antigen of the application comprises one or more T cell epitopes for MHC class I HLA alleles. In some embodiments, an HBV pol antigen comprises one or more epitopes selected from the group consisting of HLA-A*11:01 epitopes, HLA-A*24:02 epitopes, HLA-A*02:01 epitopes, and HLA-A*A0101 epitopes. Preferably, an HBV pol antigen comprises two or more, such as 2, 3, or 4, T cell epitopes selected from the group consisting of HLA-A*11:01 epitopes, HLA-A*24:02 epitopes, HLA-A*02:01 epitopes, and HLA-A*A0101. More preferably, an HBV pol antigen comprises HLA-A*11:01, HLA-A*24:02, HLA-A*02:01, and HLA-A*A0101 T cell epitopes. More preferably, an HBV pol antigen comprises one or more HLA-A*11:01 epitope(s).

In a preferred embodiment of the application, an HBV pol antigen is a consensus antigen, preferably a consensus antigen derived from at least two, preferably all, of HBV genotypes A, B, C, and D, more preferably an inactivated consensus antigen derived from HBV genotypes A, B, C, and D. An exemplary HBV pol consensus antigen according to the application comprises an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9, preferably at least 98% identical to SEQ ID NO: 9, such as at least 98%, 98.5%, 99%, 99.1%, 99.2%, 99.3%, 99.4%, 99.5%, 99.6%, 99.7%, 99.8%, 99.9% or 100% identical to SEQ ID NO: 9. SEQ ID NO: 9 is a pol consensus antigen derived from HBV genotypes A, B, C, and D comprising four mutations located in the active sites of the polymerase and RNaseH domains. In particular, the four mutations include mutation of the aspartic acid residues (D) to asparagine residues (N) in the “YXDD” motif (SEQ ID NO: 74) of the polymerase domain; and mutation of the first aspartate residue (D) to an asparagine residue (N) and mutation of the glutamate residue (E) to a glutamine residue (Q) in the “DEDD” (SEQ ID NO: 75) motif of the RNaseH domain.

In a particular embodiment of the application, an HBV pol antigen comprises the amino acid sequence of SEQ ID NO: 9. Preferably, an HBV pol antigen consists of the amino acid sequence of SEQ ID NO: 9. In another particular embodiment, an HBV pol antigen is encoded by a polynucleotide sequence of SEQ ID NO: 10. In some embodiments, an HBV pol antigen contains or consists of an amino acid sequence, or is encoded by a polynucleotide sequence as described in U.S. Patent Application Publication No. US2019/0185828, the content of which is herein incorporated by reference in its entirety.

### (3) HBV Surface Antigens

As used herein, each of the terms “HBV surface antigen,” “surface antigen,” “HBV envelope antigen,” “envelope antigen,” and “env antigen” refers to an HBV antigen capable of inducing or eliciting an immune response against one or more HBV surface antigens or envelope proteins in a subject. The immune response can be a humoral and/or cellular mediated response. Each of the terms “HBV surface protein,” “surface protein,” “HBV envelope protein” and “envelope protein” refers to HBV viral surface or envelope proteins. HBV expresses three surface antigens, or envelope proteins. Gene S is the gene of the HBV genome that encodes the surface antigens. The surface antigen gene is one long open reading frame but contains three in frame “start” (ATG) codons that divide the gene into three sections, pre-S1, pre-S2, and S. Because of the multiple start codons, polypeptides of three different sizes called large (L) or L-surface antigen, middle (M) or M-surface antigen, and small (S) or S-surface antigen are produced, also named the HBV L, M and S envelope proteins. Two different promoters (PreS1 and PreS2) drive transcription of the L, M, and 5-surface antigen coding sequences resulting in three different translated proteins, the L, M and S envelope proteins. The PreS2 promoter is sometimes referred to as the PreS2/S promoter since it is driving M-surface antigen and S-surface antigen transcription separately. The amino acid sequence of the L-surface antigen is in-frame with the M and S-surface antigen sequences. Thus, the L-surface antigen contains the M- and S-surface antigen domains and the M-surface antigen includes the S-surface antigen domain. The L-, M- and S-surface antigen are co-C-terminal and share the entire S domain. Relative to S, M has an additional domain, pre-S2, at its N terminus, and relative to M, L has a pre-S1 domain.

In some embodiments, an HBV antigen is an HBV PreS1 antigen, which is encoded by a pre-S1 gene section and contains only the Pre-S1 domain of the L antigen. The PreS1 antigen can have various lengths, such as having 99 to 109 amino acids. An HBV PreS1 antigen of the application can contain the sequence of any naturally occurring PreS1 domain, and variants or derivatives thereof.

In other embodiments, an HBV antigen is an HBV PreS2.S antigen, which is encoded by the pre-S2 and S gene sections and contains the PreS2 domain and the S domain. The PreS2 domain can be about 55 amino acids long and the S-domain can contain about 226 amino acids. An HBV PreS2.S antigen of the application can contain the sequences of any of the naturally occurring PreS2 and S domains, and variants or derivatives thereof. In some embodiments, an internal signal peptide of PreS2.S is left intact to facilitate secretion PreS2.S protein products of the HBV M and HBV S antigens. In one embodiment, an HBV PreS2.S antigen is an HBV M surface antigen. In another embodiment, an HBV PreS2.S antigen is an HBV S surface antigen. In yet another embodiment, an HBV PreS2.S antigen encompasses an HBV M surface antigen and an HBV S surface antigen.

In some embodiments, an HBV surface antigen amino acid sequence is operably linked to or contains a signal peptide for secretion. Any suitable signal peptides can be used. In one embodiment, an HBV Pre-S1 antigen amino acid sequence is operably linked at its N-terminus to a Cystatin S precursor signal peptide to enhance secretion. In a particular embodiment, the Cystatin S precursor signal peptide has the amino acid sequence of SEQ ID NO: 77. In another particular embodiment, a coding sequence of an HBV Pre-S1 antigen is operably linked to a coding sequence of Cystatin S precursor signal peptide having the polynucleotide sequence of SEQ ID NO: 90.

In an embodiment of the application, an HBV antigen comprises an HBV surface antigen, or any immunogenic fragment or combination thereof. An HBV surface antigen is capable of inducing an immune response in a subject against at least one of L-surface antigen, M-surface antigen, and S-surface antigen proteins. Preferably, an HBV surface antigen, such as a Pre-S1 or PreS2.S antigen, is a consensus antigen, preferably a consensus antigen derived from at least two HBV genotypes A, B, C, and D, and more preferably a consensus antigen derived from HBV genotypes A, B, C, and D.

In some embodiments, an HBV surface antigen of the application comprises one or more T cell epitopes for MHC class I HLA alleles. In some embodiments, an HBV surface antigen comprises one or more T cell epitopes selected from the group consisting of HLA-A*11:01 epitopes, HLA-A*24:02 epitopes, and HLA-A*A2402 epitopes. Preferably, an HBV Pre-S1 antigen comprises one or more T cell epitopes selected from the group consisting of HLA-A*11:01 epitopes and HLA-A*24:02 epitopes. More preferably, an HBV Pre-S1 antigen comprises HLA-A*11:01 and HLA-A*24:02 T cell epitopes. More preferably, an HBV Pre-S1 antigen comprises one or more HLA-A*11:01 epitope(s). Preferably, an HBV PreS2.S antigen comprises one or more T cell epitopes selected from the group consisting of HLA-A*11:01 epitopes, HLA-A*24:02 epitopes, and HLA-A*A2402 epitopes. More preferably, an HBV PreS2.S antigen comprises HLA-A*11:01, HLA-A*24:02, and HLA-A*A2402 T cell epitopes. More preferably, an HBV PreS2.S antigen comprises one or more HLA-A*11:01 epitope(s).

In some embodiments of the application, an HBV surface antigen is a Pre-S1 antigen. An exemplary Pre-S1 antigen according to the application comprises an amino acid sequence that is at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 1 or SEQ ID NO: 3. In some embodiments of the application, an HBV surface antigen is a Pre-S2.S antigen. An exemplary Pre-S2.S antigen according to the application comprises an amino acid sequence that is at least 90% identical to SEQ ID NO: 5, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 5.

In a particular embodiment of the application, an HBV surface antigen is a Pre-S1 antigen consisting of the amino acid sequence of SEQ ID NO: 1 or SEQ ID NO: 3. In another particular embodiment, an HBV surface antigen is encoded by a polynucleotide sequence of SEQ ID NO: 2 or SEQ ID NO: 4. In another particular embodiment, an HBV surface antigen is a Pre-S2.S antigen consisting of the amino acid sequence of SEQ ID NO: 5. In another particular embodiment, an HBV surface antigen is encoded by a polynucleotide sequence of SEQ ID NO: 6.

In some embodiments of the application, an HBV surface antigen is an S-surface antigen. An exemplary S-surface antigen according to the application consists of an amino acid sequence that is at least 98% identical to the amino acid sequence of SEQ ID NO: 79, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 79. SEQ ID NO: 81 is an HBV consensus S-surface antigen derived from HBV genotypes A, B, C, and D. In a particular embodiment of the application, an S-surface antigen consists of the amino acid sequence of SEQ ID NO: 79. In another particular embodiment, an HBV surface antigen is encoded by a polynucleotide sequence of SEQ ID NO: 78.

In some embodiments, an HBV surface antigen is M-surface antigen, or any immunogenic fragment or combination thereof. Preferably, the M-surface antigen is a consensus antigen, preferably a consensus antigen derived from at least two, preferably all, of HBV genotypes A, B, C, and D, and more preferably a consensus antigen derived from HBV genotypes A, B, C, and D. Preferably, the M-surface antigen is capable of inducing or eliciting an immune response against M-surface antigen in a subject.

An exemplary M-surface antigen according to the application comprises or consists of an amino acid sequence that is at least 98% identical to the amino acid sequence of SEQ ID NO: 82, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 82. SEQ ID NO: 82 is an HBV consensus M-surface antigen derived from HBV genotypes A, B, C, and D. In a particular embodiment of the application, an M-surface antigen consists of the amino acid sequence of SEQ ID NO: 82.

In some embodiments, an HBV surface antigen is an L-surface antigen, or any immunogenic fragment or combination thereof. Preferably, the L-surface antigen is a consensus antigen, preferably a consensus antigen derived from at least two, preferably all, of HBV genotypes A, B, C, and D, and more preferably a consensus antigen derived from HBV genotypes A, B, C, and D. Preferably, the L-surface antigen is capable of inducing or eliciting an immune response against L-surface antigen in a subject.

An exemplary L-surface antigen according to the application comprises or consists of an amino acid sequence that is at least 98% identical to the amino acid sequence of SEQ ID NO: 83, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 83. SEQ ID NO: 83 is an HBV consensus L-surface antigen derived from HBV genotypes A, B, C, and D. In a particular embodiment of the application, an M-surface antigen consists of the amino acid sequence of SEQ ID NO: 83.

In some embodiments, an HBV surface antigen comprises a portion of any one of the L-, M-, and S-surface antigens, or any combination thereof. For example, an HBV surface antigen can comprise or consist of the N-terminal L-surface antigen domain. An HBV surface antigen can also comprise or consist of the M-surface antigen domain. An HBV surface antigen can also comprise or consist of the N-terminal L-surface antigen domain and the M-surface antigen domain. An HBV surface antigen can also comprise or consist of the N-terminal L-surface antigen domain, the M-surface antigen domain, and a portion of the 5-surface antigen domain.

An exemplary example of such a surface antigen according to the application consists of an amino acid sequence that is at least 98% identical to the amino acid sequence of SEQ ID NO: 81, such as at least 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 81, preferably at least 98% identical to SEQ ID NO: 81, such as at least 98%, 98.5%, 99%, 99.1%, 99.2%, 99.3%, 99.4%, 99.5%, 99.6%, 99.7%, 99.8%, 99.9% or 100% identical to SEQ ID NO: 81. SEQ ID NO: 81 is a consensus antigen derived from HBV genotypes A, B, C, and D, containing the N-terminal L-surface antigen domain, the entire M-surface antigen domain, and a 15-amino acid C-terminal tail from the S-surface antigen domain. In a particular embodiment of the application, an HBV surface antigen consists of the amino acid sequence of SEQ ID NO: 81. In another particular embodiment, an HBV surface antigen is encoded by a polynucleotide sequence of SEQ ID NO: 80 that encodes an HBV surface antigen. In some embodiments, an HBV surface antigen consists of an amino acid sequence, or is encoded by a polynucleotide sequence, described in European Patent Application No. 19180926, the content of which is herein incorporated by reference in its entirety.

### Polynucleotides and Vectors

In another general aspect, the application provides a nucleic acid molecule or nucleic acid combination comprising a non-naturally polynucleotide sequence encoding an HBV antigen according to the application, and a vector comprising the non-naturally occurring nucleic acid. A nucleic acid molecule can comprise any non-naturally occurring polynucleotide sequence encoding an HBV antigen of the application, which can be made using methods known in the art in view of the present disclosure. Preferably, a non-naturally occurring polynucleotide encodes at least one of a truncated HBV core antigen, an HBV polymerase antigen, an HBV Pre-S1 antigen, and an HBV Pre-S2.S antigen of the application. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is RNA.

In an embodiment of the application, a nucleic acid molecule or combination comprises a non-naturally occurring polynucleotide sequence encoding a truncated HBV core antigen consisting of an amino acid sequence that is at least 90% identical to SEQ ID NO: 7, SEQ ID NO: 84, SEQ ID NO: 85 or SEQ ID NO: 86, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, SEQ ID NO: 84, SEQ ID NO: 85 or SEQ ID NO: 86, preferably 98%, 99% or 100% identical to SEQ ID NO: 7, SEQ ID NO: 84, SEQ ID NO: 85 or SEQ ID NO: 86. In a particular embodiment of the application, a non-naturally occurring nucleic acid molecule encodes a truncated HBV core antigen consisting of the amino acid sequence of SEQ ID NO: 7, SEQ ID NO: 84, SEQ ID NO: 85 or SEQ ID NO: 86. Preferably, the truncated HBV core antigen consists of SEQ ID NO: 86.

Examples of polynucleotide sequences of the application encoding a truncated HBV core antigen consisting of the amino acid sequence of SEQ ID NO: 7, SEQ ID NO: 84, SEQ ID NO: 85 or SEQ ID NO: 86 include, but are not limited to, a polynucleotide sequence at least 90% identical to SEQ ID NO: 8, SEQ ID NO: 87, SEQ ID NO: 88 or SEQ ID NO: 89, respectively, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 8, SEQ ID NO: 87, SEQ ID NO: 88 or SEQ ID NO: 89, preferably 98%, 99% or 100% identical to SEQ ID NO: 8, SEQ ID NO: 87, SEQ ID NO: 88 or SEQ ID NO: 89. Exemplary non-naturally occurring nucleic acid molecules encoding a truncated HBV core antigen have the polynucleotide sequence of SEQ ID NO: 8, SEQ ID NO: 87, SEQ ID NO: 88 or SEQ ID NO: 89. Preferably, the molecule encoding a truncated HBV core antigen has the polynucleotide sequence of SEQ ID NO: 89.

In an embodiment of the application, a nucleic acid molecule or combination comprises a non-naturally occurring polynucleotide sequence encoding a HBV polymerase antigen comprising an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9. In a particular embodiment of the application, a non-naturally occurring nucleic acid molecule encodes a HBV polymerase antigen consisting of the amino acid sequence of SEQ ID NO: 9.

Examples of polynucleotide sequences of the application encoding a HBV Pol antigen comprising the amino acid sequence of SEQ ID NO: 9 include, but are not limited to, a polynucleotide sequence at least 90% identical to SEQ ID NO: 10, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 10, preferably 98%, 99% or 100% identical to SEQ ID NO: 10. Exemplary non-naturally occurring nucleic acid molecules encoding a HBV pol antigen have the polynucleotide sequence of SEQ ID NO: 10.

In an embodiment of the application, a nucleic acid molecule or combination comprises a non-naturally occurring polynucleotide sequence encoding a HBV Pre-S1 antigen consisting of an amino acid sequence that is at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. In a particular embodiment of the application, a non-naturally occurring nucleic acid molecule encodes a HBV Pre-S1 antigen consisting the amino acid sequence of SEQ ID NO: 1 or SEQ ID NO: 3.

Examples of polynucleotide sequences of the application encoding a HBV Pre-S1 antigen consisting of the amino acid sequence of SEQ ID NO: 1 or SEQ ID NO: 3 include, but are not limited to, a polynucleotide sequence at least 90% identical to SEQ ID NO: 2 or SEQ ID NO: 4, respectively, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 98%, 99% or 100% identical to SEQ ID NO: 2 or SEQ ID NO: 4. Exemplary non-naturally occurring nucleic acid molecules encoding a HBV Pre-S1 antigen have the polynucleotide sequence of SEQ ID NOs: 2 or 4.

In an embodiment of the application, a nucleic acid molecule or combination comprises a non-naturally occurring polynucleotide sequence encoding a HBV Pre-S2.S antigen consisting of an amino acid sequence that is at least 90% identical to SEQ ID NO: 5, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5, preferably 98%, 99% or 100% identical to SEQ ID NO: 5. In a particular embodiment of the application, a non-naturally occurring nucleic acid molecule encodes a HBV Pre-S2.S antigen consisting the amino acid sequence of SEQ ID NO: 5.

Examples of polynucleotide sequences of the application encoding a HBV Pre-S2.S antigen consisting of the amino acid sequence of SEQ ID NO: 5 include, but are not limited to, a polynucleotide sequence at least 90% identical to SEQ ID NO: 6, respectively, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 6, preferably 98%, 99% or 100% identical to SEQ ID NO: 6. Exemplary non-naturally occurring nucleic acid molecules encoding a HBV Pre-S2.S antigen have the polynucleotide sequence of SEQ ID NO: 6.

In an embodiment of the application, a nucleic acid molecule or combination comprises a non-naturally occurring polynucleotide sequence encoding comprising, from 5′ end to 3′ end: a polynucleotide sequence encoding a first HBV antigen, a first internal ribosome entry sequence (IRES) element or a polynucleotide sequence encoding a first autoprotease peptide, and a polynucleotide sequence encoding a second HBV antigen, wherein at least one of the first and second HBV antigens is an HBV surface antigen. In some embodiments, the non-naturally occurring polynucleotide sequence further comprises, ordered from the 5′- to 3′-end: a second IRES element or a polynucleotide sequence encoding a second autoprotease peptide operably linked to the 3′ end of the polynucleotide sequence encoding the second HBV antigen, and a polynucleotide sequence encoding a third HBV antigen. In some embodiments, the non-naturally occurring polynucleotide sequence further comprises, ordered from the 5′- to 3′-end: a third IRES element or a polynucleotide sequence encoding a third autoprotease peptide operably linked to the 3′ end of the polynucleotide sequence encoding the third HBV antigen, and a polynucleotide sequence encoding a fourth HBV antigen.

In some embodiments, each of the first, second, third and fourth HBV antigens are independently selected from the group consisting of: (i) a first HBV surface antigen comprising, preferably consisting of, an amino acid sequence that is at least 98% identical to the amino acid sequence of SEQ ID NO: 1, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 1; (ii) a second HBV surface antigen comprising, preferably consisting of, an amino acid sequence that is at least 98% identical to the amino acid sequence of SEQ ID NO: 3, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 3; (iii) a third HBV surface antigen comprising, preferably consisting of, an amino acid sequence that is at least 98% identical to the amino acid sequence of SEQ ID NO: 5, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 5; (iv) an HBV core antigen comprising, preferably consisting of, an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7; and (v) an HBV polymerase antigen comprising, preferably consisting of, an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9. In some embodiments, the HBV core antigen comprises, preferably consists of, an amino acid sequence that is at least 90% identical to SEQ ID NO: 84, SEQ ID NO: 85 or SEQ ID NO: 86, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 84, SEQ ID NO: 85 or SEQ ID NO: 86.

In some embodiments, each of the polynucleotide sequences encoding the first, second, third and fourth HBV antigens are independently selected from the group consisting of: (i) a polynucleotide sequence encoding the first HBV PreS1 antigen having a sequence that is at least 90% identical to SEQ ID NO: 2, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2; (ii) a polynucleotide sequence encoding the second HBV PreS1 antigen having a sequence that is at least 90% identical to SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4; (iii) a polynucleotide sequence encoding an HBV PreS2.S antigen having a sequence that is at least 90% identical to SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 6; (iv) a polynucleotide sequence encoding the HBV polymerase antigen having a sequence that is at least 90% identical to SEQ ID NO: 8, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 8; and (v) the polynucleotide sequence encoding the HBV core antigen having a sequence that is at least 90% identical to SEQ ID NO: 10, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 10. In some embodiments, the polynucleotide sequence encoding the HBV core antigen comprises, preferably consists of, an amino acid sequence that is at least 90% identical to SEQ ID NO: 87, SEQ ID NO: 88 or SEQ ID NO: 89, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 87, SEQ ID NO: 88 or SEQ ID NO: 89.

In some embodiments, each of the first, second and third autoprotease peptides independently comprise a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof. Preferably, each of the first, second and third autoprotease peptides comprise the peptide sequence of P2A, such as a P2A sequence of SEQ ID NO: 11. Preferably, the polynucleotide sequence encoding the P2A peptide sequence is SEQ ID NO: 12.

In some embodiments, each of the first, second and third IRES are derived from encephalomyocarditis virus (EMCV) or Enterovirus 71 (EV71), preferably each of the first, second and third IRES comprise the polynucleotide sequence of SEQ ID NO: 13 or 14.

In an embodiment of the application, a nucleic acid molecule or combination comprises a non-naturally occurring polynucleotide sequence encoding comprising, from 5′ end to 3′ end: (1) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen having, preferably consisting of, the amino acid sequence of SEQ ID NO: 9; (2) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen having the amino acid sequence of SEQ ID NO: 7, preferably, consisting of the amino acid sequence of SEQ ID NO: 84, 85 or 86; (3) a polynucleotide sequence encoding an HBV Pre-S1 antigen having, preferably consisting of, the amino acid sequence of SEQ ID NO: 1 or 3, a polynucleotide sequence encoding a P2A amino acid sequence of SEQ ID NO: 11 or an IRES having the polynucleotide sequence of SEQ ID NO: 13 or 14, and a polynucleotide sequence encoding an HBV PreS2.S antigen having, preferably consisting of, the amino acid sequence of SEQ ID NO: 5; (4) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11 or an IRES having the polynucleotide sequence of SEQ ID NO: 13 or 14, and a polynucleotide sequence encoding an HBV Pre-S1 antigen having, preferably consisting of, the amino acid sequence of SEQ ID NO: 1 or 3; (5) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (6) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (7) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV polymerase antigen having, preferably consisting of, the amino acid sequence of SEQ ID NO: 9; (8) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7, preferably, consisting of the amino acid sequence of SEQ ID NO: 84, 85 or 86; (9) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, an IRES having the polynucleotide sequence of SEQ ID NO: 13, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (10) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, an IRES having the polynucleotide sequence of SEQ ID NO: 13, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (11) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, an IRES having the polynucleotide sequence of SEQ ID NO: 13, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV polymerase antigen having, preferably consisting of, the amino acid sequence of SEQ ID NO: 9; (12) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, an IRES having the polynucleotide sequence of SEQ ID NO: 13, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7, preferably, consisting of the amino acid sequence of SEQ ID NO: 84, 85 or 86; (13) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, an IRES having the polynucleotide sequence of SEQ ID NO: 14, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (14) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, an IRES having the polynucleotide sequence of SEQ ID NO: 14, a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, and a polynucleotide sequence encoding an HBV Pre-S1 antigen having, preferably consisting of, the amino acid sequence of SEQ ID NO: 1 or 3; (15) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, an IRES having the polynucleotide sequence of SEQ ID NO: 14, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV polymerase antigen having, preferably consisting of, the amino acid sequence of SEQ ID NO: 9; (16) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV Pre-S1 antigen having, preferably consisting of, the amino acid sequence of SEQ ID NO: 1 or 3, an IRES having the polynucleotide sequence of SEQ ID NO: 14, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7, preferably, consisting of the amino acid sequence of SEQ ID NO: 84, 85 or 86; (17) a polynucleotide sequence encoding an HBV PreS2.S antigen having, preferably consisting of, the amino acid sequence of SEQ ID NO: 5, an IRES having the polynucleotide sequence of SEQ ID NO: 13 or 14, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV core antigen having, preferably consisting of, the amino acid sequence of SEQ ID NO: 86; (18) a polynucleotide sequence encoding an HBV PreS2.S antigen having the amino acid sequence of SEQ ID NO: 5, an IRES having the polynucleotide sequence of SEQ ID NO: 14, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV polymerase antigen having, preferably consisting of, the amino acid sequence of SEQ ID NO: 9; (19) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, an IRES having the polynucleotide sequence of SEQ ID NO: 13 or 14, and a polynucleotide sequence encoding an HBV PreS2.S antigen having, preferably consisting of, the amino acid sequence of SEQ ID NO: 5; (20) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, an IRES having the polynucleotide sequence of SEQ ID NO: 13 or 14, and a polynucleotide sequence encoding an HBV PreS2.S antigen having, preferably consisting of, the amino acid sequence of SEQ ID NO: 5; (21) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7, preferably, consisting of the amino acid sequence of SEQ ID NO: 84, 85 or 86; (22) a polynucleotide sequence encoding an HBV PreS2.S antigen having, preferably consisting of, the amino acid sequence of SEQ ID NO: 5, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV polymerase antigen having, preferably consisting of, the amino acid sequence of SEQ ID NO: 9; (23) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, and a polynucleotide sequence encoding an HBV PreS2.S antigen having, preferably consisting of, the amino acid sequence of SEQ ID NO: 5; and (24) a polynucleotide sequence encoding an HBV core antigen having the amino acid sequence of SEQ ID NO: 7, preferably, consisting of the amino acid sequence of SEQ ID NO: 84, 85 or 86, a polynucleotide sequence encoding a P2A amino acid sequence of SEQ ID NO: 11, a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11, and a polynucleotide sequence encoding an HBV PreS2.S antigen having, preferably consisting of, the amino acid sequence of SEQ ID NO: 5. In some embodiments, each of the HBV antigen is independently operably linked to or contains a signal peptide sequence for secretion. Any suitable signal peptide sequence can be used. Preferably, for each of an HBV PreS1 antigen, an HBV core antigen and an HBV pol antigen, a signal peptide is operably linked to the N-terminus of the antigen sequence. In some embodiments, the signal peptide contains the amino acid sequence of SEQ ID NO: 77, preferably, the signal peptide is encoded by a nucleotide sequence of SEQ ID NO: 90.

In some embodiments, the nucleic acid molecule or nucleic acid combination comprises a non-naturally occurring polynucleotide sequence of any one of SEQ ID NOs: 15 to 54. In some embodiments, the nucleic acid molecule or nucleic acid combination comprises at least two non-naturally occurring polynucleotide sequences of SEQ ID NOs: 15 to 54.

The application also relates to a vector comprising a non-naturally occurring polynucleotide encoding an HBV antigen. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. Preferably, an expression vector is based on pVAX-1, which can be further modified to optimize protein expression in mammalian cells. pVAX-1 is commonly used plasmid in DNA vaccines, and contains a strong human intermediate early cytomegalovirus (CMV-IE) promoter followed by the bovine growth hormone (bGH)-derived polyadenylation sequence (pA). pVAX-1 further contains a pUC origin of replication and kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. In certain embodiments, a vector as described herein is, for instance, a recombinant adenovirus, a recombinant retrovirus, a recombinant pox virus such as a vaccinia virus (e.g., Modified Vaccinia Ankara (MVA)), a recombinant alphavirus such as Semliki forest virus, a recombinant paramyxovirus, such as a recombinant measles virus, or another recombinant virus. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. In certain embodiments, a vector as described herein is an MVA vector. The vector can also be a non-viral vector.

In some embodiments, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding an HBV antigen of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid or a viral vector (particularly an adenoviral vector), can comprise any regulatory elements to establish conventional function(s) of the vector, including but not limited to replication and expression of the HBV antigen(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an HBV antigen within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. Additional promoters suitable for use in the application include, but are not limited to, an RSV promoter, the retrovirus LTR, the adenovirus major late promoter, and various poxvirus promoters including, but not limited to the following vaccinia virus or MVA-derived and FPV-derived promoters: the 30K promoter, the I3 promoter, the PrS promoter, the PrHyb, the PrS5E promoter, the Pr7.5K, the Pr13.5 long promoter, the 40K promoter, the MVA-40K promoter, the FPV 40K promoter, 30k promoter, the PrSynIIm promoter, the PrLE1 promoter, and the PR1238 promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic. Preferably, a promoter is a 26S subgenomic promoter or T7 promoter. A nucleotide sequence of an exemplary 26S subgenomic promoter is shown in SEQ ID NO: 62. A nucleotide sequence of an exemplary T7 promoter is shown in SEQ ID NO: 73.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal (e.g., SEQ ID NO: 64), LTR polyadenylation signal, bovine growth hormone (bGH) polyadenylation signal, human growth hormone (hGH) polyadenylation signal, or human β-globin polyadenylation signal. Preferably, a polyadenylation signal is a SV40 polyadenylation signal. A nucleotide sequence of an exemplary SV40 polyadenylation signal is shown in SEQ ID NO: 64.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as a Cystatin S signal peptide, an Ig heavy chain gamma signal peptide SPIgG, or an Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an HBV antigen, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., HBV antigen. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

The polynucleotides and expression vectors encoding the HBV antigens of the application can be made by any method known in the art in view of the present disclosure. For example, a polynucleotide encoding an HBV antigen can be introduced or “cloned” into an expression vector using standard molecular biology techniques, e.g., polymerase chain reaction (PCR), etc., which are well known to those skilled in the art.

### Adenoviruses

In an aspect, the application provides a recombinant adenovirus comprising a nucleotide sequence encoding an antigenic HBV antigen. In an aspect, the application provides a recombinant MVA vector comprising a nucleotide sequence encoding an antigenic HBV core antigen. In another aspect, the application provides a recombinant MVA vector comprising a nucleotide sequence encoding an antigenic HBV pol antigen. In another aspect, the application provides a recombinant MVA vector comprising a nucleotide sequence encoding an antigenic HBV surface antigen. In an aspect, the application provides a recombinant MVA vector comprising one, two, three, or four nucleotide sequences encoding an antigenic HBV antigen, each independently selected from the group consisting of an HBV core antigen, an HBV polymerase (pol) antigen, and an HBV surface antigen.

An adenovirus according to the application belongs to the family of the Adenoviridae and preferably is one that belongs to the genus Mastadenovirus. It can be a human adenovirus, but also an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu; in the application a human adenovirus is meant if referred to as Ad without indication of species, e.g. the brief notation “Ads” means the same as HAdV5, which is human adenovirus serotype 5), or a simian adenovirus such as chimpanzee or gorilla adenovirus (ChAd, AdCh, or SAdV).

Most advanced studies have been performed using human adenoviruses, and human adenoviruses are preferred according to certain aspects of the application. In certain preferred embodiments, the recombinant adenovirus according to the application is based upon a human adenovirus. In preferred embodiments, the recombinant adenovirus is based upon a human adenovirus serotype 5, 11, 26, 34, 35, 48, 49 or 50. According to a particularly preferred embodiment of the application, an adenovirus is a human adenovirus of one of the serotypes 26 or 35.

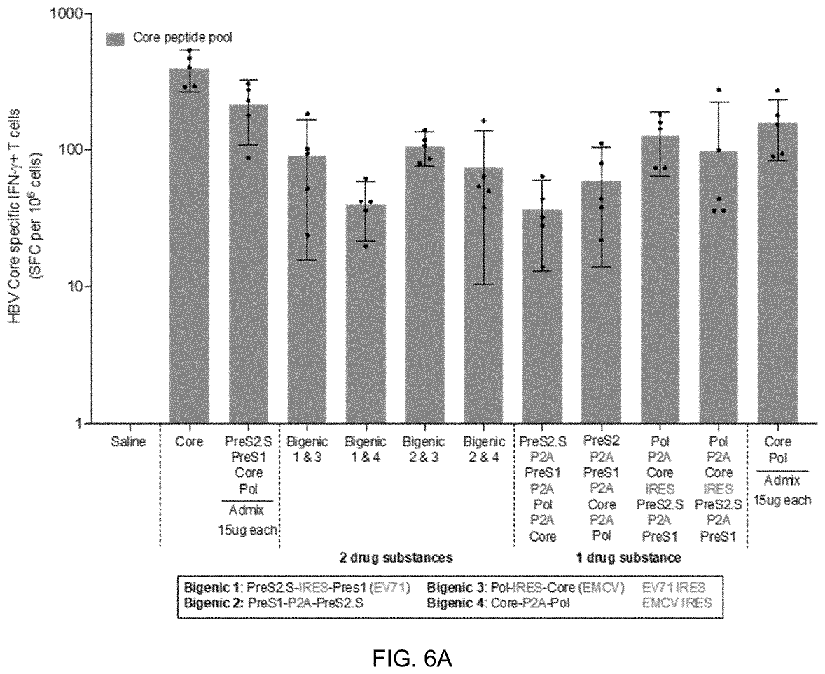

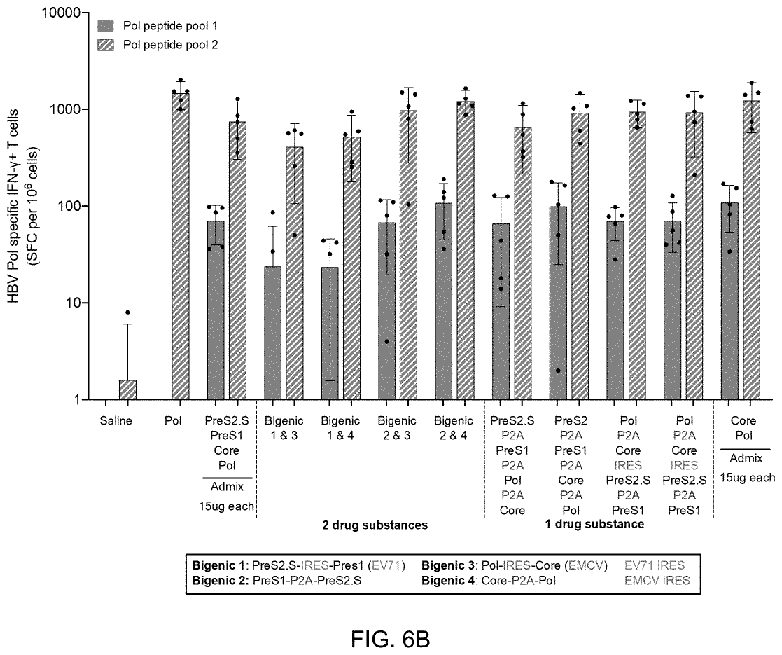

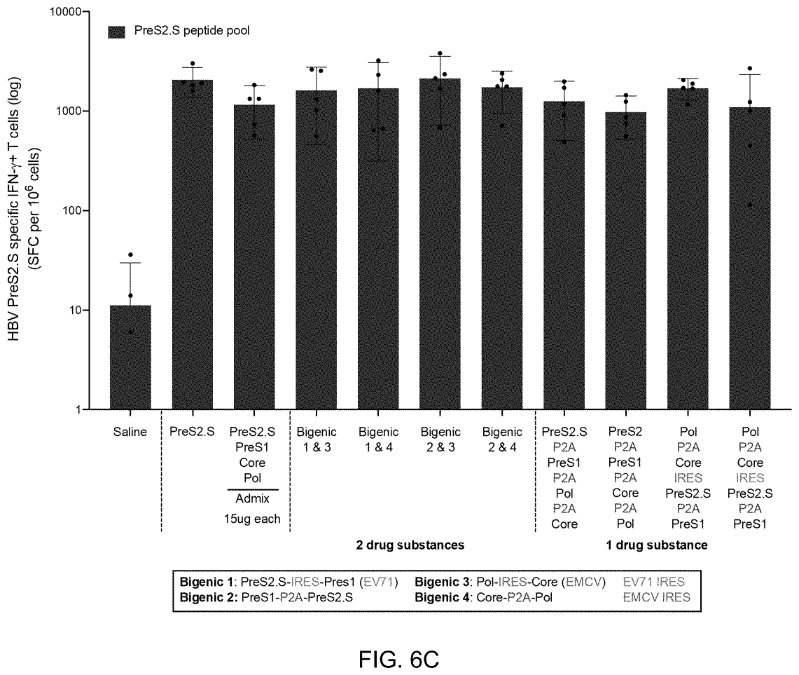

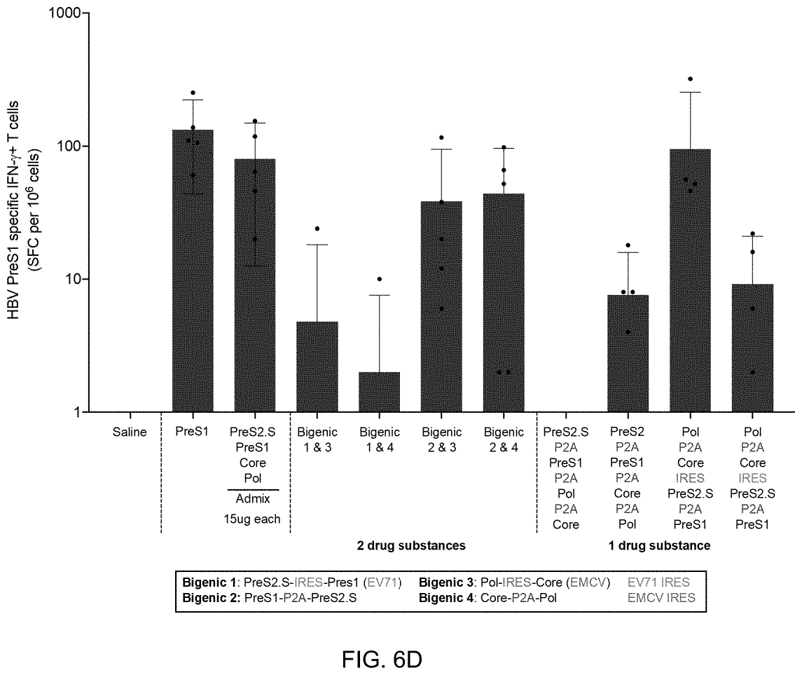

An advantage of these serotypes is a low seroprevalence and/or low pre-existing neutralizing antibody titers in the human population. Preparation of rAd26 vectors is described, for example, in WO 2007/104792 and in Abbink et al., (2007) Virol 81(9): 4654-63, both of which are incorporated by reference herein in their entirety. Exemplary genome sequences of Ad26 are found in GenBank Accession EF 153474 and in WO2007/104792 (see, e.g., SEQ ID NO:1). Preparation of rAd35 vectors is described, for example, in U.S. Pat. No. 7,270,811, in WO00/70071, and in Vogels et al., (2003) J Virol 77(15): 8263-71, all of which are incorporated by reference herein in their entirety. Exemplary genome sequences of Ad35 are found in GenBank Accession AC_000019 and in WO00/70071 (see, e.g., FIG. 6).

Simian adenoviruses generally also have a low seroprevalence and/or low pre-existing neutralizing antibody titers in the human population, and a significant amount of work has been reported using chimpanzee adenovirus vectors (e.g. U.S. Pat. No. 6,083,716; WO2005/071093; WO 2010/086189; WO 2010085984; Farina et al, 2001, J Virol 75: 11603-13; Cohen et al, 2002, J Gen Virol 83: 151-55; Kobinger et al, 2006, Virology 346: 394-401; Tatsis et al., 2007, Molecular Therapy 15: 608-17; see also review by Bangari and Mittal, 2006, Vaccine 24: 849-62; and review by Lasaro and Ertl, 2009, Mol Ther 17: 1333-39). Hence, in other preferred embodiments, the recombinant adenovirus according to the application is based upon a simian adenovirus, e.g. a chimpanzee adenovirus. In an embodiment of the application, the recombinant adenovirus is based upon simian adenovirus type 1, 3, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P.

Adenoviral Vectors rAd26 and rAd35

In a preferred embodiment of the application, the adenoviral vectors comprise capsid proteins from two rare serotypes: Ad26 and Ad35. In the typical embodiment, the vector is an rAd26 or rAd35 virus.

Thus, the vectors that can be used in the application comprise an Ad26 or Ad35 capsid protein (e.g., a fiber, penton or hexon protein). One of skill will recognize that it is not necessary that an entire Ad26 or Ad35 capsid protein be used in the vectors of the application. Thus, chimeric capsid proteins that include at least a part of an Ad26 or Ad35 capsid protein can be used in the vectors of the application. The vectors of the application may also comprise capsid proteins in which the fiber, penton, and hexon proteins are each derived from a different serotype, so long as at least one capsid protein is derived from Ad26 or Ad35. In preferred embodiments, the fiber, penton and hexon proteins are each derived from Ad26 or each from Ad35.

One of skill will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the application could combine the absence of pre-existing immunity of the Ad26 and Ad35 serotypes with characteristics such as temperature stability, assembly, anchoring, production yield, redirected or improved infection, stability of the DNA in the target cell, and the like.

In an embodiment of the application the recombinant adenovirus vector useful in the application is derived mainly or entirely from Ad35 or from Ad26 (i.e., the vector is rAd35 or rAd26). In some embodiments, the adenovirus is replication deficient, e.g. because it contains a deletion in the E1 region of the genome. For the adenoviruses of the application, being derived from Ad26 or Ad35, it is typical to exchange the E4-orf6 coding sequence of the adenovirus with the E4-orf6 of an adenovirus of human subgroup C, such as Ad5. This allows propagation of such adenoviruses in well-known complementing cell lines that express the E1 genes of Ad5, such as for example 293 cells, PER.C6 cells, and the like (see, e.g. Havenga et al, 2006, J Gen Virol 87: 2135-43; WO 03/104467). In an embodiment of the application, the adenovirus is a human adenovirus of serotype 35, with a deletion in the E1 region into which the nucleic acid encoding the antigen has been cloned, and with an E4 orf6 region of Ad5. In an embodiment of the application, the adenovirus is a human adenovirus of serotype 26, with a deletion in the E1 region into which the nucleic acid encoding the antigen has been cloned, and with an E4 orf6 region of Ad5. For the Ad35 adenovirus, it is typical to retain the 3′ end of the E1B 55K open reading frame in the adenovirus, for instance the 166 bp directly upstream of the pIX open reading frame or a fragment comprising this such as a 243 bp fragment directly upstream of the pIX start codon, marked at the 5′ end by a Bsu36I restriction site, since this increases the stability of the adenovirus because the promoter of the pIX gene is partly residing in this area (see, e.g. Havenga et al, 2006, supra; WO 2004/001032).

The preparation of recombinant adenoviral vectors is well known in the art. Preparation of rAd26 vectors is described, for example, in WO 2007/104792 and in Abbink et al., (2007) Virol 81(9): 4654-63. Exemplary genome sequences of Ad26 are found in GenBank Accession EF 153474 and in SEQ ID NO:1 of WO 2007/104792. Preparation of rAd35 vectors is described, for example, in U.S. Pat. No. 7,270,811 and in Vogels et al., (2003) J Virol 77(15): 8263-71. An exemplary genome sequence of Ad35 is found in GenBank Accession AC_000019.

In an embodiment of the application, the vectors useful in the application include those described in WO2012/082918, the disclosure of which is incorporated herein by reference in its entirety.

Typically, a vector useful in the application is produced using a nucleic acid comprising the entire recombinant adenoviral genome (e.g., a plasmid, cosmid, or baculovirus vector). Thus, the application also provides isolated nucleic acid molecules that encode the adenoviral vectors of the application. The nucleic acid molecules of the application may be in the form of RNA or in the form of DNA obtained by cloning or produced synthetically. The DNA may be double-stranded or single-stranded.

The adenovirus vectors useful in the application are typically replication defective. In these embodiments, the virus is rendered replication-defective by deletion or inactivation of regions critical to replication of the virus, such as the E1 region. The regions can be substantially deleted or inactivated by, for example, inserting the gene of interest (usually linked to a promoter). In some embodiments, the vectors of the application may contain deletions in other regions, such as the E2, E3 or E4 regions or insertions of heterologous genes linked to a promoter. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amount of adenovirus vectors of the application. A packaging cell is a cell that comprises those genes that have been deleted or inactivated in a replication-defective vector, thus allowing the virus to replicate in the cell. Suitable cell lines include, for example, PER.C6, 911, 293, and E1 A549.

As noted above, a wide variety of Hepatitis B virus (HBV) antigens (e.g., HBV core, HBV polymerase, HBV Pre-S1, HBV PreS2.S antigens) can be expressed in the vectors. If required, the heterologous gene encoding the HBV antigen can be codon-optimized to ensure proper expression in the treated host (e.g., human). Codon-optimization is a technology widely applied in the art. Typically, the heterologous gene is cloned into the E1 and/or the E3 region of the adenoviral genome.

The heterologous Hepatitis B virus gene may be under the control of (i.e., operably linked to) an adenovirus-derived promoter (e.g., the Major Late Promoter) or may be under the control of a heterologous promoter. Examples of suitable heterologous promoters include the CMV promoter and the RSV promoter. Preferably, the promoter is located upstream of the heterologous gene of interest within an expression cassette.

### MVA Vectors

MVA vectors useful for the application utilize attenuated virus derived from Modified Vaccinia Ankara virus. The MVA vectors express a wide variety of HBV antigens (e.g., HBV core, HBV polymerase, HBV Pre-S1, HBV PreS2.S antigens). In an aspect, the application provides a recombinant MVA vector comprising a nucleotide sequence encoding an antigenic HBV core antigen. In another aspect, the application provides a recombinant MVA vector comprising a nucleotide sequence encoding an antigenic HBV pol antigen. In another aspect, the application provides a recombinant MVA vector comprising a nucleotide sequence encoding an antigenic HBV surface antigen. In an aspect, the application provides a recombinant MVA vector comprising one, two, three, or four nucleotide sequences encoding an antigenic HBV antigen, each independently selected from the group consisting of an HBV core antigen, an HBV polymerase (pol) antigen, and an HBV surface antigen.

The man-made attenuated modified vaccinia virus Ankara (“MVA”) was generated by 516 serial passages on chicken embryo fibroblasts of the Ankara strain of vaccinia virus (CVA) (for review see Mayr, A., et al. Infection 3, 6-14 (1975)). As a consequence of these long-term passages, the genome of the resulting MVA virus had about 31 kilobases of its genomic sequence deleted and, therefore, was described as highly host cell restricted for replication to avian cells (Meyer, H. et al., J. Gen. Virol. 72, 1031-1038 (1991)). It was shown in a variety of animal models that the resulting MVA was significantly a virulent compared to the fully replication competent starting material (Mayr, A. & Danner, K., Dev. Biol. Stand. 41: 225-34 (1978)).

An MVA virus useful in the practice of the application can include, but is not limited to, MVA-572 (deposited as ECACC V94012707 on Jan. 27, 1994); MVA-575 (deposited as ECACC V00120707 on Dec. 7, 2000), MVA-1721 (referenced in Suter et al., Vaccine 2009), and ACAM3000 (deposited as ATCC® PTA-5095 on Mar. 27, 2003).

More preferably the MVA used in accordance with the application includes MVA-BN and derivatives of MVA-BN. MVA-BN has been described in International PCT publication WO 02/42480. “Derivatives” of MVA-BN refer to viruses exhibiting essentially the same replication characteristics as MVA-BN, as described herein, but exhibiting differences in one or more parts of their genomes.

MVA-BN, as well as derivatives thereof, is replication incompetent, meaning a failure to reproductively replicate in vivo and in vitro. More specifically in vitro, MVA-BN or derivatives thereof have been described as being capable of reproductive replication in chicken embryo fibroblasts (CEF), but not capable of reproductive replication in the human keratinocyte cell line HaCat (Boukamp et al (1988), J. Cell Biol. 106:761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or derivatives thereof have a virus amplification ratio at least two fold less, more preferably three-fold less than MVA-575 in Hela cells and HaCaT cell lines. Tests and assay for these properties of MVA-BN and derivatives thereof are described in WO 02/42480 (U.S. Patent application No. 2003/0206926) and WO 03/048184 (U.S. Patent application No. 2006/0159699).

The term “not capable of reproductive replication” or “no capability of reproductive replication” in human cell lines in vitro as described in the previous paragraphs is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or in U.S. Pat. No. 6,761,893.

The term “failure to reproductively replicate” refers to a virus that has a virus amplification ratio in human cell lines in vitro as described in the previous paragraphs at 4 days after infection of less than 1. Assays described in WO 02/42480 or in U.S. Pat. No. 6,761,893 are applicable for the determination of the virus amplification ratio.

The amplification or replication of a virus in human cell lines in vitro as described in the previous paragraphs is normally expressed as the ratio of virus produced from an infected cell (output) to the amount originally used to infect the cell in the first place (input) referred to as the “amplification ratio”. An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

The advantages of MVA-based vaccine include their safety profile as well as availability for large scale vaccine production. Preclinical tests have revealed that MVA-BN demonstrates superior attenuation and efficacy compared to other MVA strains (WO 02/42480). An additional property of MVA-BN strains is the ability to induce substantially the same level of immunity in vaccinia virus prime/vaccinia virus boost regimes when compared to DNA-prime/vaccinia virus boost regimes.

The recombinant MVA-BN viruses, the most preferred embodiment herein, are considered to be safe because of their distinct replication deficiency in mammalian cells and their well-established avirulence. Furthermore, in addition to its efficacy, the feasibility of industrial scale manufacturing can be beneficial. Additionally, MVA-based vaccines can deliver multiple heterologous antigens and allow for simultaneous induction of humoral and cellular immunity.

MVA vectors useful for the application can be prepared using methods known in the art, such as those described in WO/2002/042480 and WO/2002/24224, the relevant disclosures of which are incorporated herein by references.

In a preferred embodiment of the application, the MVA vector(s) comprise a nucleic acid that encodes one or more antigenic proteins selected from the group consisting of HBV core antigen, HBV pol antigen, and HBV surface antigens.

The HBV antigen protein may be inserted into one or more intergenic regions (IGR) of the MVA. In an embodiment of the application, the IGR is selected from IGR07/08, IGR 44/45, IGR 64/65, IGR 88/89, IGR 136/137, and IGR 148/149. In an embodiment of the application, less than 5, 4, 3, or 2 IGRs of the recombinant MVA comprise heterologous nucleotide sequences encoding antigenic determinants of a HBV core antigen and/or a HBV pol antigen. The heterologous nucleotide sequences may, additionally or alternatively, be inserted into one or more of the naturally occurring deletion sites, in particular into the main deletion sites I, II, III, W, V, or VI of the MVA genome. In an embodiment of the application, less than 5, 4, 3, or 2 of the naturally occurring deletion sites of the recombinant MVA comprise heterologous nucleotide sequences encoding antigenic determinants of a HBV core antigen and/or a HBV pol antigen.

The number of insertion sites of MVA comprising heterologous nucleotide sequences encoding antigenic determinants of a HBV protein can be 1, 2, 3, 4, 5, 6, 7, or more. In an embodiment of the application, the heterologous nucleotide sequences are inserted into 4, 3, 2, or fewer insertion sites. Preferably, two insertion sites are used. In an embodiment of the application, three insertion sites are used. Preferably, the recombinant MVA comprises at least 2, 3, 4, 5, 6, or 7 genes inserted into 2 or 3 insertion sites.

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual (2nd Ed.) (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of MVA are described in Molecular Virology: A Practical Approach (A. J. Davison & R. M. Elliott (Eds.), The Practical Approach Series, IRL Press at Oxford University Press, Oxford, UK (1993)(see, e.g., Chapter 9: Expression of genes by Vaccinia virus vectors)) and Current Protocols in Molecular Biology (John Wiley & Son, Inc. (1998)(see, e.g., Chapter 16, Section IV: Expression of proteins in mammalian cells using vaccinia viral vector)).

For the generation of the various recombinant MVAs disclosed herein, different methods may be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter.

Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome, such as MVA, cloned as a bacterial artificial chromosome (BAC) and a linear foreign sequence flanked with DNA sequences homologous to sequences flanking the desired site of integration in the vaccinia virus genome.

The heterologous HBV gene (e.g., an HBV core antigen, an HBV pol antigen, and/or an HBV surface antigen) may be under the control of (i.e., operably linked to) one or more poxvirus promoters. In an embodiment of the application, the poxvirus promoter is a Pr7.5 promoter, a hybrid early/late promoter, or a PrS promoter, a PrS5E promoter, a synthetic or natural early or late promoter, or a cowpox virus ATI promoter.

### RNA Replicons

Preferably, the vector is a self-replicating RNA replicon. As used herein, “self-replicating RNA molecule,” which is used interchangeably with “self-amplifying RNA molecule” or “RNA replicon” or “replicon RNA” or “saRNA,” refers to RNA which contains all of the genetic information required for directing its own amplification or self-replication within a permissive cell, which can be a human, mammalian, or animal cell. A self-replicating RNA molecule resembles mRNA. It is single-stranded, 5′-capped, and 3′-poly-adenylated and is of positive orientation. To direct its own replication, the RNA molecule 1) encodes polymerase, replicase, or other proteins which can interact with viral or host cell-derived proteins, nucleic acids or ribonucleoproteins to catalyze the RNA amplification process; and 2) contain cis-acting RNA sequences required for replication and transcription of the subgenomic replicon-encoded RNA. Thus, the delivered RNA leads to the production of multiple daughter RNAs. These daughter RNAs, as well as collinear subgenomic transcripts, can be translated themselves to provide in situ expression of a gene of interest, or can be transcribed to provide further transcripts with the same sense as the delivered RNA which are translated to provide in situ expression of the gene of interest. The overall result of this sequence of transcriptions is a huge amplification in the number of the introduced replicon RNAs and so the encoded gene of interest becomes a major polypeptide product of the cells.

The RNA replicon 1) encodes an RNA-dependent RNA polymerase, which may interact with viral or host cell-derived proteins, nucleic acids or ribonucleoproteins to catalyze the RNA amplification process, and the non-structural proteins nsP1, nsP2, nsP3, nsP4; and 2) contains cis-acting RNA sequences required for replication and transcription of the genomic and subgenomic RNAs, such as 3′ and 5′ untranslated regions (U IRs; alphavirus nucleotide sequences for non-structural protein-mediated amplification), and/or a subgenomic promoter. These sequences can be bound during the process of replication to self-encoded proteins, or non-self-encoded cell-derived proteins, nucleic acids or ribonucleoproteins, or complexes between any of these components. In some embodiments, a modified RNA replicon molecule typically contains the following ordered elements: 5′ viral RNA sequence(s) required in cis for replication (e.g. a 5′ UTR and a 5′ CSE), sequences coding for biologically active nonstructural proteins (e.g. nsP1234), a promoter for transcribing the subgenomic RNA, 3′ viral sequences required in cis for replication (e.g. 3′ UTR), and a polyadenylate tract, and optionally, a sequence (or two or more sequences) encoding a heterologous protein or peptide after or under the control of a sub-genomic promoter. Further, the term RNA replicon can refer to a positive sense (or message sense) molecule and the RNA replicon can be of a length different from that of any known, naturally-occurring RNA viruses. In any of the embodiments of the present disclosure, the RNA replicon can lack (or not contain) the sequence(s) of at least one (or all) of the structural viral proteins (e.g. nucleocapsid protein C, and envelope proteins P62, 6K, and E1). In these embodiments, the sequences encoding one or more structural genes can be substituted with one or more heterologous sequences such as, for example, a coding sequence for at least one heterologous protein or peptide (or other gene of interest (GOI)).

In certain embodiments, an RNA replicon of the application comprises, ordered from the 5′- to 3′-end: (1) a 5′ untranslated region (5′-UTR) required for nonstructural protein-mediated amplification of an RNA virus; (2) a polynucleotide sequence encoding at least one, preferably all, of non-structural proteins of the RNA virus; (3) a subgenomic promoter of the RNA virus; (4) a polynucleotide sequence encoding an HBV antigen; and (5) a 3′ untranslated region (3′-UTR) required for nonstructural protein-mediated amplification of the RNA virus.

In certain embodiments, a self-replicating RNA molecule encodes an enzyme complex for self-amplification (replicase polyprotein) comprising an RNA-dependent RNA-polymerase function, helicase, capping, and poly-adenylating activity. The viral structural genes downstream of the replicase, which are under control of a subgenomic promoter, can be replaced by an HBV antigen. Upon transfection, the replicase is translated immediately, interacts with the 5′ and 3′ termini of the genomic RNA, and synthesizes complementary genomic RNA copies. Those act as templates for the synthesis of novel positive-stranded, capped, and poly-adenylated genomic copies, and subgenomic transcripts. Amplification eventually leads to very high RNA copy numbers of up to 2×10⁵ copies per cell. Thus, much lower amounts of saRNA compared to conventional mRNA suffice to achieve effective gene transfer and protective vaccination (Beissert et al., Hum Gene Ther. 2017, 28(12): 1138-1146).

Subgenomic RNA is an RNA molecule of a length or size which is smaller than the genomic RNA from which it was derived. The viral subgenomic RNA can be transcribed from an internal promoter, whose sequences reside within the genomic RNA or its complement. Transcription of a subgenomic RNA can be mediated by viral-encoded polymerase(s) associated with host cell-encoded proteins, ribonucleoprotein(s), or a combination thereof. Numerous RNA viruses generate subgenomic mRNAs (sgRNAs) for expression of their 3′-proximal genes.

In some embodiments of the present disclosure, an HBV antigen is expressed under the control of a subgenomic promoter. In certain embodiments, instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71 (EV71), or hepatitis C viruses. Subgenomic promoters range from 24 nucleotide (Sindbis virus) to over 100 nucleotides (Beet necrotic yellow vein virus) and are usually found upstream of the transcription start.

In some embodiments, the RNA replicon includes the coding sequence for at least one, at least two, at least three, or at least four nonstructural viral proteins (e.g. nsP1, nsP2, nsP3, nsP4). Alphavirus genomes encode non-structural proteins nsP1, nsP2, nsP3, and nsP4, which are produced as a single polyprotein precursor, sometimes designated P1234 (or nsP1-4 or nsP1234), and which is cleaved into the mature proteins through proteolytic processing. nsP1 can be about 60 kDa in size and may have methyltransferase activity and be involved in the viral capping reaction. nsP2 has a size of about 90 kDa and may have helicase and protease activity while nsP3 is about 60 kDa and contains three domains: a macrodomain, a central (or alphavirus unique) domain, and a hypervariable domain (HVD). nsP4 is about 70 kDa in size and contains the core RNA-dependent RNA polymerase (RdRp) catalytic domain. After infection the alphavirus genomic RNA is translated to yield a P1234 polyprotein, which is cleaved into the individual proteins. In disclosing the nucleic acid or polypeptide sequences herein, for example sequences of nsP1, nsP2, nsP3, nsP4, also disclosed are sequences considered to be based on or derived from the original sequence.

In some embodiments, RNA replicon includes the coding sequence for a portion of the at least one nonstructural viral protein. For example, the RNA replicon can include about 10%, 20%, 30%, 40%, 50%, 60%, 70%, 80%, 90%, 95%, 100%, or a range between any two of these values, of the encoding sequence for the at least one nonstructural viral protein. In some embodiments, the RNA replicon can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). In some embodiments, the RNA replicon can include the entire coding sequence for the at least one nonstructural protein. In some embodiments, the RNA replicon comprises substantially all the coding sequence for the native viral nonstructural proteins. In certain embodiments, the one or more nonstructural viral proteins are derived from the same virus. In other embodiments, the one or more nonstructural proteins are derived from different viruses.

The RNA replicon can be derived from any suitable plus-strand RNA viruses, such as alphaviruses or flaviviruses. Preferably, the RNA replicon is derived from alphaviruses. The term “alphavirus” describes enveloped single-stranded positive sense RNA viruses of the family Togaviridae. The genus alphavirus contains approximately 30 members, which can infect humans as well as other animals. Alphavirus particles typically have a 70 nm diameter, tend to be spherical or slightly pleomorphic, and have a 40 nm isometric nucleocapsid. The total genome length of alphaviruses ranges between 11,000 and 12,000 nucleotides and has a 5′cap and 3′ poly-A tail. There are two open reading frames (ORF's) in the genome, non-structural (ns) and structural. The ns ORF encodes proteins (nsP1-nsP4) necessary for transcription and replication of viral RNA. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome.

In some embodiments, the self-replicating RNA useful for the invention is an RNA replicon derived from an alphavirus virus species. In some embodiments, the alphavirus RNA replicon is of an alphavirus belonging to the VEEV/EEEV group, or the SF group, or the SIN group. Non-limiting examples of SF group alphaviruses include Semliki Forest virus, O'Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S. A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

Non-limiting examples of alphavirus species include Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (STNV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. Virulent and a virulent alphavirus strains are both suitable. In some embodiments, the alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV). In some embodiments, the alphavirus RNA replicon is of a Venezuelan equine encephalitis virus (VEEV).

In certain embodiments, a self-replicating RNA molecule comprises a polynucleotide encoding one or more nonstructural proteins nsP1-4, a subgenomic promoter, such as 26S subgenomic promoter, and a gene of interest encoding an HBV antigen or a fragment thereof described herein.

A self-replicating RNA molecule can have a 5′ cap (e.g. a 7-methylguanosine). This cap can enhance in vivo translation of the RNA.

The 5′ nucleotide of a self-replicating RNA molecule useful with the invention can have a 5′ triphosphate group. In a capped RNA this can be linked to a 7-methylguanosine via a 5′-to-5′ bridge. A 5′ triphosphate can enhance RIG-I binding.

A self-replicating RNA molecule can have a 3′ poly-A tail. It can also include a poly-A polymerase recognition sequence (e.g. AAUAAA) near its 3′ end.

In any of the embodiments of the present disclosure, the RNA replicon can lack (or not contain) the coding sequence(s) of at least one (or all) of the structural viral proteins (e.g. nucleocapsid protein C, and envelope proteins P62, 6K, and E1). In these embodiments, the sequences encoding one or more structural genes can be substituted with one or more heterologous sequences such as, for example, a coding sequence for an HBV antigen or a fragment thereof described herein.

In certain embodiments, a self-replicating RNA vector of the application comprises one or more features to confer a resistance to the translation inhibition by the innate immune system or to otherwise increase the expression of the GOI (e.g., an HBV antigen).

In certain embodiments, the RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription.

In certain embodiments, an RNA replicon of the application comprises, ordered from the 5′- to 3′-end, (1) an alphavirus 5′ untranslated region (5′-UTR), (2) a 5′ replication sequence of an alphavirus non-structural gene nsp1, (3) a downstream loop (DLP) motif of a virus species, (4) a polynucleotide sequence encoding a fourth autoprotease peptide, (5) a polynucleotide sequence encoding alphavirus non-structural proteins nsp1, nsp2, nsp3 and nsp4, (6) an alphavirus subgenomic promoter, (7) the non-naturally occurring polynucleotide sequence encoding one or more HBV antigens of the application, (8) an alphavirus 3′ untranslated region (3′ UTR), and (9) optionally, a poly adenosine sequence.

In certain embodiments, a self-replicating RNA vector of the application comprises a downstream loop (DLP) motif of a virus species. As used herein, a “downstream loop” or “DLP motif” refers to a polynucleotide sequence comprising at least one RNA stem-loop, which when placed downstream of a start codon of an open reading frame (ORF) provides increased translation the ORF compared to an otherwise identical construct without the DLP motif. As an example, members of the Alphavirus genus can resist the activation of antiviral RNA-activated protein kinase (PKR) by means of a prominent RNA structure present within in viral 26S transcripts, which allows an eIF2-independent translation initiation of these mRNAs. This structure, called the downstream loop (DLP), is located downstream from the AUG in S1NV 26S mRNA. The DLP is also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The predicted structures of these Alphavirus 26S mRNAs were constructed based on SHAPE (selective 2′-hydroxyl acylation and primer extension) data (Toribio et al., Nucleic Acids Res. May 19; 44(9):4368-80, 2016), the content of which is hereby incorporated by reference). Stable stem-loop structures were detected in all cases except for CHIKV and ONNV, whereas MAYV and EEEV showed DLPs of lower stability (Toribio et al., 2016 supra). In the case of Sindbis virus, the DLP motif is found in the first 150 nt of the Sindbis subgenomic RNA. The hairpin is located downstream of the Sindbis capsid AUG initiation codon (AUG is collated at nt 50 of the Sindbis subgenomic RNA). Previous studies of sequence comparisons and structural RNA analysis revealed the evolutionary conservation of DLP in SINV and predicted the existence of equivalent DLP structures in many members of the Alphavirus genus (see e.g., Ventoso, J. Virol. 9484-9494, Vol. 86, September 2012). Examples of a self-replicating RNA vector comprising a DLP motif are described in US Patent Application Publication US2018/0171340 and the International Patent Application Publication WO2018106615, the content of which is incorporated herein by reference in its entirety. In some embodiments, a replicon RNA of the application comprises a DLP motif exhibiting at least 90%, at least 95%, at least 96%, at least 97%, at least 98%, at least 99%, or 100% sequence identity to the sequences set forth in SEQ ID NO: 57.

In one embodiment, the self-replicating RNA molecule also contains a coding sequence for an autoprotease peptide operably linked downstream of the DLP motif and upstream of the coding sequences of the nonstructural proteins (e.g., one or more of nsp1-4) or gene of interest (e.g., an HBV antigen described herein). Examples of the autoprotease peptide include, but are not limited to, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, a replicon RNA of the application comprises a coding sequence for P2A having the amino acid sequence of SEQ ID NO: 11. Preferably, the coding sequence exhibits at least 90%, at least 95%, at least 96%, at least 97%, at least 98%, at least 99%, or 100% sequence identity to the sequences set forth in SEQ ID NO: 12.

Any of the replicons of the invention can also comprise a 5′ and a 3′ untranslated region (UTR). The UTRs can be wild type New World or Old World alphavirus UTR sequences, or a sequence derived from any of them. In various embodiments the 5′ UTR can be of any suitable length, such as about 60 nt or 50-70 nt or 40-80 nt. In some embodiments the 5′ UTR can also have conserved primary or secondary structures (e.g. one or more stem-loop(s)) and can participate in the replication of alphavirus or of replicon RNA. In some embodiments the 3′ UTR can be up to several hundred nucleotides, for example it can be 50-900 or 100-900 or 50-800 or 100-700 or 200 nt-700 nt. The ‘3 UTR also can have secondary structures, e.g. a step loop, and can be followed by a polyadenylate tract or poly-A tail. In any of the embodiments of the invention the 5’ and 3′ untranslated regions can be operably linked to any of the other sequences encoded by the replicon. The UTRs can be operably linked to a promoter and/or sequence encoding a heterologous protein or peptide by providing sequences and spacing necessary for recognition and transcription of the other encoded sequences. Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal.

In another embodiment, a self-replicating RNA replicon of the application comprises a modified 5′ untranslated region (5′-UTR), preferably the RNA replicon is devoid of at least a portion of a nucleic acid sequence encoding viral structural proteins. For example, the modified 5′-UTR can comprise one or more nucleotide substitutions at position 1, 2, 4, or a combination thereof. Preferably, the modified 5′-UTR comprises a nucleotide substitution at position 2, more preferably, the modified 5′-UTR has a U->G or U->A substitution at position 2. Examples of such self-replicating RNA molecules are described in US Patent Application Publication US2018/0104359 and the International Patent Application Publication WO2018075235, the content of which is incorporated herein by reference in its entirety. In some embodiments, a replicon RNA of the application comprises a 5′-UTR exhibiting at least 90%, at least 95%, at least 96%, at least 97%, at least 98%, at least 99%, or 100% sequence identity to the sequences set forth in SEQ ID NO: 55.

In some embodiments, an RNA replicon of the application comprises, ordered from the 5′- to 3′-end, (1) a 5′-UTR having the polynucleotide sequence of SEQ ID NO: 55, (2) a 5′ replication sequence having the polynucleotide sequence of SEQ ID NO: 56, (3) a DLP motif comprising the polynucleotide sequence of SEQ ID NO: 57, (4) a polynucleotide sequence encoding a P2A sequence of SEQ ID NO: 11, (5) polynucleotide sequences encoding alphavirus non-structural proteins nsp1, nsp2, nsp3 and nsp4, having the nucleic acid sequences of SEQ ID NO: 58, SEQ ID NO: 59, SEQ ID NO: 60 and SEQ ID NO: 61, respectively, (6) a subgenomic promoter having polynucleotide sequence of SEQ ID NO: 62, (7) a non-naturally occurring polynucleotide sequence as described herein, and (8) a 3′ UTR having the polynucleotide sequence of SEQ ID NO: 63. In some embodiments, the polynucleotide sequence encoding the P2A sequence comprises SEQ ID NO: 12, the non-naturally occurring polynucleotide sequence comprises the polynucleotide sequence of any one of SEQ ID NOs: 15 to 54, and the RNA replicon further comprises a poly adenosine sequence. Preferably the poly adenosine sequence has the sequence of SEQ ID NO: 64, at the 3′-end of the replicon.

In some preferred embodiments, an RNA replicon of the application comprises the polynucleotide sequence of any one of SEQ ID NOs: 65 to 72.

In some embodiments, an RNA replicon of the application comprises a polynucleotide sequence encoding a signal peptide sequence. Preferably, the polynucleotide sequence encoding the signal peptide sequence is located upstream of or at the 5′-end of the polynucleotide sequence encoding an HBV antigen, such as an HBV PreS1 antigen, an HBV core antigen and an HBV pol antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the replicon, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as a Cystatin S signal peptide, an Ig heavy chain gamma signal peptide SPIgG, an Ig heavy chain epsilon signal peptide SPIgE, or a short leader peptide sequence. An exemplary amino acid sequence of a signal peptide is shown in SEQ ID NO: 77.

In various embodiments the RNA replicons disclosed herein can be engineered, synthetic, or recombinant RNA replicons. As used herein, the term recombinant means any molecule (e.g. DNA, RNA, etc.), that is or results, however indirectly, from human manipulation of a polynucleotide. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant RNA replicon can be one or more of the following: 1) synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) conjoined nucleotide sequences that are not conjoined in nature; 3) engineered using molecular cloning techniques such that it lacks one or more nucleotides with respect to the naturally occurring nucleotide sequence; and 4) manipulated using molecular cloning techniques such that it has one or more sequence changes or rearrangements with respect to the naturally occurring nucleotide sequence.

Any of the components or sequences of the RNA replicon can be operably linked to any other of the components or sequences. The components or sequences of the RNA replicon can be operably linked for the expression of at least one heterologous protein or peptide (or biotherapeutic) in a host cell or treated organism and/or for the ability of the replicon to self-replicate. The term “operably linked” denotes a functional linkage between two or more sequences that are configured so as to perform their usual function. Thus, a promoter or UTR operably linked to a coding sequence is capable of effecting the transcription and expression of the coding sequence when the proper enzymes are present. The promoter need not be contiguous with the coding sequence, so long as it functions to direct the expression thereof. Thus, an operable linkage between an RNA sequence encoding a heterologous protein or peptide and a regulatory sequence (for example, a promoter or UTR) is a functional link that allows for expression of the polynucleotide of interest. Operably linked can also refer to sequences such as the sequences encoding nsP1-4, the UTRs, promoters, and other sequences encoding in the RNA replicon, are linked so that they enable transcription and translation of the biotherapeutic molecule and/or replication of the replicon. The UTRs can be operably linked by providing sequences and spacing necessary for recognition and translation by a ribosome of other encoded sequences.

The RNA replicons of the invention can be derived from alphavirus genomes, meaning that they have some of the structural characteristics of alphavirus genomes, or be similar to them. The RNA replicons of the invention can be modified alphavirus genomes. In some embodiments of the replicons disclosed herein one or more sequences of the replicon can be provided “in trans,” i.e. the sequences of the replicon are provided on more than one RNA molecule. In other embodiments all of the sequences of the replicon are present on a single RNA molecule, which can also be administered to a mammal to be treated as described herein.

As used herein, the terms “percent identity” or “homology” or “shared sequence identity” or “percent (%) sequence identity” with respect to nucleic acid or polypeptide sequences are defined as the percentage of nucleotide or amino acid residues in the candidate sequence that are identical with the known polynucleotides or polypeptides, after aligning the sequences for maximum percent identity and introducing gaps, if necessary, to achieve the maximum percent homology. N-terminal or C-terminal insertions or deletions shall not be construed as affecting homology, and internal deletions and/or insertions into the nucleotide or polypeptide sequence of less than about 30, less than about 20, or less than about 10 or less than 5 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A BESTFIT® comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

In disclosing the nucleic acid or polypeptide sequences herein, for example sequences of viral capsid enhancers, autoprotease peptides, subgenomic promoters, nonstructural proteins, HBV antigens, also disclosed are sequences considered to be based on or derived from the original sequence. Sequences disclosed therefore include polynucleotide or polypeptide sequences having sequence identities of at least 40%, at least 45%, at least 50%, at least 55%, of at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or 100% or 85-99% or 85-95% or 90-99% or 95-99% or 97-99% or 98-99% sequence identity with the full-length polynucleotide or polypeptide sequence of any polynucleotide or polypeptide sequence described herein, respectively, such as SEQ ID NOs: 1-90, and fragments thereof. Also disclosed are fragments or portions of any of the sequences disclosed herein. Fragments or portions of sequences can include sequences having at least 5 or at least 7 or at least 10, or at least 20, or at least 30, at least 50, at least 75, at least 100, at least 125, 150 or more or 5-10 or 10-12 or 10-15 or 15-20 or 20-40 or 20-50 or 30-50 or 30-75 or 30-100 amino acid or nucleic acid residues of the entire sequence, or at least 100 or at least 200 or at least 300 or at least 400 or at least 500 or at least 600 or at least 700 or at least 800 or at least 900 or at least 1000 or 100-200 or 100-500 or 100-1000 or 500-1000 amino acid or nucleic acid residues, or any of these amounts but less than 500 or less than 700 or less than 1000 or less than 2000 consecutive amino acids or nucleic acids of any of SEQ ID NOs: 1-90 or of any fragment disclosed herein. Also disclosed are variants of such sequences, e.g., where at least one or two or three or four or five amino acid residues have been inserted N- and/or C-terminal to, and/or within, the disclosed sequence(s) which contain(s) the insertion and substitution, and nucleic acid sequences encoding such variants. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme). The nucleic acid sequences described herein can be RNA sequences.

### Heterologous Proteins and Peptides

The RNA replicons of the invention can include an RNA sequence encoding at least one protein or peptide that is heterologous to an alphavirus and can also be (but is not necessarily) heterologous to the human, mammal, or animal that expresses the RNA sequence in the body. In any embodiment the replicons can have RNA sequence(s) encoding two or three or four or more heterologous proteins or peptides. In some embodiments, the heterologous protein or peptide is an HBV antigen as described herein. In any of the embodiments the sequence encoding the heterologous protein or peptide can be operably linked to one or more other sequences of the replicon (e.g. a promoter or 5′ or 3′ UTR sequences), and can be under the control of a sub-genomic promoter so that the heterologous protein or peptide is expressed in the human, mammal, or animal.

In one embodiment the RNA replicon of the invention can have an RNA sequence encoding a heterologous protein or peptide (e.g. a monoclonal antibody or a biotherapeutic protein or peptide), RNA sequences encoding amino acid sequences derived from wild type alphavirus nsP1, nsP2, nsP3, and nsP4 protein sequences, and 5′ and 3′ UTR sequences (for non-structural protein-mediated amplification). The RNA replicons can also have a 5′ cap and a poly adenylate (or poly-A) tail.

The immunogenicity of a heterologous protein or peptide can be determined by a number of assays known to persons of ordinary skill, for example immunostaining of intracellular cytokines or secreted cytokines by epitope-specific T-cell populations, or by quantifying frequencies and total numbers of epitope-specific T-cells and characterizing their differentiation and activation state, e.g., short-lived effector and memory precursor effector CD8+ T-cells. Immunogenicity can also be determined by measuring an antibody-mediated immune response, e.g. the production of antibodies by measuring serum IgA or IgG titers.

In addition to the HBV antigens of the application, the RNA replicons of the application can optionally further encode one or more heterologous proteins or peptides that can be any protein or peptide, including but not limited to, cytokines, growth factors, immunoglobulins, monoclonal antibodies (including Fab antigen-binding fragments, Fc fusion proteins), hormones, interferons, interleukins, regulatory peptides and proteins.

In some embodiments the heterologous protein or peptide can be encoded by an RNA sequence of up to 5 kb or up to 6 kb or up to 7 kb or up to 8 kb, or up to 9 kb or up to 10 kb or up to 11 kb or up to 12 kb. The heterologous protein can also be a single-chain antibody molecule.

The alphavirus replicons of the invention can also have a sub-genomic promoter for expression of the heterologous protein or peptide. The term “subgenomic promoter,” as used herein, refers to a promoter of a subgenomic mRNA of a viral nucleic acid. As used herein, an “alphavirus subgenomic promoter” is a promoter as originally defined in an alphavirus genome that directs transcription of a subgenomic messenger RNA as part of the alphavirus replication process.

The term “heterologous” when used in reference to a polynucleotide, a gene, a nucleic acid, a polypeptide, a protein, or an enzyme, refers to a polynucleotide, gene, a nucleic acid, polypeptide, protein, or an enzyme that is not derived from the host species. For example, “heterologous gene” or “heterologous nucleic acid sequence” as used herein, refers to a gene or nucleic acid sequence from a different species than the species of the host organism it is introduced into. Heterologous sequences can also be synthetic and not derived from an organism or not found in Nature. When referring to a gene regulatory sequence or to an auxiliary nucleic acid sequence used for manipulating expression of a gene sequence (e.g. a 5′ untranslated region, 3′ untranslated region, poly A addition sequence, intron sequence, splice site, ribosome binding site, internal ribosome entry sequence, genome homology region, recombination site, etc.) or to a nucleic acid sequence encoding a protein domain or protein localization sequence, “heterologous” means that the regulatory or auxiliary sequence or sequence encoding a protein domain or localization sequence is from a different source than the gene with which the regulatory or auxiliary nucleic acid sequence or nucleic acid sequence encoding a protein domain or localization sequence is juxtaposed in a genome, chromosome or episome. Thus, a promoter operably linked to a gene to which it is not operably linked to in its natural state (for example, in the genome of a non-genetically engineered organism) is referred to herein as a “heterologous promoter,” even though the promoter may be derived from the same species (or, in some cases, the same organism) as the gene to which it is linked. Similarly, when referring to a protein localization sequence or protein domain of an engineered protein, “heterologous” means that the localization sequence or protein domain is derived from a protein different from that into which it is incorporated by genetic engineering.

The term “non-naturally occurring,” “recombinant” or “engineered” nucleic acid molecule or polynucleotide sequence, as used herein, refers to a nucleic acid molecule or non-naturally occurring polynucleotide sequence that has been altered through human intervention. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector or that has been integrated into an RNA replicon.

In some embodiments, an RNA replicon of the invention comprises, ordered from the 5′- to 3′-end: a 5′ untranslated region (5′-UTR) required for nonstructural protein-mediated amplification of an RNA virus; a polynucleotide sequence encoding at least one, preferably all, of non-structural proteins of the RNA virus; a subgenomic promoter of the RNA virus; a non-naturally occurring polynucleotide sequence described herein; and a 3′ untranslated region (3′-UTR) required for nonstructural protein-mediated amplification of the RNA virus.

In some embodiments, an RNA replicon of the invention comprises, ordered from the 5′- to 3′-end: an alphavirus 5′ untranslated region (5′-UTR); a 5′ replication sequence of an alphavirus non-structural gene nsp1; a downstream loop (DLP) motif of a virus species; a polynucleotide sequence encoding a fourth autoprotease peptide; a polynucleotide sequence encoding alphavirus non-structural proteins nsp1, nsp2, nsp3 and nsp4; an alphavirus subgenomic promoter; a non-naturally occurring polynucleotide sequence described herein; an alphavirus 3′ untranslated region (3′ UTR); and, optionally, a poly adenosine sequence.

In some embodiments, the DLP motif is from a virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MTDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (U AV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus.

In some embodiments, the fourth autoprotease peptide is selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof. Preferably, the fourth autoprotease peptide comprises the peptide sequence of P2A. In some embodiments, the fourth autoprotease peptide comprises SEQ ID NO: 11. In some embodiments, a polynucleotide sequence encoding a fourth autoprotease peptide comprises SEQ ID NO: 12. In some embodiments, a polynucleotide sequence encoding a fourth autoprotease peptide consists of SEQ ID NO: 12.

In some embodiments, an RNA replicon of the invention comprises, ordered from the 5′- to 3′-end: a 5′-UTR having the polynucleotide sequence of SEQ ID NO: 55; a 5′ replication sequence having the polynucleotide sequence of SEQ ID NO: 56; a DLP motif comprising the polynucleotide sequence of SEQ ID NO: 57; a polynucleotide sequence encoding a P2A sequence of SEQ ID NO: 11; polynucleotide sequences encoding alphavirus non-structural proteins nsp1, nsp2, nsp3 and nsp4, as those encoded by the nucleic acid sequences of SEQ ID NO: 58, SEQ ID NO: 59, SEQ ID NO: 60 and SEQ ID NO: 61, respectively; a subgenomic promoter having polynucleotide sequence of SEQ ID NO: 62; a non-naturally occurring polynucleotide sequence disclosed herein; and a 3′ UTR having the polynucleotide sequence of SEQ ID NO: 63. In some embodiments, the polynucleotide sequence encoding the P2A sequence comprises SEQ ID NO: 12, the non-naturally occurring polynucleotide sequence comprises the polynucleotide sequence of any one of SEQ ID NOs: 15 to 54, and the RNA replicon further comprises a poly adenosine sequence. Preferably, the poly adenosine sequence has the sequence of SEQ ID NO: 64 at the 3′-end of the replicon.

In some embodiments, an RNA replicon of the invention comprises the polynucleotide sequence of any one of SEQ ID NOs: 65 to 72.

In some embodiments, a nucleic acid molecule comprising a polynucleotide sequence encoding an RNA replicon disclosed herein further comprises a T7 promoter operably linked to the 5′-end of the DNA sequence. More preferably, the T7 promoter comprises the nucleotide sequence of SEQ ID NO: 73.

Also provided are methods of producing an RNA replicon of the application, comprising transcribing a nucleic acid molecule comprising a DNA sequence encoding a RNA replicon disclosed herein. In some embodiments, the nucleic acid molecule is transcribed in vivo. In some embodiments, the nucleic acid molecule is transcribed in vitro.

### Cells and Polypeptides

The application also provides cells, preferably isolated cells, comprising any of the polynucleotides and vectors described herein. The cells can, for instance, be used for recombinant protein production, or for the production of viral particles. In some embodiments, the cells can be used for production of an RNA replicon.

Host cells comprising a RNA replicon or a nucleic acid encoding the RNA replicon of the application also form part of the invention. The HBV antigens may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells, such as human cells, or insect cells. In general, the production of a recombinant protein, such the HBV antigens of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, polyA sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)). Cell culture media are available from various vendors, and a suitable medium can be routinely chosen for a host cell to express the protein of interest, here the HBV antigens. The suitable medium may or may not contain serum.

Embodiments of the application thus also relate to a method of making an HBV antigen of the application. The method comprises transfecting a host cell with an expression vector comprising a polynucleotide encoding an HBV antigen of the application operably linked to a promoter, growing the transfected cell under conditions suitable for expression of the HBV antigen, and optionally purifying or isolating the HBV antigen expressed in the cell. The HBV antigen can be isolated or collected from the cell by any method known in the art including affinity chromatography, size exclusion chromatography, etc. Techniques used for recombinant protein expression will be well known to one of ordinary skill in the art in view of the present disclosure. The expressed HBV antigens can also be studied without purifying or isolating the expressed protein, e.g., by analyzing the supernatant of cells transfected with an expression vector encoding the HBV antigen and grown under conditions suitable for expression of the HBV antigen.

Thus, also provided are non-naturally occurring or recombinant polypeptides comprising an amino acid sequence that is at least 90% identical to the amino acid sequence of SEQ ID NO: 1, SEQ ID NO: 3, SEQ ID NO: 5, SEQ ID NO: 7, 84, 85 or 86, or SEQ ID NO: 9. As described above and below, isolated nucleic acid molecules encoding these sequences, vectors comprising these sequences operably linked to a promoter, and compositions comprising the polypeptide, polynucleotide, or vector are also contemplated by the application.

In an embodiment of the application, a recombinant polypeptide comprises an amino acid sequence that is at least 90% identical to the amino acid sequence of SEQ ID NO: 1, SEQ ID NO: 3, SEQ ID NO: 5, SEQ ID NO: 7, 84, 85 or 86, or SEQ ID NO: 9, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1, SEQ ID NO: 3, SEQ ID NO: 5, SEQ ID NO: 7, 84, 85 or 86, or SEQ ID NO: 9. Preferably, a non-naturally occurring or recombinant polypeptide consists of SEQ ID NO: 1, SEQ ID NO: 3, SEQ ID NO: 5, SEQ ID NO: 84, 85 or 86, or SEQ ID NO: 9.

### Compositions

The application also relates to compositions, pharmaceutical compositions, immunogenic combinations, and more particularly vaccines, comprising one or more HBV antigens, polynucleotides, and/or vectors encoding one more HBV antigens according to the application. Any of the HBV antigens, polynucleotides (including RNA and DNA), and/or vectors of the application described herein can be used in the compositions, pharmaceutical compositions, immunogenic combinations, and vaccines of the application.

The application provides, for example, a pharmaceutical composition comprising any nucleic acid molecule, vector, or RNA replicon described herein, together with a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Pharmaceutical compositions of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Pharmaceutical compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, pharmaceutical compositions of the application are formulated for parental injection, preferably subcutaneous, intradermal injection, or intramuscular injection, more preferably intramuscular injection.

According to embodiments of the application, pharmaceutical compositions for administration will typically comprise a buffered solution in a pharmaceutically acceptable carrier, e.g., an aqueous carrier such as buffered saline and the like, e.g., phosphate buffered saline (PBS). The compositions and immunogenic combinations can also contain pharmaceutically acceptable substances as required to approximate physiological conditions such as pH adjusting and buffering agents. For example, a pharmaceutical composition of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL, 1 mg/mL, 2 mg/mL, 3 mg/mL, 4 mg/mL, or 5 mg/mL, preferably at 1 mg/mL.

In some embodiments, a pharmaceutical composition of the application comprising an RNA replicon can be administered in a concentration of, e.g., about 20 μg/mL to about 200 μg/mL, such as 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, or 200 μg/mL. In some embodiments, a pharmaceutical composition of the application comprising an RNA replicon can be administered in a concentration below 20 μg/mL. In some embodiments, a pharmaceutical composition of the application comprising an RNA replicon can be administered in a concentration above 200 μg/mL.

Pharmaceutical compositions of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In a particular embodiment of the application, a pharmaceutical composition, composition, or immunogenic combination is a DNA vaccine. DNA vaccines typically comprise bacterial plasmids containing a polynucleotide encoding an antigen of interest under control of a strong eukaryotic promoter. Once the plasmids are delivered to the cell cytoplasm of the host, the encoded antigen is produced and processed endogenously. The resulting antigen typically induces both humoral and cell-medicated immune responses. DNA vaccines are advantageous at least because they offer improved safety, are temperature stable, can be easily adapted to express antigenic variants, and are simple to produce. Any of the DNA plasmids of the application can be used to prepare such a DNA vaccine.

In other particular embodiments of the application, a pharmaceutical compositions, composition, or immunogenic combination is an RNA vaccine. RNA vaccines typically comprise at least one single-stranded RNA molecule encoding an antigen of interest, e.g., HBV antigen. Once the RNA is delivered to the cell cytoplasm of the host, the encoded antigen is produced and processed endogenously, inducing both humoral and cell-mediated immune responses, similar to a DNA vaccine. The RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription. An RNA vaccine can also be self-replicating RNA vaccine developed from an alphavirus expression vector. Self-replicating RNA vaccines comprise a replicase RNA molecule derived from a virus belonging to the alphavirus family with a subgenomic promoter that controls replication of the HBV antigen RNA followed by an artificial poly A tail located downstream of the replicase.

In certain embodiments, an adjuvant is included in a pharmaceutical composition of the application, or co-administered with a pharmaceutical composition of the application. Use of an adjuvant is optional, and can further enhance immune responses when the composition is used for vaccination purposes. Adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL-12 genetic adjuvant, and IL-7-hyFc.

The application also provides methods of making pharmaceutical compositions and immunogenic combinations of the application. A method of producing a pharmaceutical composition or immunogenic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

### Methods of Inducing an Immune Response

The application also provides methods of inducing an immune response against hepatitis B virus (HBV) in a subject in need thereof, comprising administering to the subject an immunogenically effective amount of a pharmaceutical composition of the application. Any of the pharmaceutical compositions of the application described herein can be used in the methods of the application.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

The application also provides methods of vaccinating a subject against HBV, comprising administering to the subject a pharmaceutical composition of the application. In some embodiments, the vaccination of a subject is prophylactic vaccination or therapeutic vaccination, more particularly the vaccination is therapeutic vaccination. The application also provides methods for reducing infection and/or replication of HBV in a subject, comprising administering to the subject a pharmaceutical composition of the application or a vaccine of the application. Any of the pharmaceutical compositions or vaccines of the application described herein can be used in the methods of the application.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of pharmaceutical compositions and immunogenic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof. An immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or immunogenic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

As general guidance, an immunogenically effective amount when used with reference to a nucleic acid molecule, vector, or RNA replicon can range from about 1 μg of nucleic acid molecule, vector, or RNA replicon to about 1 mg of nucleic acid molecule, vector, or RNA replicon, such as 1 μg, 10 μs, 20 μs, 30 μg, 40 μs, 50 μs, 60 μs, 70 μs, 80 μg, 90 μg, 100 μg, 200 μg, 300 μg, 400 μg, 500 μg, 600 μg, 700 μg, 800 μg, 900 μg, or 1 mg. Preferably, an immunogenically effective amount of a nucleic acid molecule, vector, or RNA replicon is about 10 μg to about 100 μg. An immunogenically effective amount when used with reference to a nucleic acid molecule, vector, or RNA replicon in a pharmaceutical composition can range from a concentration of about 0.01 mg/mL to about 2 mg/mL of a nucleic acid molecule, vector, or RNA replicon total, such as 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.25 mg/mL, 0.5 mg/mL, 0.75 mg/mL, 1 mg/mL, 1.5 mg/mL, or 2 mg/mL. Preferably, an immunogenically effective amount of a nucleic acid molecule, vector, or RNA replicon is less than 1 mg/mL, more preferably less than 0.05 mg/mL. An immunogenically effective amount can be from one nucleic acid molecule, vector, or RNA replicon, or from multiple nucleic acid molecules, vectors, or RNA replicons. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. For example, when two DNA plasmids are used, an immunogenically effective amount can be 3-4 mg/mL, with 1.5-2 mg/mL of each plasmid. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

An immunogenic combination comprising two vectors, e.g., a first vector encoding a first HBV antigen and second vector encoding a second HBV antigen can be administered to a subject by mixing both vectors and delivering the mixture to a single anatomic site. Alternatively, two separate immunizations each delivering a single expression vector can be performed. In such embodiments, whether both vectors are administered in a single immunization as a mixture of in two separate immunizations, the first vector and the second vector can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second vectors are administered in a ratio of 1:1, by weight.

Preferably, a subject to be treated according to the methods of the application is an HBV-infected subject, particularly a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that may follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart. Preferably, an immunogenically effective amount refers to the amount of a composition or immunogenic combination of the application which is sufficient to treat chronic HBV infection.

In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV may allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprises administering to the subject in need thereof another immunogenic agent (such as another HBV antigen or other antigen) or another anti-HBV agent (such as a nucleoside analog or other anti-HBV agent) in combination with a pharmaceutical composition of the application. For example, another anti-HBV agent or immunogenic agent can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL-12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen binding fragment thereof, a polypeptide, protein, or nucleic acid.

### Methods of Delivery

Pharmaceutical compositions and immunogenic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, pharmaceutical compositions and immunogenic combinations are administered parenterally (e.g., by intramuscular injection or intradermal injection) or transdermally.

In some embodiments of the application in which a pharmaceutical composition or immunogenic combination comprises one or more DNA plasmids, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA vaccines has been shown to significantly increase plasmid uptake by host cells, while also leading to mild-to-moderate inflammation at the injection site. As a result, transfection efficiency and immune response are significantly improved (e.g., up to 1,000 fold and 100 fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a pharmaceutical composition, immunogenic combination or vaccine of the application via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and immunogenic combinations of the application, particularly those comprising DNA plasmids, include CELLECTRA® (Inovio Pharmaceuticals, Blue Bell, Pa.), Elgen electroporator (Inovio Pharmaceuticals, Inc.) Tri-Grid™ delivery system (Ichor Medical Systems, Inc., San Diego, Calif. 92121) and those described in U.S. Pat. Nos. 7,664,545, 8,209,006, 9,452,285, 5,273,525, 6,110,161, 6,261,281, 6,958,060, and 6,939,862, 7,328,064, 6,041,252, 5,873,849, 6,278,895, 6,319,901, 6,912,417, 8,187,249, 9,364,664, 9,802,035, 6,117,660, and International Patent Application Publication WO2017172838, all of which are herein incorporated by reference in their entireties. Other examples of in vivo electroporation devices are described in International Patent Application entitled “Method and Apparatus for the Delivery of Hepatitis B Virus (HBV) Vaccines,” filed on the same day as this application with the Attorney Docket Number 688097-405WO, the contents of which are hereby incorporated by reference in their entireties. Also contemplated by the application for delivery of the compositions and immunogenic combinations of the application are use of a pulsed electric field, for instance as described in, e.g., U.S. Pat. No. 6,697,669, which is herein incorporated by reference in its entirety.

In other embodiments of the application in which a pharmaceutical composition or immunogenic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or immunogenic combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, lipoplexes, nanoparticles, etc. For example, an RNA replicon of the application can be formulated in an immunogenic composition that comprises one or more lipid molecules, preferably positively charged lipid molecules. In some embodiments, an RNA replicon of the disclosure can be formulated using one or more liposomes, lipoplexes, and/or lipid nanoparticles. In some embodiments, liposome or lipid nanoparticle formulations described herein can comprise a polycationic composition. In some embodiments, the formulations comprising a polycationic composition can be used for the delivery of the RNA replicon described herein in vivo and/or ex vitro.

According to the present invention, the term “lipid” refers to any fatty acid derivative or other amphiphilic compound which is capable of forming a lyotropic lipid phase, or more preferentially, a lamellar lyotropic phase. In particular, the term “lipid” refers to any fatty acid derivative which is capable of forming a bilayer such that a hydrophobic part of the lipid molecule orients toward the bilayer while a hydrophilic part orients toward the aqueous phase. The term “lipid” comprises neutral, anionic or cationic lipids. Lipids preferably comprise a hydrophobic domain with at least one, preferably two, alkyl chains or a cholesterol moiety and a polar head group. The alkyl chains of the fatty acids in the hydrophobic domain of the lipid are not limited to a specific length or number of double bonds. Nevertheless, it is preferred that the fatty acid has a length of 10 to 30, preferably 14 to 25 carbon atoms. The lipid may also comprise two different fatty acids.

In the context of the present disclosure, a lipid-based delivery vehicle typically serves to transport a desired RNA replicon to a target cell or tissue. In some embodiments, the lipid-based delivery vehicle comprises a nanoparticle or a bilayer of lipid molecules and an RNA replicon of the present disclosure. In some embodiments, the lipid bilayer preferably further comprises a neutral lipid or a polymer. The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more RNA replicons encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the RNA replicon or combination of nucleic acid molecules is fully encapsulated within the lipid portion of the lipid formulation such that the RNA replicon or combination of nucleic acid molecules in the lipid formulation is resistant in aqueous solution to nuclease degradation. The term “fully encapsulated” means that the nucleic acid (e.g., RNA replicon) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” as used herein also means that the nucleic acid-lipid particles do not rapidly decompose into their component parts upon in vivo administration. In other embodiments, the lipid formulations described herein are substantially non-toxic to mammals such as humans. In some embodiments, the combination of nucleic acids is encapsulated within the same lipid nanoparticle. In some embodiments, each nucleic acid molecule in the combination of nucleic acid molecules is independently encapsulated in individual lipid nanoparticles.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1. In some preferred embodiments, the total lipid:RNA ratio (mass/mass ratio) is from about 10:1 to about 25:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise an RNA replicon or combination of nucleic acid molecules, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. The term “lipid conjugate” means a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers, and mixtures thereof. PEG or POZ can be conjugated directly to the lipid or may be linked to the lipid via a linker moiety. Any linker moiety suitable for coupling the PEG or the POZ to a lipid can be used including, e.g., non-ester-containing linker moieties and ester-containing linker moieties. In certain preferred embodiments, non-ester-containing linker moieties, such as amides or carbamates, are used. In certain preferred embodiments, the PEG-lipid conjugate is 2-[(polyethylene glycol)-2000]-N,N-ditetradecylacetamide (i.e., ALC-0159).

The term “cationic lipid” as used herein refers to amphiphilic lipids and salts thereof having a positive, hydrophilic head group; one, two, three, or more hydrophobic (i.e., having apolar groups) fatty acid or fatty alkyl chains; and a connector between these two domains. An ionizable or protonatable cationic lipid is typically protonated (i.e., positively charged) at a pH below its pKₐ and is substantially neutral at a pH above the pKₐ. Preferred ionizable cationic lipids are those having a pKₐ that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include C₁₈ alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, (4-hydroxybutyl)azanediyl)bis(hexane-6,1-diyl)bis(2-hexyldecanoate (also known as ALC-0315), Lipofectin™ also known as DOTMA (N-D-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine), DSDMA, DODMA, DLinDMA, DLenDMA, γ-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2K-DMA, DLin-M-C2-DMA (also known as MC2), DLin-M-C3-DMA (also known as MC3), (DLin-MP-DMA)(also known as 1-Bl 1), and cholesterol derivatives such as DCChol (3 beta-(N—(N′,N′-dimethyl aminomethane)-carbamoyl) cholesterol). In certain preferred embodiments, the cationic lipid is ((4-hydroxybutyl)azanediyl)bis(hexane-6,1-diyl)bis(2-hexyldecanoate), i.e., ALC-0315.

The term “anionic lipid” as used herein refers to a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

In the nucleic acid-lipid formulations, the RNA replicon or combination of nucleic acid molecules may be fully encapsulated within the lipid portion of the formulation, thereby protecting the nucleic acid from nuclease degradation. In preferred embodiments, a lipid formulation comprising an RNA replicon or combination of nucleic acid molecules is fully encapsulated within the lipid portion of the lipid formulation, thereby protecting the nucleic acid from nuclease degradation. In certain instances, the RNA replicon or combination of nucleic acid molecules in the lipid formulation is not substantially degraded after exposure of the particle to a nuclease at 37° C. for at least 20, 30, 45, or 60 minutes. In certain other instances, the RNA replicon or combination of nucleic acid molecules in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the RNA replicon or combination of nucleic acid molecules is complexed with the lipid portion of the formulation.

In the context of nucleic acids, full encapsulation may be determined by performing a membrane-impermeable fluorescent dye exclusion assay, which uses a dye that has enhanced fluorescence when associated with a nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where I and I₀ refer to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of RNA replicon-liposomes. In some embodiments, the nucleic acid-lipid composition comprises a plurality of RNA replicon-cationic liposomes. In some embodiments, the nucleic acid-lipid composition comprises a plurality of RNA replicon-lipid nanoparticles.

In some embodiments, the lipid formulations comprise an RNA replicon or combination of nucleic acid molecules that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA replicon or combination of nucleic acid molecules encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the expressible polynucleotides and RNA replicons described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:

(a) an RNA replicon or combination of nucleic acid molecules of the present disclosure,

(b) a cationic lipid,

(c) an aggregation reducing agent (such as polyethylene glycol (PEG) lipid or PEG-modified lipid),

(d) optionally a non-cationic lipid (such as a neutral lipid), and

(e) optionally, a sterol.

Preferably, the lipid nanoparticle encapsulating the RNA replicon or combination of nucleic acid molecules comprises a cationic lipid and at least one other lipid selected from the group consisting of anionic lipids, zwitterionic lipids, neutral lipids, steroids, polymer conjugated lipids, phospholipids, glycolipids, and combinations thereof.

In some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 30% to about 60% ionizable cationic lipid: about 5% to about 20% helper lipid: about 35% to about 50% sterol: about 0.5-5% PEG-lipid. Example cationic lipids (including ionizable cationic lipids), helper lipids (e.g., neutral lipids), sterols, and ligand-containing lipids (e.g., PEG-lipids) are described herein below.

The selection of specific lipids and their relative % compositions depends on several factors including the desired therapeutic effect, the intended in vivo delivery target, and the planned dosing regimen and frequency. Generally, lipids that correspond to both high potency (i.e., therapeutic effect such as knockdown activity or translation efficiency) and biodegradability resulting in rapid tissue clearance are most preferred. However, biodegradability may be less important for formulations that are intended for only one or two administrations within the subject. In addition, the lipid composition may require careful engineering so that the lipid formulation preserves its morphology during in vivo administration and its journey to the intended target, but will then be able to release the active agent upon uptake into target cells. Thus, several formulations typically need to be evaluated in order to find the best possible combination of lipids in the best possible molar ratio of lipids as well as the ratio of total lipid to active ingredient.

Suitable lipid components and methods of manufacturing lipid nanoparticles are well known in the art and are described for example in PCT/US2020/023442, U.S. Pat. Nos. 8,058,069, 8,822,668, 9,738,593, 9,139,554, PCT/US2014/066242, PCT/US2015/030218, PCT/2017/015886, and PCT/US2017/067756, the contents of which are incorporated by reference.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, ((4-hydroxybutyl)azanediyl)bis(hexane-6,1-diyl)bis(2-hexyldecanoate) (also known as ALC-0315), N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dime thylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

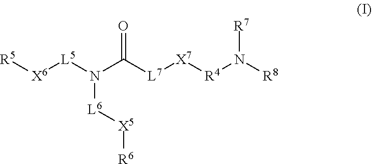

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of CH((CH₂)ₚCH₃)((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is —C(O)O—, whereby —C(O)O—R⁶ is formed, X⁶ is —C(O)O— whereby —C(O)O—R⁵ is formed, L⁵ and L⁶ are each independently a linear C₃-C₇ alkyl, L⁷ is absent, R⁵ is —CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group consisting of

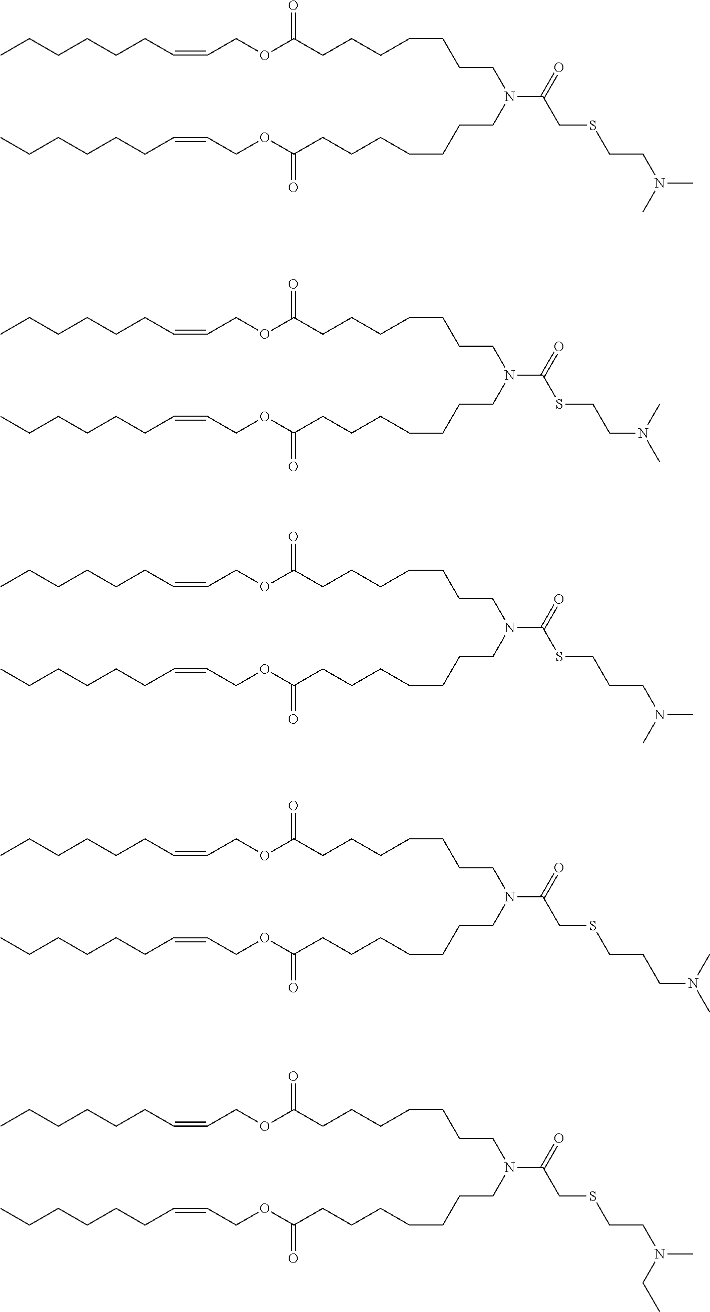

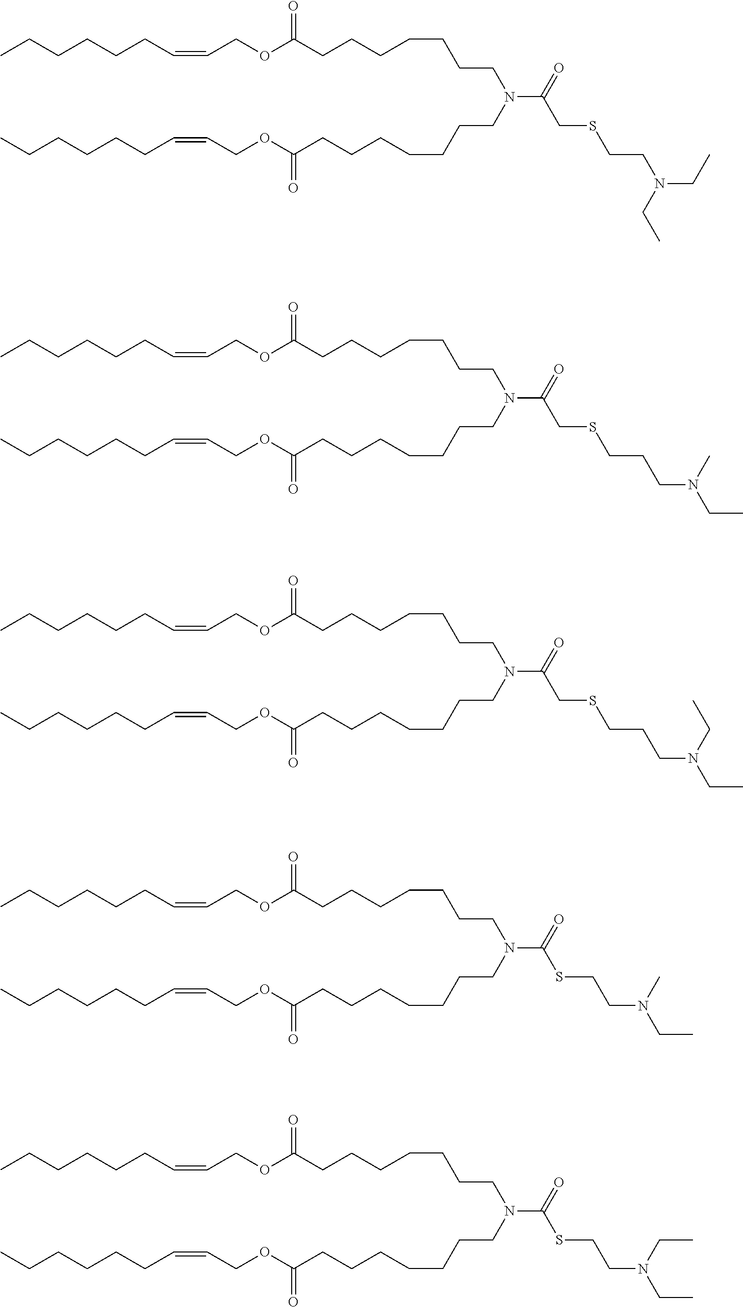

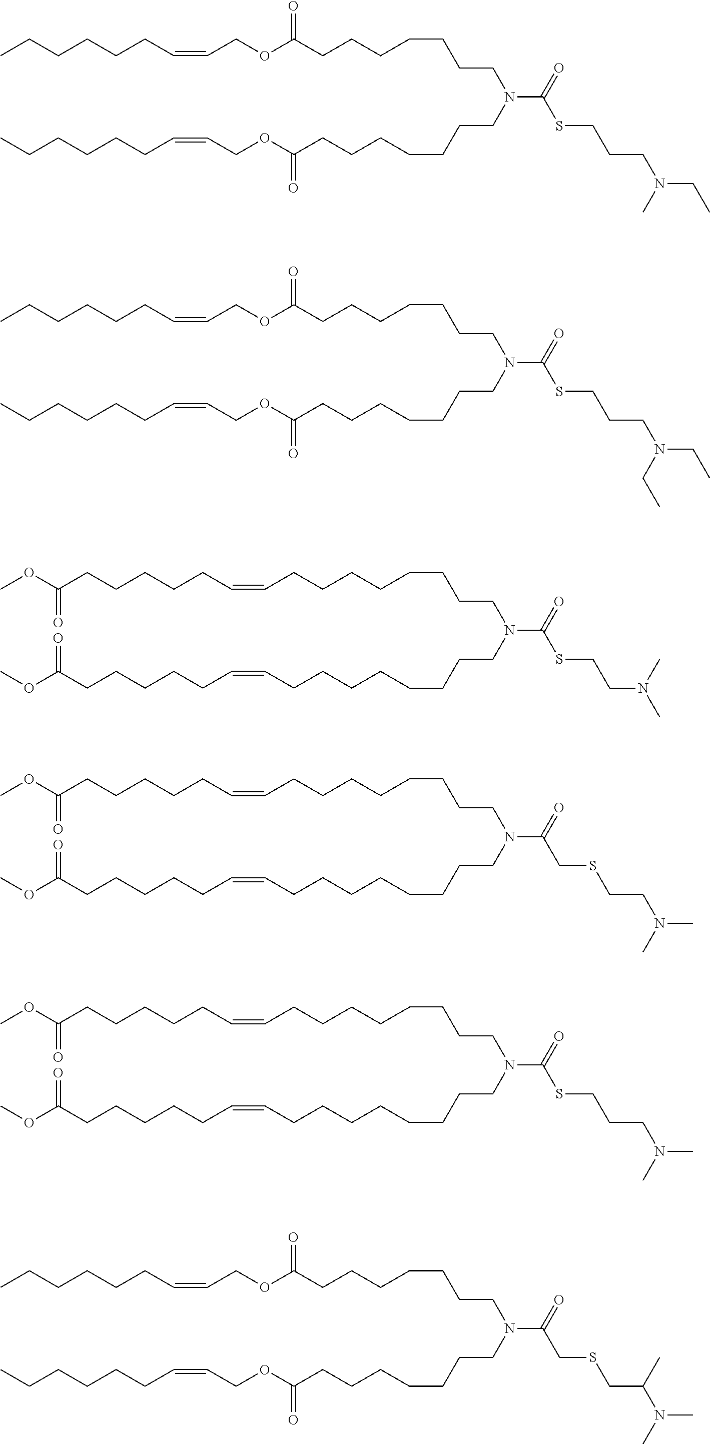

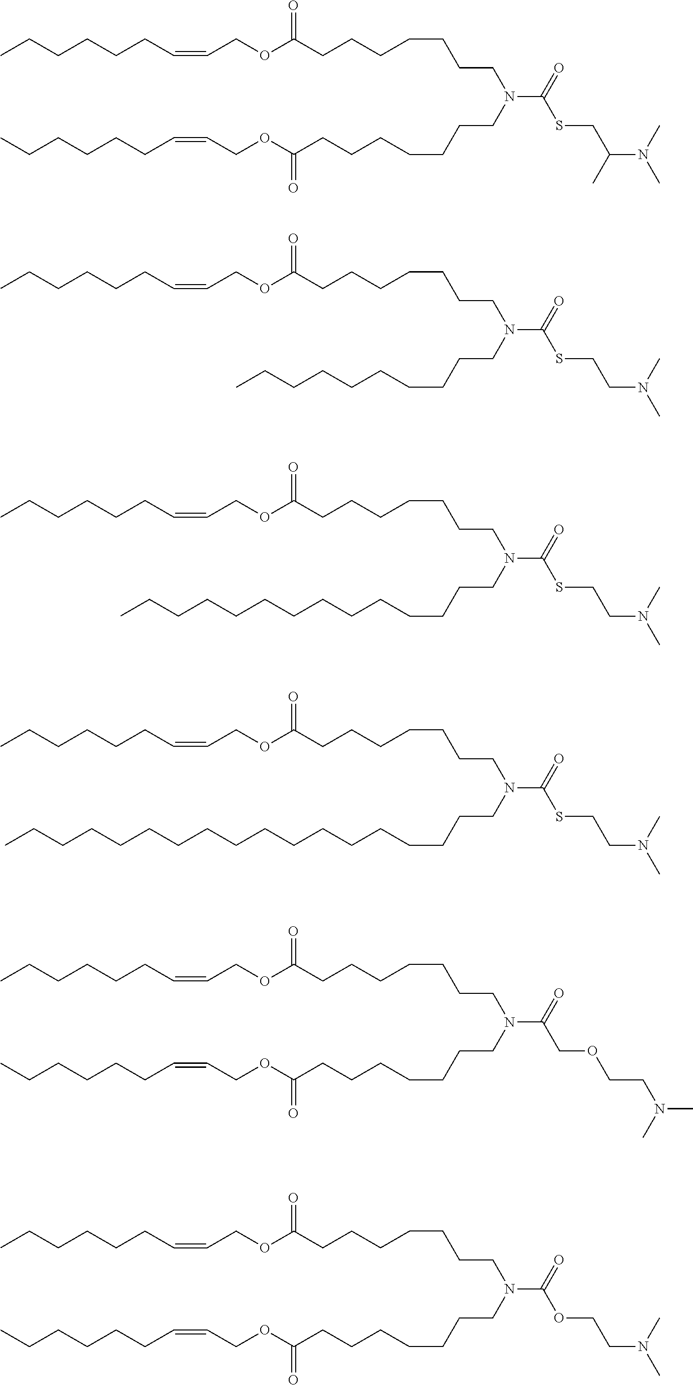

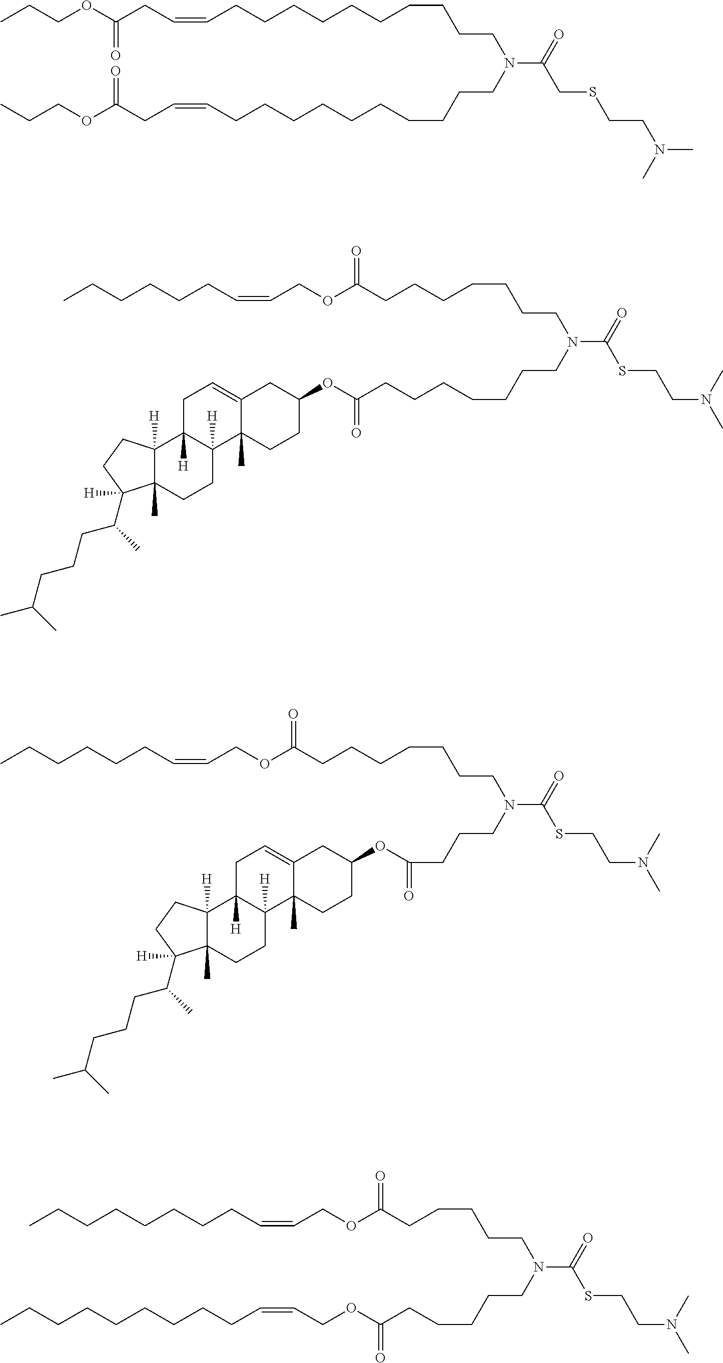

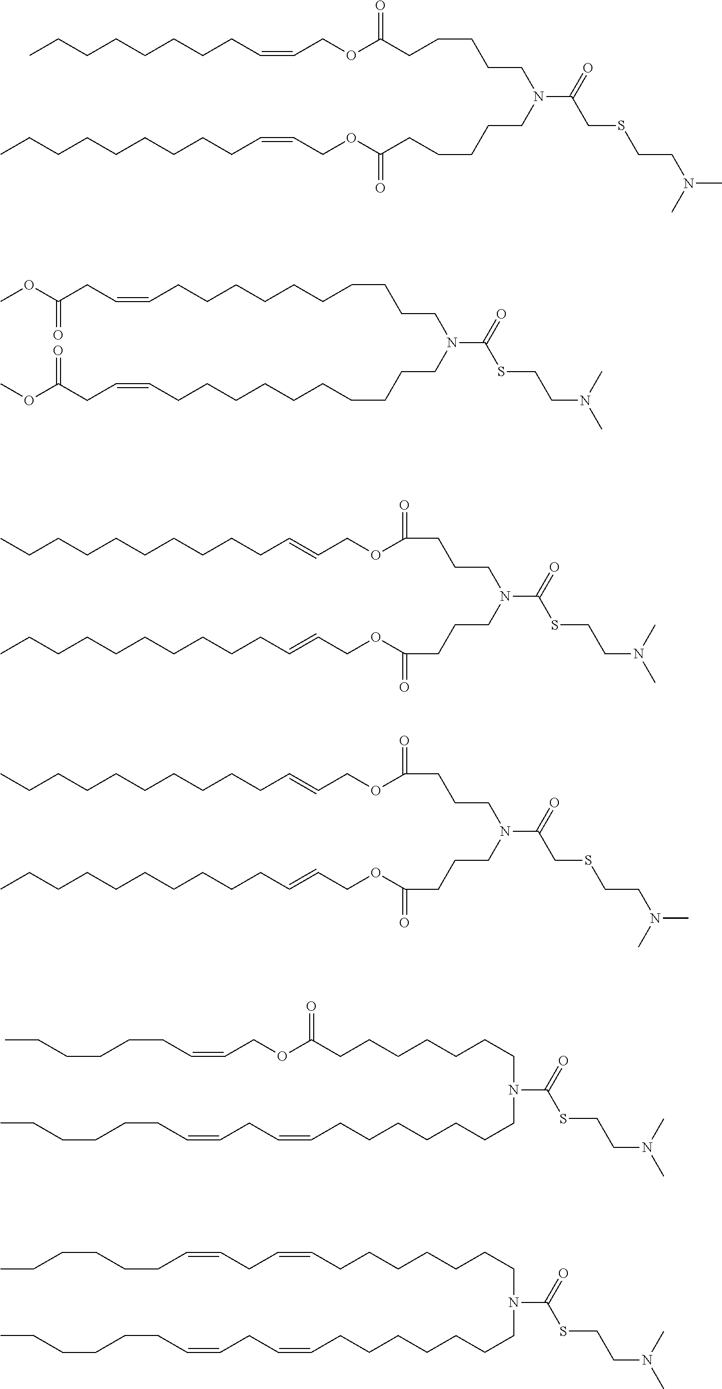

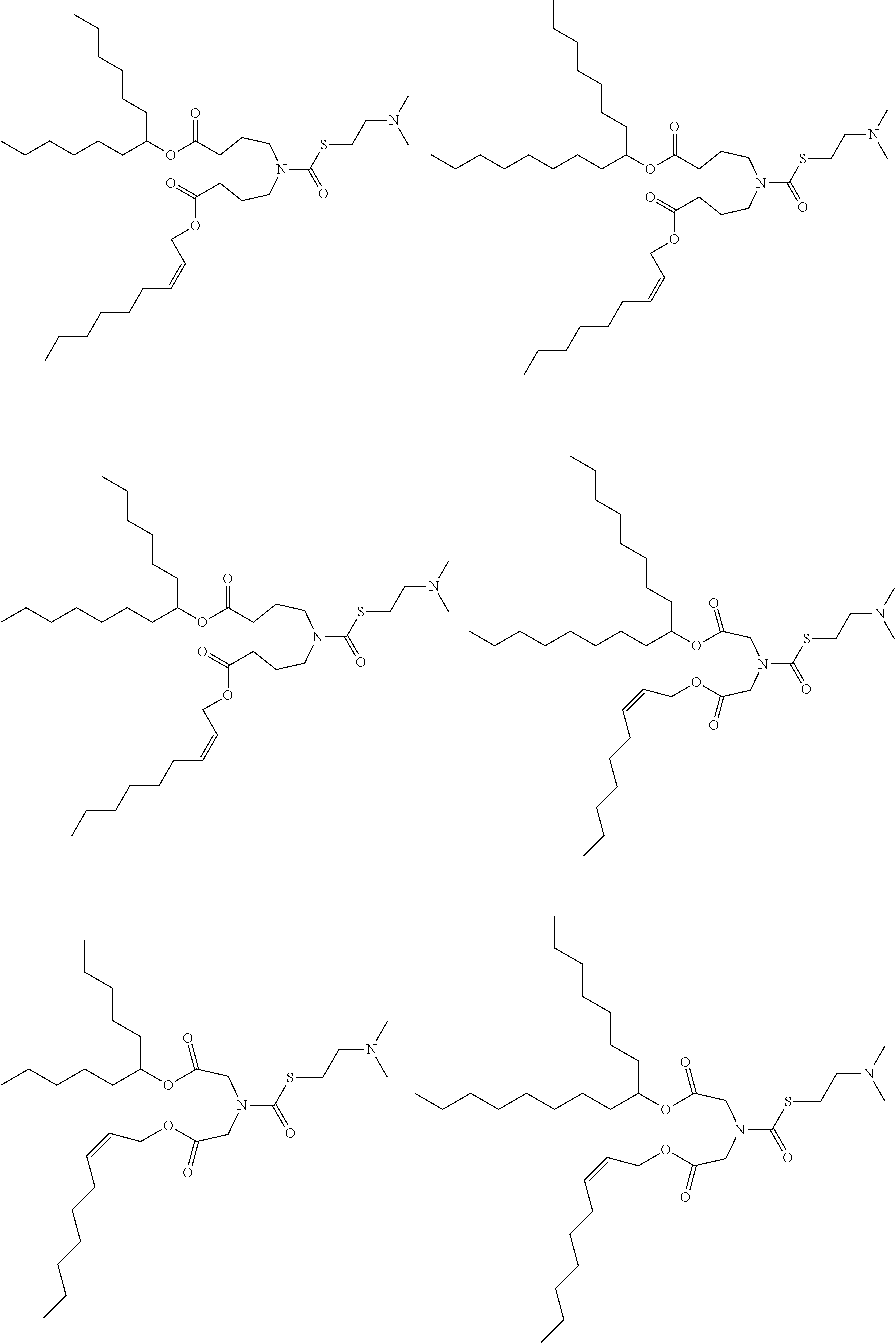

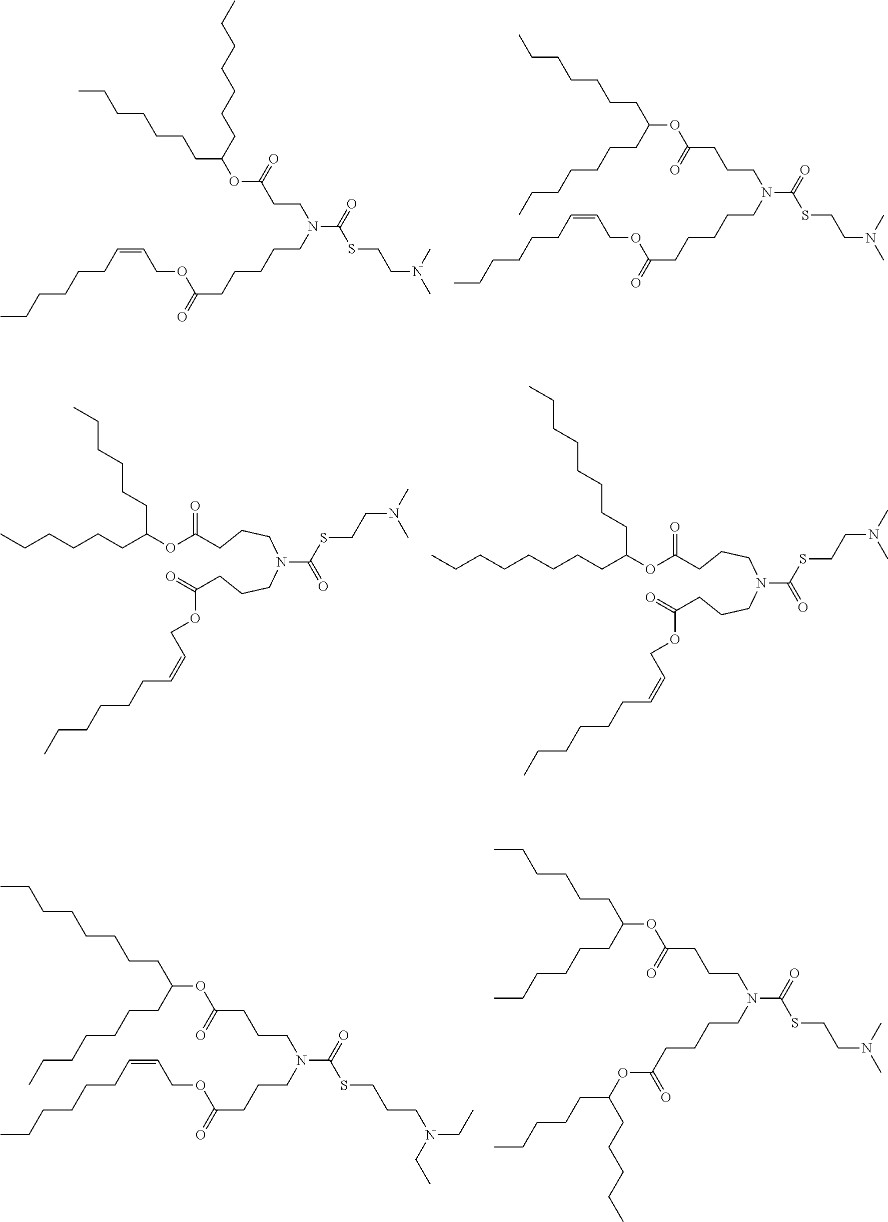

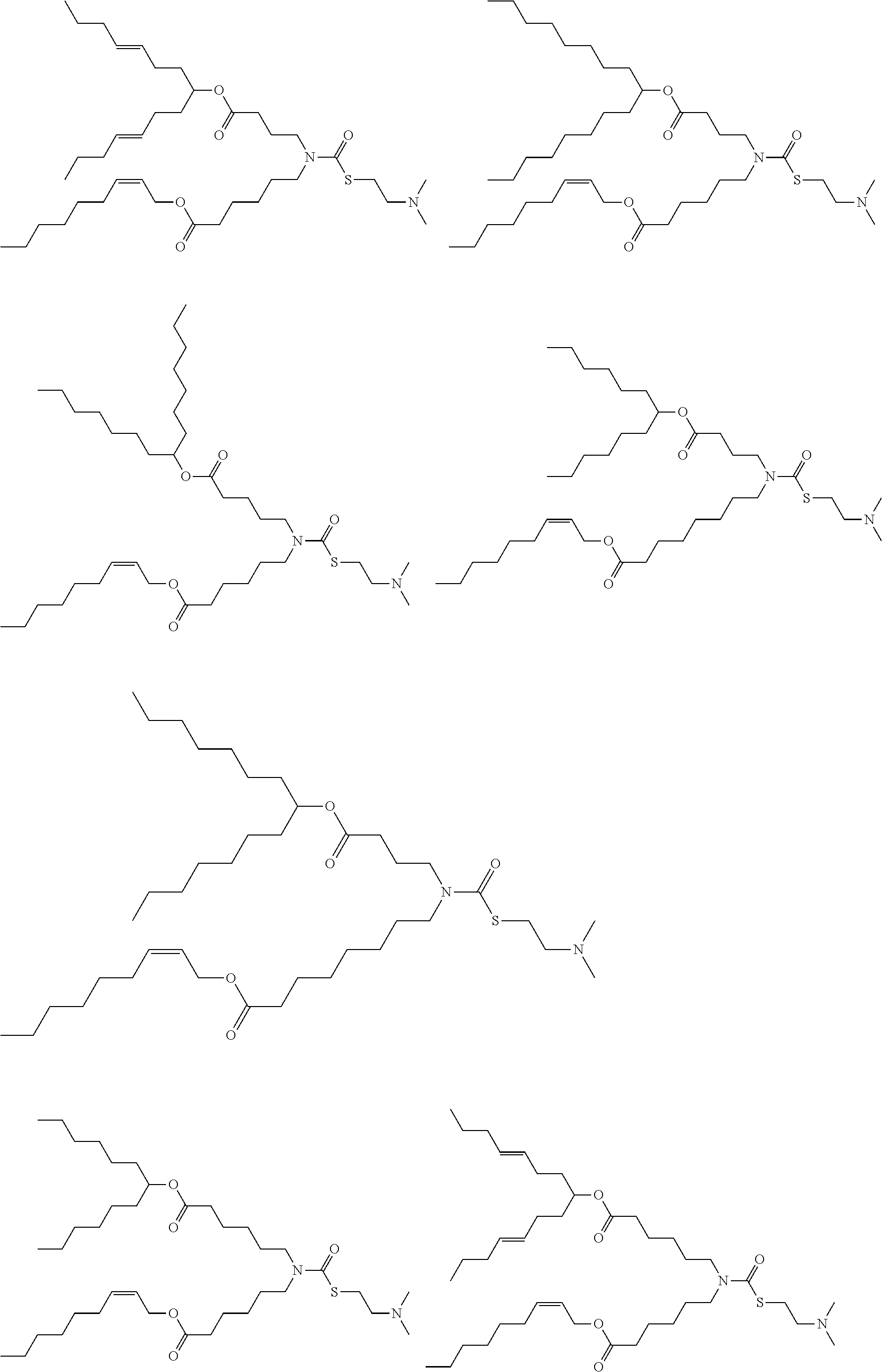

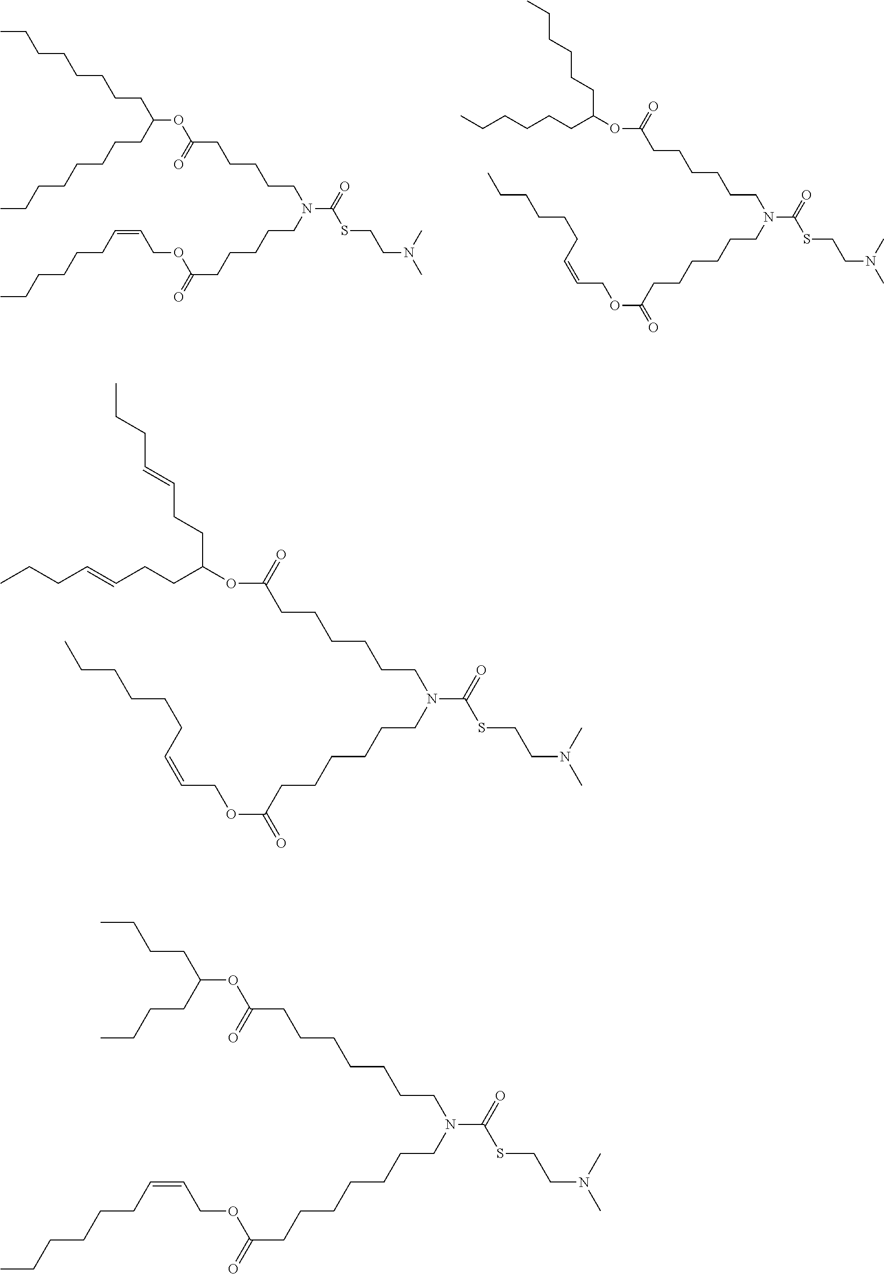

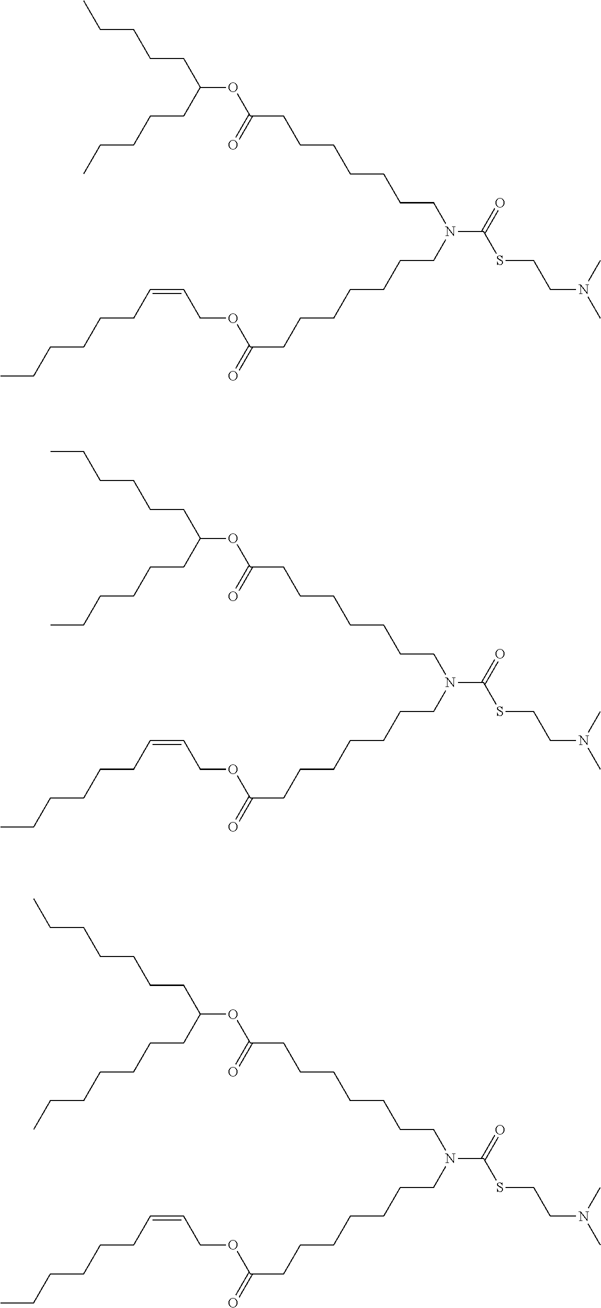

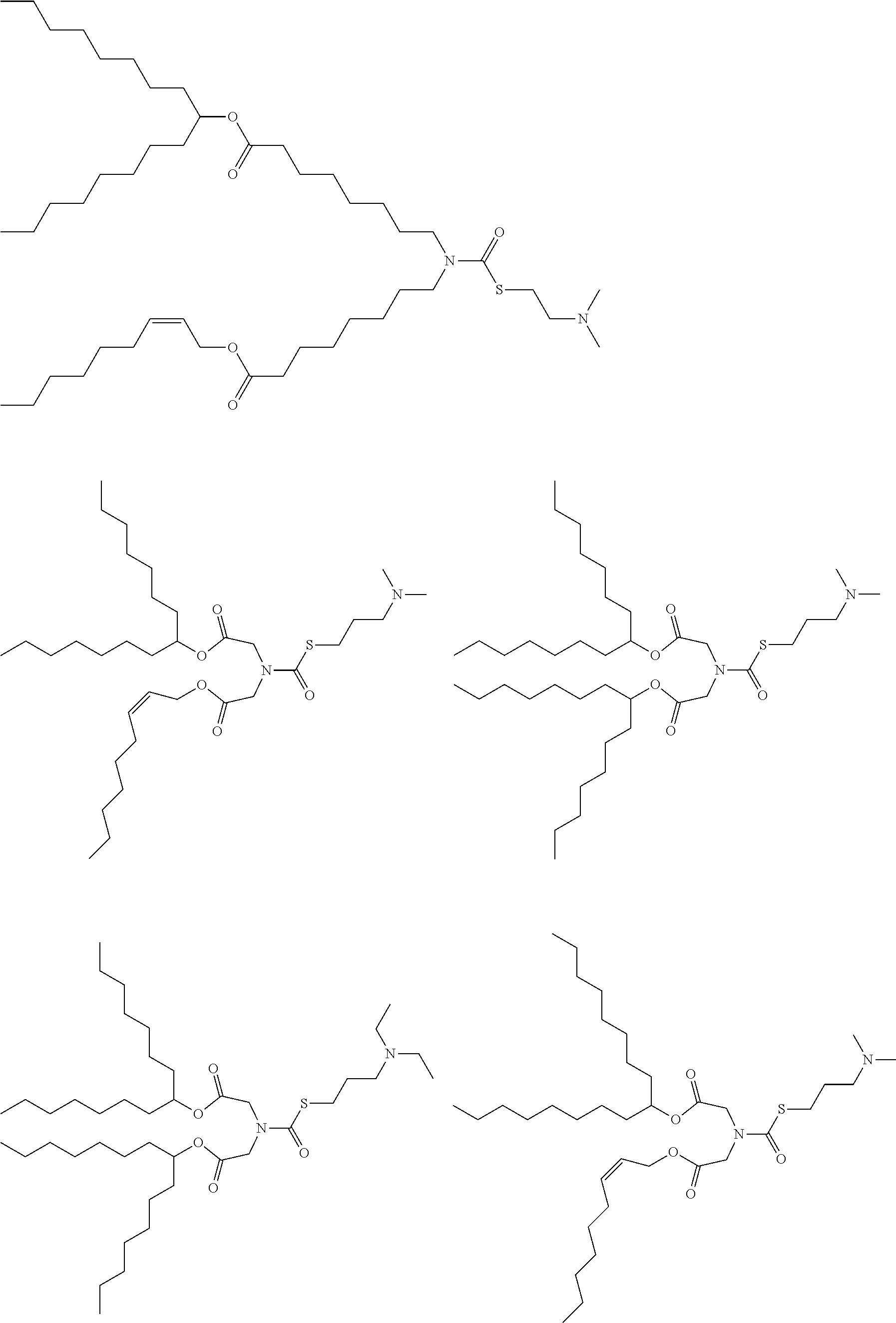

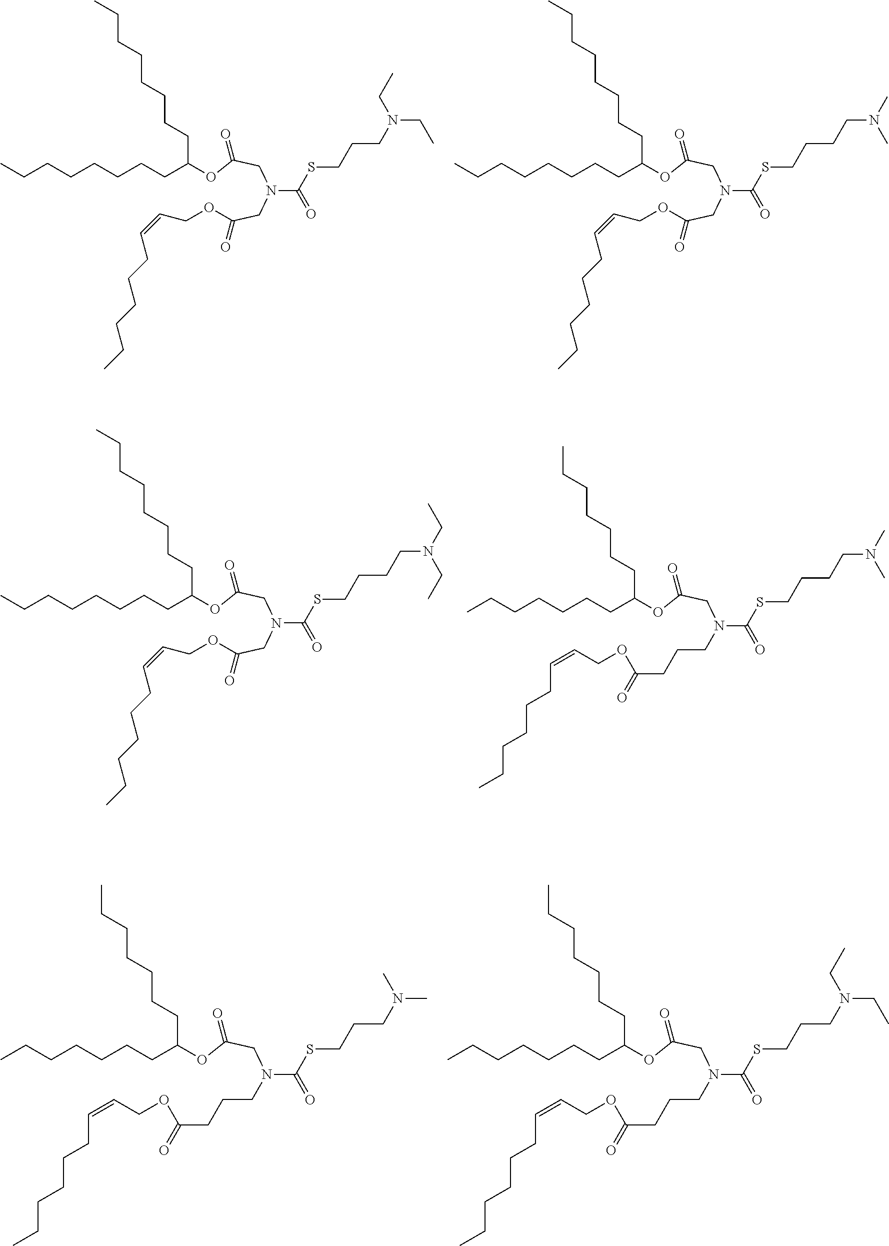

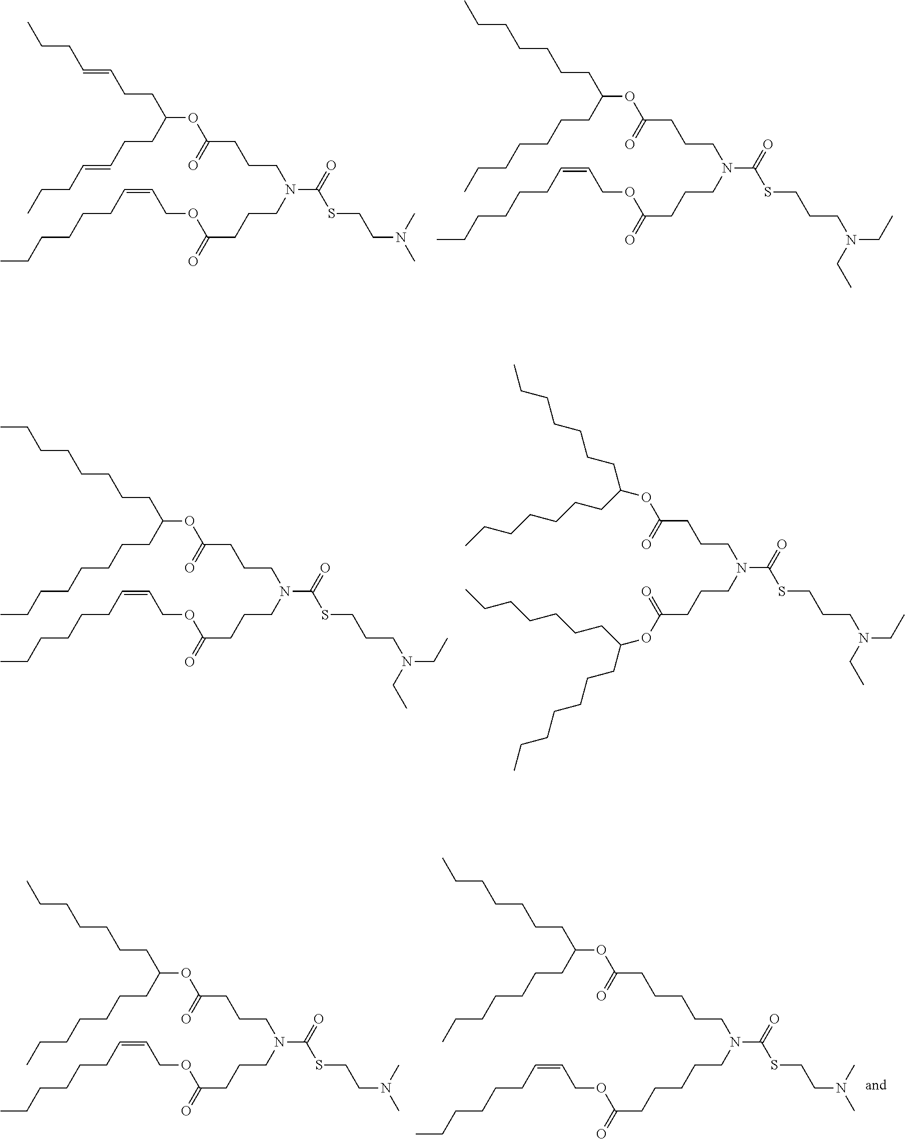

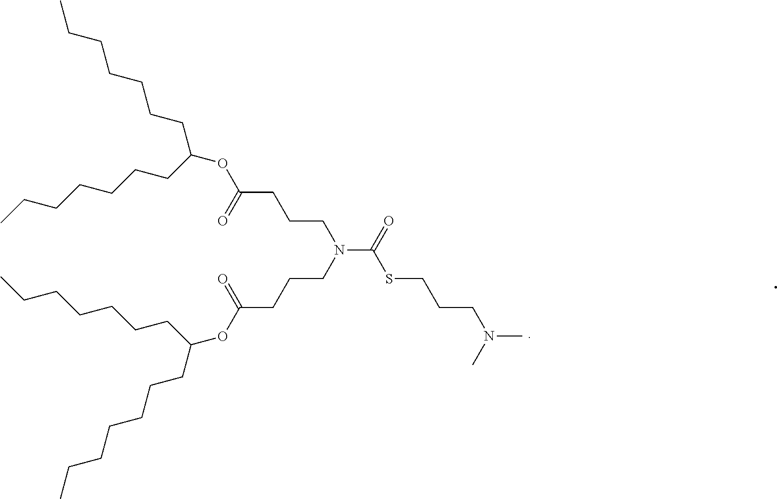 [SMILES: CCCCCCCC(CCCCCCC)OC(=O)CCCN(CCCC(=O)OC(CCCCCCC)CCCCCCC)C(=O)SCCCN(C)C]

In some embodiments, any one or more lipids recited herein may be expressly excluded.

### Helper Lipids and Sterols

The RNA replicon-lipid formulations of the present disclosure can comprise a helper lipid, which can be referred to as a neutral lipid, a neutral helper lipid, non-cationic lipid, non-cationic helper lipid, anionic lipid, anionic helper lipid, or a zwitterionic lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g., a cholesterol-free lipid formulation. In yet other embodiments, the helper lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 30 mol % to about 60 mol %, from about 32 mol % to about 58 mol %, from about 34 mol % to about 56 mol %, about 35 mol % to about 54 mol %, from about 36 mol % to about 52 mol %, from about 37 mol % to about 51 mol %, from about 38 mol % to about 50 mol %, or about 39 mol %, about 50 mol %, about 41 mol %, about 42 mol %, about 43 mol %, about 44 mol %, about 45 mol %, about 46 mol %, about 47 mol %, about 48 mol %, or about 49 mol % (or any fraction thereof or the range therein) of the total lipid present in the lipid formulation.

In some embodiments, the total of helper lipid in the formulation comprises two or more helper lipids and the total amount of helper lipid comprises from about 30 mol % to about 60 mol %, from about 32 mol % to about 58 mol %, from about 34 mol % to about 56 mol %, about 35 mol % to about 54 mol %, from about 36 mol % to about 52 mol %, from about 37 mol % to about 51 mol %, from about 38 mol % to about 50 mol %, or about 39 mol %, about 50 mol %, about 41 mol %, about 42 mol %, about 43 mol %, about 44 mol %, about 45 mol %, about 46 mol %, about 47 mol %, about 48 mol %, or about 49 mol % (or any fraction thereof or the range therein) of the total lipid present in the lipid formulation. In some embodiments, the helper lipids are a combination of DSPC and DOTAP. In some embodiments, the helper lipids are a combination of DSPC and DOTMA.

The cholesterol or cholesterol derivative in the lipid formulation may comprise up to about 50 mol %, about 35 mol %, about 40 mol %, about 45 mol %, or about 50 mol % of the total lipid present in the lipid formulation. In some embodiments, the cholesterol or cholesterol derivative comprises about 15 mol % to about 45 mol %, about 20 mol % to about 45 mol %, about 30 mol % to about 45 mol %, or about 35 mol %, about 36 mol %, about 37 mol %, about 38 mol %, about 39 mol %, about 40 mol %, about 41 mol %, about 42 mol %, about 43 mol %, about 44 mol %, or about 45 mol % of the total lipid present in the lipid formulation.

The percentage of helper lipid present in the lipid formulation is a target amount, and the actual amount of helper lipid present in the formulation may vary, for example, by ±5 mol %.

A lipid formulation containing a cationic lipid compound or ionizable cationic lipid compound may be on a molar basis about 30-60% cationic lipid compound, about 35-50% cholesterol, about 5-20% helper lipid, and about 0.5-5% of a polyethylene glycol (PEG) lipid, wherein the percent is of the total lipid present in the formulation. In some embodiments, the composition is about 40-50% cationic lipid compound, about 35-45% cholesterol, about 5-15% helper lipid, and about 0.5-3% of a PEG-lipid, wherein the percent is of the total lipid present in the formulation.

### Lipid Conjugates

The lipid formulations described herein may further comprise a lipid conjugate. The conjugated lipid is useful for preventing the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front. Pharmacol. 2015 Dec. 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps protect nanoparticles from the immune system and their escape from RES uptake (Nanomedicine (Lond). 2011 June; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (J. Nanomaterials. 2011; 2011:12). It has been shown that increased PEGylation leads to a significant increase in the circulation half-life of lipid formulations (Annu. Rev. Biomed. Eng. 2011 Aug. 15; 13:507-30; J. Control Release. 2010 Aug. 3; 145(3):178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG monomers can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 to about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

In some embodiments, the PEG-lipid is PEG550-PE. In some embodiments, the PEG-lipid is PEG750-PE. In some embodiments, the PEG-lipid is PEG2000-DMG. In some preferred embodiments, the PEG-lipid is 2-[(polyethylene glycol)-2000]-N,N-ditetradecylacetamide (also known as ALC-0159).

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of an RNA replicon-lipid formulation targeting hepatocytes described herein, the mRNA-lipid formulation enters hepatocytes through receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell's own internal translation processes will then translate the RNA replicon or combination of nucleic acid molecules into the encoded protein (e.g., HBV antigen). The encoded protein can further undergo post-translational processing, including transportation to a targeted organelle or location within the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid, e.g. RNA replicon or combination of nucleic acid molecules. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) a simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

In certain embodiments, examples of lipids and lipid nanoparticles, pharmaceutical compositions comprising the lipids, methods of making the lipids or formulating pharmaceutical compositions comprising the lipids and nucleic acid molecules, and methods of using the pharmaceutical compositions for treating or preventing diseases are described in U.S. or International Patent Application Publications, such as US2017/0190661, US2006/0008910, US2015/0064242, US2005/0064595, WO2019/036030, US2019/0022247, WO2019/036028, WO2019/036008, WO2019/036000, US2016/0376224, US2017/0119904, WO2018/200943, WO2018/191657, WO2018/118102, US2018/0169268, WO2018/118102, WO2018/119163, US2014/0255472, and US2013/0195968, the relevant content of each of which is hereby incorporated by reference in its entirety.

### Methods of Prime/Boost Immunization

Embodiments of the application also contemplate administering an immunogenically effective amount of a pharmaceutical composition or immunogenic combination to a subject, and subsequently administering another dose of an immunogenically effective amount of a pharmaceutical composition or immunogenic combination to the same subject, in a so-called prime-boost regimen. Thus, in an embodiment, a pharmaceutical composition or immunogenic combination of the application is a primer vaccine used for priming an immune response. In another embodiment, a pharmaceutical composition or immunogenic combination of the application is a booster vaccine used for boosting an immune response. The priming and boosting vaccines of the application can be used in the methods of the application described herein. This general concept of a prime-boost regimen is well known to the skilled person in the vaccine field. Any of the pharmaceutical compositions and immunogenic combinations of the application described herein can be used as priming and/or boosting vaccines for priming and/or boosting an immune response against HBV. Preferably, methods for vaccinating a subject comprise administering to the subject a pharmaceutical composition comprising a nucleic acid molecule, nucleic acid combination, vector, or RNA replicon of the application, and administering to the subject a second composition comprising a nucleic acid molecule encoding at least one identical HBV antigen as a prime-boost regimen.

In some embodiments of the application, a pharmaceutical composition or immunogenic combination of the application can be administered for priming immunization. The pharmaceutical composition or immunogenic combination can be re-administered for boosting immunization. Further booster administrations of the pharmaceutical composition or vaccine combination can optionally be added to the regimen, as needed. An adjuvant can be present in a pharmaceutical composition of the application used for boosting immunization, present in a separate composition to be administered together with the pharmaceutical composition or immunogenic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a pharmaceutical composition or immunogenic combination of the application to a subject to prime the immune response; and subsequently administering another dose of an immunogenically effective amount of a pharmaceutical composition or immunogenic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the pharmaceutical composition or immunogenic combination, or other adjuvant, is administered.

The antigens in the priming and boosting compositions need not to be identical, but should share antigens or be substantially similar to each other. In certain embodiments, the vector of the boosting composition is different from the priming composition, e.g., an adenovirus vector, Modified Vaccinia Ankara (MVA) vector, DNA, or protein. The priming and boosting compositions of the invention can each comprise one, two, three or multiple doses.

### EMBODIMENTS

Embodiment 1 comprises a nucleic acid molecule or combination comprising a non-naturally occurring polynucleotide sequence comprising, ordered from the 5′- to 3′-end:
(1) a polynucleotide sequence encoding a first hepatitis B virus (HBV) antigen,
(2) a first internal ribosome entry sequence (IRES) element or a polynucleotide sequence encoding a first autoprotease peptide, and
a polynucleotide sequence encoding a second HBV antigen,

wherein at least one of the first and second HBV antigens is an HBV surface antigen.

Embodiment 1a comprises the nucleic acid molecule or combination of embodiment 1, wherein the first and second HBV antigens are independently selected from the group consisting of an HBV core antigen, an HBV polymerase (pol) antigen, and an HBV surface antigen.

Embodiment 1b comprises the nucleic acid molecule or combination of embodiment 1 or 1a, wherein at least one of the first and second HBV antigens is an HBV Pre-S1 antigen or an HBV PreS2.S antigen.

Embodiment 2 comprises the nucleic acid molecule or combination of any one of embodiments 1-1b, wherein one of the first or second HBV antigens is an HBV core antigen or HBV pol antigen.

Embodiment 3 comprises the nucleic acid molecule or combination of any one of embodiments 1-2, wherein the non-naturally occurring polynucleotide sequence further comprises, ordered from the 5′- to 3′-end:
(3) a second IRES element or a polynucleotide sequence encoding a second autoprotease peptide operably linked to the 3′ end of the polynucleotide sequence encoding the second HBV antigen, and
(4) a polynucleotide sequence encoding a third HBV antigen.

Embodiment 3a comprises the nucleic acid molecule or combination of embodiment 3, wherein the third HBV antigen is independently selected from the group consisting of an HBV core antigen, an HBV polymerase (pol) antigen, and an HBV surface antigen.

Embodiment 4 comprises the nucleic acid molecule or combination of embodiment 3 or 3a, wherein the non-naturally occurring polynucleotide sequence further comprises, ordered from the 5′- to 3′-end:
(5) a third IRES element or a polynucleotide sequence encoding a third autoprotease peptide operably linked to the 3′ end of the polynucleotide sequence encoding the third HBV antigen, and
(6) a polynucleotide sequence encoding a fourth HBV antigen.

Embodiment 4a comprises the nucleic acid molecule or combination of embodiment 4, wherein the third HBV antigen is independently selected from the group consisting of an HBV core antigen, an HBV polymerase (pol) antigen, and an HBV surface antigen.

Embodiment 4b comprises the nucleic acid molecule or combination of embodiment 1-2, comprising a first non-naturally occurring polynucleotide sequence comprising, ordered from the 5′- to 3′-end:
(1) a polynucleotide sequence encoding a first hepatitis B virus (HBV) antigen,
(2) a first internal ribosome entry sequence (IRES) element or a polynucleotide sequence encoding a first autoprotease peptide, and
(3) a polynucleotide sequence encoding a second HBV antigen, and

a second non-naturally occurring polynucleotide sequence comprising, ordered from the 5′- to 3′-end:
(1) a polynucleotide sequence encoding a third hepatitis B virus (HBV) antigen,
(2) a second internal ribosome entry sequence (IRES) element or a polynucleotide sequence encoding a second autoprotease peptide, and
(3) a polynucleotide sequence encoding a fourth HBV antigen,

wherein the first and second non-naturally occurring polynucleotide sequence are linked by a third internal ribosome entry sequence (IRES) element or a polynucleotide sequence encoding a third autoprotease peptide, or are present in separate nucleic acid molecules, and

wherein the first, second, third and fourth HBV antigens are each independently selected from the group consisting of an HBV core antigen, an HBV polymerase (pol) antigen, and an HBV surface antigen, and at least one of the first, second, third and fourth HBV antigens is an HBV surface antigen selected from an HBV Pre-S1 antigen having an amino acid sequence at least 98% identical to the amino acid sequence of SEQ ID NO: 1 or SEQ ID NO: 3 and an HBV PreS2.S antigen having an amino acid sequence at least 98% identical to the amino acid sequence of SEQ ID NO: 5, preferably one of the first, second, third or fourth HBV antigens is an HBV core or an HBV pol antigen.

Embodiment 5 comprises the nucleic acid molecule or combination of embodiment 1 to 4b, wherein each of the first, second, third and fourth HBV antigens is different from each other.

Embodiment 6 comprises the nucleic acid molecule or combination of any one of embodiments 1-5, wherein each of the first, second, third and fourth HBV antigens is independently selected from the group consisting of:
(i) a first HBV PreS1 antigen comprising, preferably consisting of, an amino acid sequence that is at least 98% identical to the amino acid sequence of SEQ ID NO: 1, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 1;
(ii) a second HBV PreS1 antigen comprising, preferably consisting of, an amino acid sequence that is at least 98% identical to the amino acid sequence of SEQ ID NO: 3, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 3;
(iii) an HBV PreS2 antigen comprising, preferably consisting of, an amino acid sequence that is at least 98% identical to the amino acid sequence of SEQ ID NO: 5, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 5;
(iv) an HBV core antigen comprising, preferably consisting of, an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7; and
(v) an HBV polymerase antigen comprising, preferably consisting of, an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9.

Embodiment 6a comprises the nucleic acid molecule or combination of embodiment 6, wherein the HBV core antigen comprises, preferably consists of, an amino acid sequence that is at least 90% identical to SEQ ID NO: 86, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 86.

Embodiment 6b comprises the nucleic acid molecule or combination of embodiment 6, wherein each of the first, second, third and fourth HBV antigens is independently selected from the group consisting of:
(1) the first HBV Pre-S1 antigen comprising the amino acid sequence of SEQ ID NO: 1;
(2) the second HBV Pre-S1 antigen comprising the amino acid sequence of SEQ ID NO: 3;
(3) the HBV PreS2.S antigen comprising the amino acid sequence of SEQ ID NO: 5;
(4) the HBV core antigen comprising the amino acid sequence of SEQ ID NO: 7; and
(5) the HBV polymerase antigen comprising the amino acid sequence of SEQ ID NO: 9.

Embodiment 6b1 comprises the nucleic acid molecule or combination of embodiment 6b, wherein each of the first, second, third and fourth HBV antigens is independently selected from the group consisting of:
(1) the first HBV Pre-S1 antigen consisting of the amino acid sequence of SEQ ID NO: 1;
(2) the second HBV Pre-S1 antigen consisting of the amino acid sequence of SEQ ID NO: 3;
(3) the HBV PreS2.S antigen consisting of the amino acid sequence of SEQ ID NO: 5;
(4) the HBV core antigen consisting of the amino acid sequence of SEQ ID NO: 86; and
(5) the HBV polymerase antigen consisting of the amino acid sequence of SEQ ID NO: 9.

Embodiment 6b2 comprises the nucleic acid molecule or combination of embodiment 6b, wherein each of the first, second, third and fourth HBV antigens is independently selected from the group consisting of:
(1) the first HBV Pre-S1 antigen consisting of the amino acid sequence of SEQ ID NO: 1;
(2) the second HBV Pre-S1 antigen consisting of the amino acid sequence of SEQ ID NO: 3;
(3) the HBV PreS2.S antigen consisting of the amino acid sequence of SEQ ID NO: 5;
(4) the HBV core antigen consisting of the amino acid sequence of SEQ ID NO: 84; and
(5) the HBV polymerase antigen consisting of the amino acid sequence of SEQ ID NO: 9.

Embodiment 6b3 comprises the nucleic acid molecule or combination of embodiment 6b, wherein each of the first, second, third and fourth HBV antigens is independently selected from the group consisting of:
(1) the first HBV Pre-S1 antigen consisting of the amino acid sequence of SEQ ID NO: 1;
(2) the second HBV Pre-S1 antigen consisting of the amino acid sequence of SEQ ID NO: 3;
(3) the HBV PreS2.S antigen consisting of the amino acid sequence of SEQ ID NO: 5;
(4) the HBV core antigen consisting of the amino acid sequence of SEQ ID NO: 85; and
(5) the HBV polymerase antigen consisting of the amino acid sequence of SEQ ID NO: 9.

Embodiment 6b4 comprises the nucleic acid molecule or combination of embodiment 6b, wherein each of the first, second, third and fourth HBV antigens is independently selected from the group consisting of:
(1) the first HBV Pre-S1 antigen consisting of the amino acid sequence of SEQ ID NO: 1;
(2) the second HBV Pre-S1 antigen consisting of the amino acid sequence of SEQ ID NO: 3;
(3) the HBV PreS2.S antigen consisting of the amino acid sequence of SEQ ID NO: 5;
(4) the HBV core antigen consisting of the amino acid sequence of SEQ ID NO: 7; and
(5) the HBV polymerase antigen consisting of the amino acid sequence of SEQ ID NO: 9.

Embodiment 6c comprises the nucleic acid molecule or combination of any one of embodiments 1 to 6b4, wherein the nucleic acid molecule comprises a polynucleotide sequence encoding at least one of the first HBV Pre-S1 antigen, the second HBV Pre-S1 antigen and the HBV PreS2.S antigen, and a polynucleotide sequence encoding at least one of the HBV core antigen and the HBV polymerase antigen.

Embodiment 6c1 comprises the nucleic acid molecule or combination of any one of embodiments 1-6c, wherein each of the first and second HBV Pre-S1 antigens, the HBV core antigen and the HBV pol antigen is independently operably linked to a signal peptide, and the HBV PreS2.S antigen comprises an internal signal peptide.

Embodiment 6c2 comprises the nucleic acid molecule or combination of embodiment 6c1, wherein the signal peptide is a Cystatin S signal peptide, an Ig heavy chain gamma signal peptide SPIgG, an Ig heavy chain epsilon signal peptide SPIgE, or a short leader peptide sequence of the coronavirus.

Embodiment 6c3 comprises the nucleic acid molecule or combination of embodiment 6c2, wherein the signal peptide comprises the amino acid sequence of SEQ ID NO: 77 and is operably linked to the N-terminus of the HBV Pre-S1 antigens, the HBV core antigen and the HBV pol antigen.

Embodiment 6d comprises the nucleic acid molecule or combination of any one of embodiments 1 to 6c3, wherein the nucleic acid molecule comprises at least one IRES element.

Embodiment 6d1 comprises the nucleic acid molecule or combination of embodiment 6d, wherein the IRES element comprises the polynucleotide sequence of SEQ ID NO: 13.

Embodiment 6d2 comprises the nucleic acid molecule or combination of embodiment 6d1, wherein the IRES element consists of the polynucleotide sequence of SEQ ID NO: 13.

Embodiment 6d3 comprises the nucleic acid molecule or combination of embodiment 6d, wherein the IRES element comprises the polynucleotide sequence of SEQ ID NO: 14.

Embodiment 6d4 comprises the nucleic acid molecule or combination of embodiment 6d3, wherein the IRES element consists of the polynucleotide sequence of SEQ ID NO: 14.

Embodiment 6e comprises the nucleic acid molecule or combination of any one of embodiments 1 to 6d4, wherein the nucleic acid molecule comprises at least one polynucleotide sequence encoding an autoprotease peptide.

Embodiment 6e1 comprises the nucleic acid molecule or combination of embodiment 6e, wherein the autoprotease peptide comprises the amino acid sequence of SEQ ID NO: 11.

Embodiment 6e2 comprises the nucleic acid molecule or combination of embodiment 6e1, wherein the autoprotease peptide consists of the amino acid sequence of SEQ ID NO: 11.

Embodiment 6e3 comprises the nucleic acid molecule or combination of embodiment 6e, wherein the autoprotease peptide is encoded by a polynucleotide sequence comprises the sequence of SEQ ID NO: 12.

Embodiment 6e4 comprises the nucleic acid molecule or combination of embodiment 6e, wherein the autoprotease peptide is encoded by a polynucleotide sequence consists of the sequence of SEQ ID NO: 12.

Embodiment 7 comprises the nucleic acid molecule or combination of any one of embodiments 6c-6e4, wherein the HBV core antigen comprises, preferably consists of, an amino acid sequence that is at least 98% identical to at least one of SEQ ID NOs: 84, 85 or 86, such as at least 98%, 98.5%, 99%, 99.1%, 99.2%, 99.3%, 99.4%, 99.5%, 99.6%, 99.7%, 99.8%, 99.9% or 100% identical to SEQ ID NOs: 84, 85 or 86.

Embodiment 7a comprises the nucleic acid molecule or combination of embodiment 7, wherein the HBV core antigen comprises, preferably consists of, an amino acid sequence selected from the group consisting of SEQ ID NO: 84, SEQ ID NO: 85, or SEQ ID NO: 86.

Embodiment 7b comprises the nucleic acid molecule or combination of embodiment 7a, wherein the HBV core antigen consists of the amino acid sequence of SEQ ID NO: 86.

Embodiment 8 comprises the nucleic acid molecule or combination of any one of embodiments 1-7b, wherein the last five C-terminal amino acids of the HBV core antigen comprise a VVR amino acid sequence, more particularly a VVRR (SEQ ID NO: 91) amino acid sequence, more particularly a VVRRR (SEQ ID NO: 92) amino acid sequence.

Embodiment 9 comprises the nucleic acid molecule or combination of any one of embodiments 1-8, wherein at least one of the HBV surface antigen, the HBV core antigen and the HBV polymerase antigen comprises:
(i) a consensus sequence for two or more, preferably all, of HBV genotypes A, B, C and D; and/or
(ii) one or more epitopes for HLA-A*11:01, HLA-A*24:02, HLA-A*02:01, HLA-A*A2402, HLA-A*A0101, or HLA-B*40:01.

Embodiment 9a comprises the nucleic acid molecule or combination of embodiment 9, wherein at least one of the HBV surface antigen, the HBV core antigen and the HBV polymerase antigen comprises one or more epitopes selected from the group consisting of HLA-A*11:01 epitopes, HLA-A*24:02 epitopes, HLA-A*02:01 epitopes, and HLA-A*A2402 epitopes.

Embodiment 9a1 comprises the nucleic acid molecule or combination of embodiment 9 or 9a, wherein at least one of the HBV surface antigen, the HBV core antigen and the HBV polymerase antigen comprises one or more epitopes selected from the group consisting of HLA-A*11:01 epitopes, HLA-A*24:02 epitopes, and HLA-A*02:01 epitopes.

Embodiment 9a2 comprises the nucleic acid molecule or combination of any one of embodiments 9-9a1, wherein at least one of the HBV surface antigen, the HBV core antigen and the HBV polymerase antigen comprises one or more epitopes for HLA-A*11:01.

Embodiment 9a3 comprises the nucleic acid molecule or combination of any one of embodiments 9-9a2, wherein each of the HBV surface antigen, the HBV core antigen and the HBV polymerase antigen comprises one or more epitopes for HLA-A*11:01.

Embodiment 9a4 comprises the nucleic acid molecule or combination of any one of embodiments 9-9a3, wherein each of the HBV preS1, the HBV preS2.S, the HBV core antigen and the HBV polymerase antigen comprises one or more epitopes for HLA-A*11:01.

Embodiment 9b comprises the nucleic acid molecule or combination of any one of embodiments 9-9a4, wherein each of the HBV surface antigen, the HBV core antigen and the HBV polymerase antigen comprises a consensus sequence for HBV genotypes A, B, C and D.

Embodiment 9c comprises the nucleic acid molecule or combination of embodiment 9, wherein at least one of the HBV polymerase antigen, the HBV pre-S1 antigen, and the HBV preS2.S antigen comprises one or more HLA-A*24:02 epitopes.

Embodiment 9c1 comprises the nucleic acid molecule or combination of embodiment 9 or 9c, wherein each of the HBV polymerase antigen, the HBV pre-S1 antigen, and the HBV preS2.S antigen comprises one or more HLA-A*24:02 epitopes.

Embodiment 9c2 comprises the nucleic acid molecule or combination of any one of embodiments 9-9c1, wherein the HBV preS2.S antigen comprises one or more HLA-A*24:02 epitopes.

Embodiment 9d comprises the nucleic acid molecule or combination of embodiment 9, wherein at least one of the HBV polymerase antigen and the HBV core antigen comprises one or more HLA-A*02:01 epitopes.

Embodiment 9d1 comprises the nucleic acid molecule or combination of embodiment 9d, wherein each of the HBV polymerase antigen and the HBV core antigen comprises one or more HLA-A*02:01 epitopes.

Embodiment 9e comprises the nucleic acid molecule or combination of embodiment 9, wherein the HBV preS2.S antigen comprises one or more HLA-A*A2402 epitopes.

Embodiment 9f comprises the nucleic acid molecule or combination of embodiment 9, wherein at least one of the HBV polymerase antigen and the HBV core antigen comprises one or more HLA-A*A0101 epitopes.

Embodiment 9f1 comprises the nucleic acid molecule or combination of embodiment 9f, wherein each of the HBV polymerase antigen and the HBV core antigen comprises one or more HLA-A*A0101 epitopes.

Embodiment 9g comprises the nucleic acid molecule or combination of embodiment 9, wherein the HBV core antigen comprises one or more HLA-B*40:01 epitopes.

Embodiment 9h comprises the nucleic acid molecule or combination of embodiment 9, wherein the HBV core antigen comprises one or more epitopes selected from the group consisting of HLA-A*11:01 epitopes, HLA-A*02:01 epitopes, HLA-A*A0101 epitopes, and HLA-B*40:01 epitopes.

Embodiment 9i comprises the nucleic acid molecule or combination of embodiment 9, wherein the HBV polymerase antigen comprises one or more epitopes selected from the group consisting of HLA-A*11:01 epitopes, HLA-A*24:02 epitopes, HLA-A*02:01 epitopes, and HLA-A*A0101 epitopes.

Embodiment 9j comprises the nucleic acid molecule or combination of embodiment 9, wherein the HBV pre-S1 antigen comprises one or more epitopes selected from the group consisting of HLA-A*11:01 epitopes and HLA-A*24:02 epitopes.

Embodiment 9k comprises the nucleic acid molecule or combination of embodiment 9, wherein the HBV preS2.S antigen comprises one or more epitopes selected from the group consisting of HLA-A*11:01 epitopes, HLA-A*24:02 epitopes and HLA-A*A2402 epitopes.

Embodiment 91 comprises the nucleic acid molecule or combination of embodiment 9, wherein each of the HBV surface antigen, the HBV core antigen and the HBV polymerase antigen comprises:
(i) a consensus sequence for HBV genotypes A, B, C and D; and
(ii) one or more epitopes for HLA-A*11:01, HLA-A*24:02, HLA-A*02:01, HLA-A*A0201, HLA-A*A2402 and FILA-A*A0101.

Embodiment 10 comprises the nucleic acid molecule or combination of any one of embodiments 1-91, wherein each of the polynucleotide sequences encoding the first, second, third and fourth HBV antigens is independently selected from the group consisting of:
(i) a polynucleotide sequence encoding the first HBV Pre-S1 antigen having a sequence that is at least 90% identical to SEQ ID NO: 2, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2;
(ii) a polynucleotide sequence encoding the second HBV Pre-S1 antigen having a sequence that is at least 90% identical to SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4;
(iii) a polynucleotide sequence encoding the HBV PreS2.S antigen having a sequence that is at least 90% identical to SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 6;
(iv) a polynucleotide sequence encoding the HBV core antigen having a sequence that is at least 90% identical to SEQ ID NO: 8, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 8; and
(v) the polynucleotide sequence encoding the HBV polymerase antigen having a sequence that is at least 90% identical to SEQ ID NO: 10, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 10,
   preferably, the polynucleotide sequence encoding each of the first and second HBV Pre-S1 antigens, the HBV core antigen and the HBV pol antigen is independently operably linked to a polynucleotide sequence encoding a signal peptide, and the HBV PreS2.S antigen comprises an internal signal peptide.

Embodiment 10a comprises the nucleic acid molecule or combination of embodiment 10, wherein each of the polynucleotide sequences encoding the first, second, third and fourth HBV antigens is independently selected from the group consisting of:
(i) a polynucleotide sequence encoding the first HBV Pre-S1 antigen having the nucleotide sequence of SEQ ID NO: 2;
(ii) a polynucleotide sequence encoding the second HBV Pre-S1 antigen having the nucleotide sequence of SEQ ID NO: 4;
(iii) a polynucleotide sequence encoding the HBV PreS2.S antigen having the nucleotide sequence of SEQ ID NO: 6;
(iv) a polynucleotide sequence encoding the HBV core antigen having the nucleotide sequence of SEQ ID NO: 89; and
(v) the polynucleotide sequence encoding the HBV polymerase antigen having the nucleotide sequence of SEQ ID NO: 10.

Embodiment 10b comprises the nucleic acid molecule or combination of embodiment 10, wherein each of the polynucleotide sequences encoding the first, second, third and fourth HBV antigens is independently selected from the group consisting of:
(i) a polynucleotide sequence encoding the first HBV Pre-S1 antigen consisting of the nucleotide sequence of SEQ ID NO: 2;
(ii) a polynucleotide sequence encoding the second HBV Pre-S1 antigen consisting of the nucleotide sequence of SEQ ID NO: 4;
(iii) a polynucleotide sequence encoding the HBV PreS2.S antigen consisting of the nucleotide sequence of SEQ ID NO: 6;
(iv) a polynucleotide sequence encoding the HBV core antigen consisting of the nucleotide sequence of SEQ ID NO: 87; and
(v) the polynucleotide sequence encoding the HBV polymerase antigen consisting of the nucleotide sequence of SEQ ID NO: 10.

Embodiment 10c comprises the nucleic acid molecule or combination of embodiment 10, wherein each of the polynucleotide sequences encoding the first, second, third and fourth HBV antigens is independently selected from the group consisting of:
(i) a polynucleotide sequence encoding the first HBV Pre-S1 antigen consisting of the nucleotide sequence of SEQ ID NO: 2;
(ii) a polynucleotide sequence encoding the second HBV Pre-S1 antigen consisting of the nucleotide sequence of SEQ ID NO: 4;
(iii) a polynucleotide sequence encoding the HBV PreS2.S antigen consisting of the nucleotide sequence of SEQ ID NO: 6;
(iv) a polynucleotide sequence encoding the HBV core antigen consisting of the nucleotide sequence of SEQ ID NO: 88; and
(v) the polynucleotide sequence encoding the HBV polymerase antigen consisting of the nucleotide sequence of SEQ ID NO: 10.

Embodiment 10d comprises the nucleic acid molecule or combination of embodiment 10, wherein each of the polynucleotide sequences encoding the first, second, third and fourth HBV antigens is independently selected from the group consisting of:
(i) a polynucleotide sequence encoding the first HBV Pre-S1 antigen consisting of the nucleotide sequence of SEQ ID NO: 2;
(ii) a polynucleotide sequence encoding the second HBV Pre-S1 antigen consisting of the nucleotide sequence of SEQ ID NO: 4;
(iii) a polynucleotide sequence encoding the HBV PreS2.S antigen consisting of the nucleotide sequence of SEQ ID NO: 6;
(iv) a polynucleotide sequence encoding the HBV core antigen consisting of the nucleotide sequence of SEQ ID NO: 89; and
(v) the polynucleotide sequence encoding the HBV polymerase antigen consisting of the nucleotide sequence of SEQ ID NO: 10.

Embodiment 11 comprises the nucleic acid molecule or combination of embodiment 10, wherein the polynucleotide sequence encoding the HBV core antigen comprises, preferably consists of, a polynucleotide sequence that is at least 90% identical to SEQ ID NO: 87, SEQ ID NO: 88 or SEQ ID NO: 89, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 87, SEQ ID NO: 88 or SEQ ID NO: 89.

Embodiment 11a comprises the nucleic acid molecule or combination of embodiment 11, wherein the polynucleotide sequence encoding the HBV core antigen comprises, preferably consists of, any one of SEQ ID NO: 87, SEQ ID NO: 88 or SEQ ID NO: 89.

Embodiment 11b comprises the nucleic acid molecule or combination of embodiment 11 or 11a, wherein the polynucleotide sequence encoding the HBV core antigen consists of SEQ ID NO: 89.

Embodiment 11c comprises the nucleic acid molecule or combination of any one of embodiments 10 to 11b, wherein the polynucleotide sequence encoding each of the first and second HBV Pre-S1 antigens, the HBV core antigen and the HBV pol antigen is independently operably linked to a polynucleotide encoding a signal peptide, such as a Cystatin S signal peptide, an Ig heavy chain gamma signal peptide SPIgG, an Ig heavy chain epsilon signal peptide SPIgE, or a short leader peptide sequence of the coronavirus.

Embodiment 11d comprises the nucleic acid molecule or combination of embodiment 11c, wherein the polynucleotide encoding the signal peptide comprises the nucleotide sequence of SEQ ID NO: 90.

Embodiment 12 comprises the nucleic acid molecule or combination of any one of embodiments 1-11d, wherein each of the first, second and third autoprotease peptides independently comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof.

Embodiment 12a comprises the nucleic acid molecule or combination of embodiment 12, wherein each of the first, second and third autoprotease peptides comprises the peptide sequence of P2A, such as a P2A sequence of SEQ ID NO: 11.

Embodiment 13 comprises the nucleic acid molecule or combination of any one of embodiments 1-12a, wherein each of the first, second and third IRES is derived from encephalomyocarditis virus (EMCV) or Enterovirus 71 (EV71).

Embodiment 13a comprises the nucleic acid molecule or combination of embodiment 13, wherein each of the first, second and third IRES comprises the polynucleotide sequence of SEQ ID NO: 13 or 14.

Embodiment 14 comprises the nucleic acid molecule or combination of any one of embodiments 1-3a, comprising a non-naturally occurring polynucleotide sequence, having, ordered from the 5′- to 3′-end:
(1) a polynucleotide sequence encoding an HBV core antigen having the amino acid sequence of SEQ ID NO: 7, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen having the amino acid sequence of SEQ ID NO: 9;
(2) a polynucleotide sequence encoding an HBV polymerase antigen having the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen having the amino acid sequence of SEQ ID NO: 7;
(3) a polynucleotide sequence encoding an HBV Pre-S1 antigen having the amino acid sequence of SEQ ID NO: 1 or 3, a polynucleotide sequence encoding a P2A amino acid sequence of SEQ ID NO: 11 or an IRES having the polynucleotide sequence of SEQ ID NO: 13 or 14, and a polynucleotide sequence encoding an HBV PreS2.S antigen having the amino acid sequence of SEQ ID NO: 5;
(4) a polynucleotide sequence encoding an HBV PreS2.S antigen having the amino acid sequence of SEQ ID NO: 5, a polynucleotide sequence encoding a P2A amino acid sequence of SEQ ID NO: 11 or an IRES having the polynucleotide sequence of SEQ ID NO: 13 or 14, and a polynucleotide sequence encoding an HBV Pre-S1 antigen having the amino acid sequence of SEQ ID NO: 1 or 3;
(5) a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(6) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(7) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV polymerase antigen having the amino acid sequence of SEQ ID NO: 9;
(8) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7;
(9) a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(10) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(11) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV polymerase antigen having the amino acid sequence of SEQ ID NO: 9;
(12) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7;
(13) a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(14) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(15) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV polymerase antigen having the amino acid sequence of SEQ ID NO: 9;
(16) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7;
(17) a polynucleotide sequence encoding an HBV PreS2.S antigen having the amino acid sequence of SEQ ID NO: 5, an IRES having the polynucleotide sequence of SEQ ID NO: 13 or 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7;
(18) a polynucleotide sequence encoding an HBV PreS2.S antigen having the amino acid sequence of SEQ ID NO: 5, an IRES having the polynucleotide sequence of SEQ ID NO: 14, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV polymerase antigen having the amino acid sequence of SEQ ID NO: 9;
(19) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, an IRES having the polynucleotide sequence of SEQ ID NO: 13 or 14, and a polynucleotide sequence encoding an HBV PreS2.S antigen having the amino acid sequence of SEQ ID NO: 5;
(20) a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13 or 14, and a polynucleotide sequence encoding an HBV PreS2.S antigen having the amino acid sequence of SEQ ID NO: 5;
(21) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of SEQ ID NO: 7;
(22) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV polymerase antigen having the amino acid sequence of SEQ ID NO: 9;
(23) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of SEQ ID NO: 7, a polynucleotide sequence encoding a P2A amino acid sequence of SEQ ID NO: 11, and a polynucleotide sequence encoding an HBV PreS2.S antigen having the amino acid sequence of SEQ ID NO: 5; and
(24) a polynucleotide sequence encoding an HBV core antigen having the amino acid sequence of SEQ ID NO: 7, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV PreS2.S antigen having the amino acid sequence of SEQ ID NO: 5.

Embodiment 14a1 comprises the nucleic acid molecule or combination of embodiment 14, comprising a non-naturally occurring polynucleotide sequence, having, ordered from the 5′- to 3′-end:
(1) a polynucleotide sequence encoding an HBV core consisting of the amino acid sequence of any one of SEQ ID NOs: 7, 84, 85, or 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen consisting of the amino acid sequence of SEQ ID NO: 9;
(2) a polynucleotide sequence encoding an HBV polymerase antigen consisting of the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen consisting of the amino acid sequence of any one of SEQ ID NOs: 7, 84, 85, or 86;
(5) a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(6) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(7) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(8) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of any one of SEQ ID NOs: 7, 84, 85, or 86;
(9) a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 13, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3; (10) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, an IRES having the polynucleotide sequence of SEQ ID NO: 13, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(11) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 13, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(12) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 13, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of any one of SEQ ID NOs: 7, 84, 85, or 86;
(13) a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 14, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(14) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, an IRES having the polynucleotide sequence of SEQ ID NO: 14, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(15) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 14, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(16) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 14, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of any one of SEQ ID NOs: 7, 84, 85, or 86;
(17) a polynucleotide sequence encoding an HBV PreS2.S antigen consisting of the amino acid sequence of SEQ ID NO: 5, an IRES having the polynucleotide sequence of SEQ ID NO: 13 or 14, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of any one of SEQ ID NOs: 7, 84, 85, or 86;
(18) a polynucleotide sequence encoding an HBV PreS2.S antigen consisting of the amino acid sequence of SEQ ID NO: 5, an IRES having the polynucleotide sequence of SEQ ID NO: 14, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(19) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, an IRES having the polynucleotide sequence of SEQ ID NO: 13 or 14, and a polynucleotide sequence encoding an HBV PreS2.S antigen consisting of the amino acid sequence of SEQ ID NO: 5;
(20) a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 13 or 14, and a polynucleotide sequence encoding an HBV PreS2.S antigen consisting of the amino acid sequence of SEQ ID NO: 5;
(21) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of any one of SEQ ID NOs: 7, 84, 85, or 86;
(22) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(23) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, and a polynucleotide sequence encoding an HBV Pre S2.S antigen consisting of the amino acid sequence of SEQ ID NO: 5; and
(24) a polynucleotide sequence encoding an HBV core antigen consisting of the amino acid sequence of any one of SEQ ID NOs: 7, 84, 85, or 86, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV PreS2.S antigen consisting of the amino acid sequence of SEQ ID NO: 5.

Embodiment 14a2 comprises the nucleic acid molecule or combination of embodiment 14, comprising a non-naturally occurring polynucleotide sequence, having, ordered from the 5′- to 3′-end:
(1) a polynucleotide sequence encoding an HBV core antigen consisting of the amino acid sequence of SEQ ID NO: 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen consisting of the amino acid sequence of SEQ ID NO: 9;
(2) a polynucleotide sequence encoding an HBV polymerase antigen consisting of the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen consisting of the amino acid sequence of SEQ ID NO: 86;
(3) a polynucleotide sequence encoding an HBV Pre-S1 antigen consisting of the amino acid sequence of SEQ ID NO: 1 or 3, a polynucleotide sequence encoding a P2A amino acid sequence of SEQ ID NO: 11 or an IRES having the polynucleotide sequence of SEQ ID NO: 13 or 14, and a polynucleotide sequence encoding an HBV PreS2.S antigen consisting of the amino acid sequence of SEQ ID NO: 5;
(4) a polynucleotide sequence encoding an HBV PreS2.S antigen consisting of the amino acid sequence of SEQ ID NO: 5, a polynucleotide sequence encoding a P2A amino acid sequence of SEQ ID NO: 11 or an IRES having the polynucleotide sequence of SEQ ID NO: 13 or 14, and a polynucleotide sequence encoding an HBV Pre-S1 antigen consisting of the amino acid sequence of SEQ ID NO: 1 or 3;
(5) a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(6) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(7) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, a polynucleotide sequence encoding a P2A amino acid sequence of SEQ ID NO: 11, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(8) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of SEQ ID NO: 86;
(9) a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 13, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(10) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of SEQ ID NO: 86, an IRES having the polynucleotide sequence of SEQ ID NO: 13, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(11) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 13, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(12) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 13, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of SEQ ID NO: 86;
(13) a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 14, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(14) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of SEQ ID NO: 86, an IRES having the polynucleotide sequence of SEQ ID NO: 14, a polynucleotide sequence encoding an HBV PreS2.S antigen consisting of the amino acid sequence of SEQ ID NO: 5, a polynucleotide sequence encoding a P2A amino acid sequence of SEQ ID NO: 11, and a polynucleotide sequence encoding an HBV Pre-S1 antigen consisting of the amino acid sequence of SEQ ID NO: 1 or 3;
(15) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 14, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(16) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV Pre-S1 antigen consisting of the amino acid sequence of SEQ ID NO: 1 or 3, an IRES having the polynucleotide sequence of SEQ ID NO: 14, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of SEQ ID NO: 86;
(17) a polynucleotide sequence encoding an HBV PreS2.S antigen consisting of the amino acid sequence of SEQ ID NO: 5, an IRES having the polynucleotide sequence of SEQ ID NO: 13 or 14, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of SEQ ID NO: 86;
(18) a polynucleotide sequence encoding an HBV PreS2.S antigen consisting of the amino acid sequence of SEQ ID NO: 5, an IRES having the polynucleotide sequence of SEQ ID NO: 14, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(19) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of SEQ ID NO: 86, an IRES having the polynucleotide sequence of SEQ ID NO: 13 or 14, and a polynucleotide sequence encoding an HBV PreS2.S antigen consisting of the amino acid sequence of SEQ ID NO: 5;
(20) a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV polymerase antigen consisting of the amino acid sequence of SEQ ID NO: 9, an IRES having the polynucleotide sequence of SEQ ID NO: 13 or 14, and a polynucleotide sequence encoding an HBV PreS2.S antigen consisting of the amino acid sequence of SEQ ID NO: 5;
(21) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV core antigen consisting of the amino acid sequence of SEQ ID NO: 86;
(22) a polynucleotide sequence encoding an HBV PreS2.S antigen consisting of the amino acid sequence of SEQ ID NO: 5, a polynucleotide sequence encoding a P2A amino acid sequence of SEQ ID NO: 11, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV polymerase antigen consisting of the amino acid sequence of SEQ ID NO: 9;
(23) a polynucleotide sequence encoding an HBV polymerase antigen consisting of the amino acid sequence of SEQ ID NO: 9, a polynucleotide sequence encoding a P2A amino acid sequence of SEQ ID NO: 11, a polynucleotide sequence encoding an HBV core antigen consisting of the amino acid sequence of SEQ ID NO: 86, a polynucleotide sequence encoding a P2A amino acid sequence of SEQ ID NO: 11, and a polynucleotide sequence encoding an HBV PreS2.S antigen consisting of the amino acid sequence of SEQ ID NO: 5; and
(24) a polynucleotide sequence encoding an HBV core antigen consisting of the amino acid sequence of SEQ ID NO: 86, a polynucleotide sequence encoding a P2A amino acid sequence of SEQ ID NO: 11, a polynucleotide sequence encoding an HBV polymerase antigen consisting of the amino acid sequence of SEQ ID NO: 9, a polynucleotide sequence encoding a P2A amino acid sequence of SEQ ID NO: 11, and a polynucleotide sequence encoding an HBV PreS2.S antigen consisting of the amino acid sequence of SEQ ID NO: 5.

Embodiment 14a3 comprises the nucleic acid molecule or combination of embodiment 14, 14a1 or 14a2, wherein the polynucleotide sequence encoding each of the first and second HBV Pre-S1 antigens, the HBV core antigen and the HBV pol antigen is independently operably linked to a polynucleotide sequence encoding a signal peptide.

Embodiment 14a4 comprises the nucleic acid molecule or combination of embodiment 14a3, wherein the signal peptide a Cystatin S signal peptide, an Ig heavy chain gamma signal peptide SPIgG, an Ig heavy chain epsilon signal peptide SPIgE, or a short leader peptide sequence of the coronavirus.

Embodiment 14a5 comprises the nucleic acid molecule or combination of embodiment 14a4, wherein the signal peptide comprises the amino acid sequence of SEQ ID NO: 77.

Embodiment 14a6 comprises the nucleic acid molecule or combination of embodiment 14a5, wherein the polynucleotide sequence encoding the signal peptide comprises the polynucleotide sequence of SEQ ID NO: 90.

Embodiment 14b comprises the nucleic acid molecule or combination of any one of embodiments 14 to 14a6, comprising the non-naturally occurring polynucleotide sequence of any one of embodiments 14(3) to 14(24), 14a1(5) to 14a1(24) or 14a2(3) to 14a2(24).

Embodiment 14c comprises the nucleic acid molecule or combination of embodiment 14b, comprising the non-naturally occurring polynucleotide sequence of any one of SEQ ID NOs: 21 to 54.

Embodiment 14d comprises the nucleic acid molecule or combination of embodiment 14b or 14c, in combination with any one of the non-naturally occurring polynucleotide sequences selected from the group consisting of embodiments 14(1), 14(2), 14a1(1), 14a1(2), 14a2(1), and 14a2(2).

Embodiment 14e comprises the nucleic acid molecule or combination of embodiment 14b or 14c, in combination with any one of the non-naturally occurring polynucleotide sequences selected from the group consisting of SEQ ID NOs: 15 to 20.

Embodiment 15 comprises the nucleic acid molecule or combination of embodiment 14, comprising the non-naturally occurring polynucleotide sequence of any one of SEQ ID NOs: 15 to 54.

Embodiment 16 comprises a vector comprising the nucleic acid molecule or combination of any one of embodiments 1-15.

Embodiment 17 comprises the vector of embodiment 16 that is a DNA plasmid.

Embodiment 18 comprises the vector of embodiment 16 that is a DNA viral vector.

Embodiment 18a comprises the vector of embodiment 16 that is an RNA viral vector.

Embodiment 18b comprises the vector of embodiment 18a that is an RNA replicon.

Embodiment 18c comprises the vector of embodiment 16 that is a Modified Vaccinia Ankara (MVA) vector or an adenovirus vector.

Embodiment 18c1 comprises the vector of embodiment 18c that is an MVA-BN vector.

Embodiment 18c2 comprises the vector of embodiment 18c that is an Ad26 or Ad35 vector.

Embodiment 19 comprises an RNA replicon, comprising, ordered from the 5′- to 3′-end:
(1) a 5′ untranslated region (5′-UTR) required for nonstructural protein-mediated amplification of an RNA virus;
(2) a polynucleotide sequence encoding at least one, preferably all, of non-structural proteins of the RNA virus;
(3) a subgenomic promoter of the RNA virus;
(4) the nucleic acid molecule or combination of any one of embodiments 1-15; and
(5) a 3′ untranslated region (3′-UTR) required for nonstructural protein-mediated amplification of the RNA virus.

Embodiment 20 comprises an RNA replicon, comprising, ordered from the 5′- to 3′-end,
(1) an alphavirus 5′ untranslated region (5′-UTR),
(2) a 5′ replication sequence of an alphavirus non-structural gene nsp1, (3) a downstream loop (DLP) motif of a virus species,
(4) a polynucleotide sequence encoding a fourth autoprotease peptide,
(5) a polynucleotide sequence encoding alphavirus non-structural proteins nsp1, nsp2, nsp3 and nsp4,
(6) an alphavirus subgenomic promoter,
(7) the nucleic acid molecule or combination of any one of embodiments 1-15,
(8) an alphavirus 3′ untranslated region (3′ UTR), and
(9) optionally, a poly adenosine sequence.

Embodiment 21 comprises the RNA replicon of embodiment 20, wherein the DLP motif is from a virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MTDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (U AV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus.

Embodiment 22 comprises the RNA replicon of embodiment 21, wherein the fourth autoprotease peptide is selected from the group consisting of porcine tesehovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof.

Embodiment 22a comprises the RNA replicon of embodiment 22, wherein the fourth autoprotease peptide comprises the peptide sequence of P2A.

Embodiment 22b comprises the RNA replicon of embodiment 22 or 22a, wherein the fourth autoprotease peptide comprises the peptide sequence of SEQ ID NO: 11.

Embodiment 23 comprises an RNA replicon, comprising, ordered from the 5′- to 3′-end,
(1) a 5′-UTR having the polynucleotide sequence of SEQ ID NO: 55,
(2) a 5′ replication sequence having the polynucleotide sequence of SEQ ID NO: 56, (3) a DLP motif comprising the polynucleotide sequence of SEQ ID NO: 57,
(4) a polynucleotide sequence encoding a P2A sequence of SEQ ID NO: 11,
(5) polynucleotide sequences encoding alphavirus non-structural proteins nsp1, nsp2, nsp3 and nsp4 encoded by the nucleic acid sequences of SEQ ID NO: 58, SEQ ID NO: 59, SEQ ID NO: 60 and SEQ ID NO: 61, respectively,
(6) a subgenomic promoter having polynucleotide sequence of SEQ ID NO: 62,
(7) the nucleic acid molecule or combination of any one of embodiments 1-15, and (8) a 3′ UTR having the polynucleotide sequence of SEQ ID NO: 63.

Embodiment 24 comprises the RNA replicon of embodiment 23, wherein:
(i) the polynucleotide sequence encoding the P2A sequence comprises SEQ ID NO: 12,
(ii) the polynucleotide sequences encoding the alphavirus non-structural proteins nsp1, nsp2, nsp3 and nsp4 have the nucleic acid sequences of SEQ ID NO: 58, SEQ ID NO: 59, SEQ ID NO: 60 and SEQ ID NO: 61, respectively;
(iii) the nucleic acid molecule or combination comprises the polynucleotide sequence of any one of SEQ ID NOs: 15 to 54, and
(iv) the RNA replicon further comprises a poly adenosine sequence, preferably the poly adenosine sequence has the sequence of SEQ ID NO: 64, at the 3′-end of the replicon.

Embodiment 25 comprises an RNA replicon comprising the polynucleotide sequence of any one of SEQ ID NOs: 65 to 72.

Embodiment 26 comprises a nucleic acid molecule comprising a polynucleotide sequence encoding the RNA replicon of any one of embodiments 19-25, preferably, the nucleic acid further comprises a T7 promoter operably linked to the 5′-end of the DNA sequence, more preferably, the T7 promoter comprises the nucleotide sequence of SEQ ID NO: 73.

Embodiment 27 comprises a pharmaceutical composition comprising the nucleic acid molecule or combination of any one of embodiments 1-15 and 26, the vector of any one of embodiments 16-18c2, or the RNA replicon of any one of embodiments 19-25, and a pharmaceutically acceptable carrier.

Embodiment 28 comprises the pharmaceutical composition of embodiment 27, wherein the pharmaceutically acceptable carrier comprises one or more lipids.

Embodiment 28a comprises the pharmaceutical composition of embodiment 28, wherein the one or more lipids comprise a cationic lipid.

Embodiment 28a1 comprises the pharmaceutical composition of embodiment 28a, wherein the cationic lipid is an ionizable cationic lipid.

Embodiment 28a2 comprises the pharmaceutical composition of embodiment 28a, wherein the cationic lipid is selected from the group consisting of ALC-0315 (((4-hydroxybutyl)azanediyl)bis(hexane-6,1-diyl)bis(2-hexyldecanoate)), DOTMA (N-D-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine), DOPE (1,2-dileoyl-sn-3-phosphoethanolamine), DSDMA, DODMA, DLinDMA, DLenDMA, γ-DLenDMA, DLin-K-DMA, DLin-K-C2-DMA, DLin-K-C3-DMA, DLin-K-C4-DMA, DLen-C2K-DMA, γ-DLen-C2K-DMA, DLin-M-C2-DMA, DLin-M-C3-DMA, DLin-MP-DMA, and DCChol (3 beta-(N—(N′,N′-dimethyl aminomethane)-carbamoyl) cholesterol), and combinations thereof.

Embodiment 28a3 comprises the pharmaceutical composition of any one of embodiments 28a-28a2, wherein the cationic lipid is ALC-0315.

Embodiment 28b comprises the pharmaceutical composition of any one of embodiments 28a-28a3, further comprising one or more of (a) a polyethylene glycol (PEG) lipid or PEG-modified lipid, (b) a helper lipid, and (c) a sterol.

Embodiment 28b1 comprises the pharmaceutical composition of embodiment 28b, wherein the PEG lipid or PEG-modified lipid is selected from the group consisting of 2-[(polyethylene glycol)-2000]-N,N-ditetradecylacetamide (ALC-0159), PEG550-PE, PEG750-PE, PEG2000-DMG, PEG-DSPE, PEG-DAA, PEG-DAG, PEG-PE, monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), and combinations thereof.

Embodiment 28b2 comprises the pharmaceutical composition of embodiment 28b or 28b1, wherein the PEG lipid is ALC-0159.

Embodiment 28c comprises the pharmaceutical composition of any one of embodiments 28b-28b2, wherein the helper lipid is selected from the group consisting of a neutral lipid, neutral helper lipid, non-cationic lipid, non-cationic helper lipid, anionic lipid, anionic helper lipid, or a zwitterionic lipid, or combinations thereof.

Embodiment 28c1 comprises the pharmaceutical composition of embodiment 28c, wherein the helper lipid is selected from the group consisting of distearoylphosphatidylcholine (DSPC), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and combinations thereof.

Embodiment 28c2 comprises the pharmaceutical composition of embodiment 28c or 28c1, wherein the helper lipid is distearoylphosphatidylcholine (DSPC).

Embodiment 28d comprises the pharmaceutical composition of any one of embodiments 28b-28c2, wherein the sterol is selected from the group consisting of cholesterol, 5α-cholestanol, 5α-coprostanol, cholesteryl-(2′-hydroxy)-ethyl ether, cholesteryl-(4′-hydroxy)-butyl ether, 6-ketocholestanol, 5α-cholestane, cholestenone, 5α-cholestanone, 5α-cholestanone, cholesteryl decanoate, and combinations thereof.

Embodiment 28d1 comprises the pharmaceutical composition of embodiment 28d, wherein the sterol is cholesterol.

Embodiment 28e comprises the pharmaceutical composition of any one of embodiments 28-28d1, wherein the one or more lipids comprise ALC-0315, ALC-0159, DSPC, and cholesterol.

Embodiment 28f comprises the pharmaceutical composition of any one of embodiments 28-28e, wherein the pharmaceutically acceptable carrier comprises a lipid nanoparticle.

Embodiment 29 comprises the pharmaceutical composition of any one of embodiments 27-28f, further comprising:
(1) a polynucleotide sequence encoding an HBV core antigen having the amino acid sequence of any one of SEQ ID NOs: 84, 85 or 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen having, preferably consisting of, the amino acid sequence of SEQ ID NO: 9; or
(2) a polynucleotide sequence encoding an HBV polymerase antigen having, preferably consisting of, the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen having the amino acid sequence of any one of SEQ ID NOs: 84, 85 or 86.

Embodiment 30 comprises a method for vaccinating a subject against HBV, the method comprising administering to the subject the pharmaceutical composition according to any one of embodiments 27-28f, preferably the subject has chronic HBV infection.

Embodiment 30a comprises the pharmaceutical composition according to any one of embodiments 27-28f for use in vaccinating a subject against HBV, preferably the subject has chronic HBV infection.

Embodiment 30b comprises the pharmaceutical composition for use according to embodiment 30a, further comprising a second composition comprising a nucleic acid molecule encoding at least one identical HBV epitope, such as at least one identical HLA epitope, preferably at least one identical antigen, for use in a prime-boost regimen.

Embodiment 30c comprises the pharmaceutical composition according to any one of embodiments 27-28f for use in treatment of an HBV infection.

Embodiment 31 comprises the method of embodiment 30, further comprising administering to the subject a second composition comprising a nucleic acid molecule encoding at least one identical HBV epitope, such as at least one identical HLA epitope, preferably at least one identical antigen, in a prime-boost regimen.

Embodiment 32 comprises the method of embodiment 31, wherein the prime-boost regimen comprises a first composition comprising the RNA replicon of any one of embodiments 19-25 and a second composition comprising a vector which is not an RNA replicon, and which encodes at least one identical HBV epitope, such as at least one identical HLA epitope, preferably at least one identical antigen as the priming composition.

Embodiment 32a comprises the method of embodiment 32, wherein the first composition is used for priming immunization and the second composition is used for boosting immunization in the prime-boost regimen.

Embodiment 32b comprises the method of embodiment 32, wherein the second composition is used for priming immunization and the first composition is used for boosting immunization in the prime-boost regimen.

Embodiment 33 comprises the method of any one of embodiments 32-32b, wherein the second composition comprises a Modified Vaccinia Ankara (MVA) vector, an adenovirus vector, or a plasmid.

Embodiment 33a comprises the method of embodiment 33, wherein the second composition comprises an MVA-BN vector.

Embodiment 33b comprises the method of embodiment 33, wherein the second composition comprises an Ad26 or Ad35 vector.

Embodiment 34 comprises a method for reducing infection and/or replication of HBV in a subject, comprising administering to the subject a pharmaceutical composition according to any one of embodiments 27-28f, or vaccinating the subject according to any one of embodiments 30 or 31-33b.

Embodiment 34a comprises the method according to any one of embodiments 30 or 31-34, wherein the subject is selected from the group consisting of HLA-A*11:01 subjects, HLA-A*24:02 subjects, HLA-A*02:01 subjects, HLA-A*A2402 subjects, HLA-A*A0101 subjects and HLA-B*40:01 subjects.

Embodiment 34a1 comprises the method according to embodiment 34a, wherein the subject is selected from the group consisting of HLA-A*11:01 subjects, HLA-A*24:02 subjects, HLA-A*02:01 subjects and HLA-A*A2402 subjects.

Embodiment 34a2 comprises the method according to embodiment 34a or 34a1, wherein the subject is selected from the group consisting of HLA-A*11:01 subjects, HLA-A*24:02 subjects and HLA-A*02:01 subjects.

Embodiment 34a3 comprises the method according to any one of embodiments 34a-34a2, wherein the subject is an HLA-A*11:01 subject.

Embodiment 34a4 comprises the method according to any one of embodiments 34a-34a3, wherein the subject is a human patient.

Embodiment 34b comprises the method according to any one of embodiments 30 or 31-34, wherein the subject comprises at least one HBV antigen containing one or more epitopes selected from the group consisting of HLA-A*11:01 epitopes, HLA-A*24:02 epitopes, HLA-A*02:01 epitopes, HLA-A*A2402 epitopes, HLA-A*A0101 epitopes and HLA-B*40:01 epitopes.

Embodiment 34b1 comprises the method according to embodiment 34b, wherein the subject comprises at least one HBV antigen containing one or more epitopes selected from the group consisting of HLA-A*11:01 epitopes, HLA-A*24:02 epitopes, HLA-A*02:01 epitopes and HLA-A*A2402 epitopes.

Embodiment 34b2 comprises the method according to embodiment 34b or 34b1, wherein the subject comprises at least one HBV antigen containing one or more epitopes selected from the group consisting of HLA-A*11:01 epitopes, HLA-A*24:02 epitopes and HLA-A*02:01 epitopes.

Embodiment 34b3 comprises the method according to any one of embodiments 34b-34b2, wherein the subject comprises at least one HBV antigen containing one or more HLA-A*11:01 epitopes.

Embodiment 34b4 comprises the method according to any one of embodiments 34b-34b3, wherein the subject is a human patient.

Embodiment 35 comprises an isolated host cell comprising the nucleic acid molecule or combination of any one of embodiments 1-15 and 26, the vector of any one of embodiments 16-18c2, or the RNA replicon of any one of embodiments 19-25.

Embodiment 36 comprises a method of producing an RNA replicon, comprising transcribing the nucleic acid according to embodiment 26 in vivo or in vitro.

Embodiment 36a comprises the method of embodiment 36, wherein the nucleic acid is transcribed in vivo in a non-human animal.

Embodiment 36b comprises the method of embodiment 36, wherein the nucleic acid is transcribed in vivo in a human.

Embodiment 37 comprises the pharmaceutical composition of any one of embodiments 27-28f for use in inducing an immune response against a hepatitis B virus (HBV) in a subject in need thereof, preferably the subject has chronic HBV infection, optionally in combination with another immunogenic agent or other anti-HBV agent.

Embodiment 37a comprises the pharmaceutical composition for use according to embodiment 37, wherein the other anti-HBV agent is a small molecule, an antibody or antigen binding fragment thereof, a polypeptide, protein, or nucleic acid.

Embodiment 37b comprises a method of inducing an immune response against a hepatitis B virus (HBV) in a subject in need thereof, comprising administering to the subject the pharmaceutical composition of any one of embodiments 27-28f, optionally the method further comprising administering to the subject another immunogenic agent and/or another anti-HBV agent, preferably the subject has chronic HBV infection.

Embodiment 38 comprises the pharmaceutical composition of any one of embodiments 27-28f for use in treating a hepatitis B virus (HBV)-induced disease in a subject in need thereof, preferably the subject has chronic HBV infection, and the HBV-induced disease is selected from the group consisting of advanced fibrosis, cirrhosis and hepatocellular carcinoma (HCC), optionally in combination with another therapeutic agent, preferably another anti-HBV agent.

Embodiment 38a comprises a method of treating a hepatitis B virus (HBV)-induced disease in a subject in need thereof, comprising administering to the subject the pharmaceutical composition of any one of embodiments 27-28f, optionally the method further comprising administering to the subject another therapeutic agent, preferably another anti-HBV agent, preferably the subject has chronic HBV infection and the HBV-induced disease is selected from the group consisting of advanced fibrosis, cirrhosis and hepatocellular carcinoma (HCC).

Embodiment 39 comprises an isolated HBV antigen comprising the amino acid sequence of any one of SEQ ID NOs: 1, 3, 5, 9, 84, 85, or 86.

Embodiment 39a comprises the isolated HBV antigen of embodiment 39, wherein the HBV antigen comprises a consensus sequence for HBV genotypes A, B, C and D.

Embodiment 39b comprises the isolated HBV antigen of embodiment 39 or 39a, wherein the HBV antigen comprises at least two, three, four, five or all of the epitopes for HLA-A*11:01, HLA-A*24:02, HLA-A*02:01, HLA-A*A2402, HLA-A*A0101 and HLA-B*40:01.

Embodiment 39c comprises the isolated HBV antigen of any one of embodiments 39-39b, consisting of the amino acid sequence of any one of SEQ ID NOs: 1, 3, 5, 9, 84, 85, or 86.

Embodiment 40 comprises an isolated polynucleotide sequence encoding the HBV antigen of any one of embodiments 39-39c.

Embodiment 40a comprises the isolated polynucleotide sequence of embodiment 40 comprising the nucleotide sequence of any one of SEQ ID NOs: 2, 4, 6, 10, 87, 88, or 89.

Embodiment 40b comprises the isolated polynucleotide sequence of embodiment 40a consisting of the nucleotide sequence of any one of SEQ ID NOs: 2, 4, 6, 10, 87, 88, or 89.

Embodiment 41 is a vector comprising the polynucleotide sequence of any one of embodiments 40-40b.

Embodiment 41a is the vector of embodiment 41, which is a plasmid.

Embodiment 41b is the vector of embodiment 41, which is a viral vector.

Embodiment 41b1 is the vector of embodiment 41b, which is an MVA vector, preferably MVA-BN.

Embodiment 41b2 is the vector of embodiment 41b, which is an adenoviral vector, preferably Ad26 or Ad35.

Embodiment 41c is the vector of embodiment 41, which is an RNA replicon.

Embodiment 42 is a pharmaceutical composition comprising the isolated HBV antigen of any one of embodiments 39-39c, the isolated polynucleotide sequence of any one of embodiments 40-40b, or the vector of any one of embodiments 41-41c.

Embodiment 43 is a method of inducing an immune response in a subject in need thereof, comprising administering to the subject the pharmaceutical composition of embodiment 42.

Embodiment 43a comprises the pharmaceutical composition of embodiment 42 for use in inducing an immune response against a hepatitis B virus (HBV) in a subject in need thereof, preferably the subject has chronic HBV infection, optionally in combination with another immunogenic agent, preferably another anti-HBV agent.

Embodiment 44 comprises an isolated host cell comprising the nucleic acid molecule of any one of embodiments 40-40b, the vector of any one of embodiments 41-41c.

### EXAMPLES

### Example 1: Antigen Selection, Design and In Vitro Evaluation of Replicon Vaccine Candidates

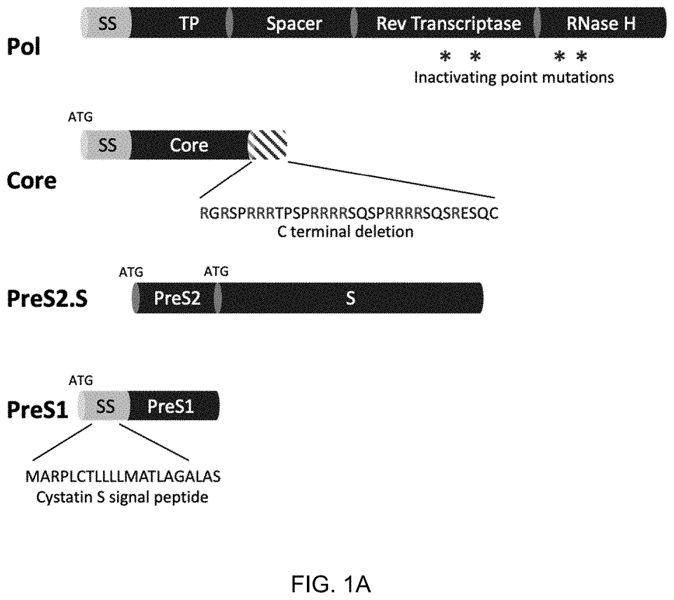

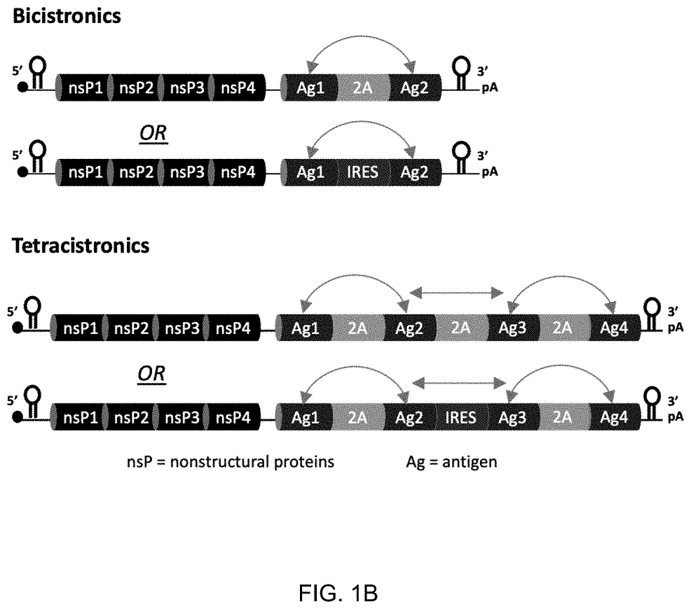

The highly immunogenic HBV proteins Core, Pol, PreS2.S and PreS1 domain from L surface antigen were each selected for inclusion in a replicon HBV therapeutic vaccine. To centralize immunogenicity, a consensus sequence was generated based on the alignments of unique sequences for each antigen from genotypes A, B, C and D. By including these four genotypes, which make up >78% of the world's chronic hepatitis B (CHB) infections, the size of the treatable target population is maximized. Known human T cell epitopes for the top 3 most common MHC class I HLA alleles in China, the United States and Europe (including HLA-A*11:01, HLA-A*24:02, HLA-A*02:01, HLA-A*A0201, HLA-A*A2402, HLA-A* A0101, and HLA-B*40:01) were mapped to each consensus sequence. If a known epitope was found to be altered, the consensus sequence was adjusted to restore the epitope. For example, Arg149, 150 and 151 of the C terminus was included in HBV Core encoded in the HBV therapeutic vaccine. Pol was further optimized to inactivate its reverse transcriptase and RNase H activity. The Cystatin S precursor signal peptide was added to Core, Pol and the PreS1 domain to enhance secretion, while the internal signal peptide of PreS2.S was left intact to facilitate secretion PreS2.S protein products M and S. Finally, the amino acid sequences for each antigen were reverse translated and codon optimized to maximize expression in humans (FIG. 1A).

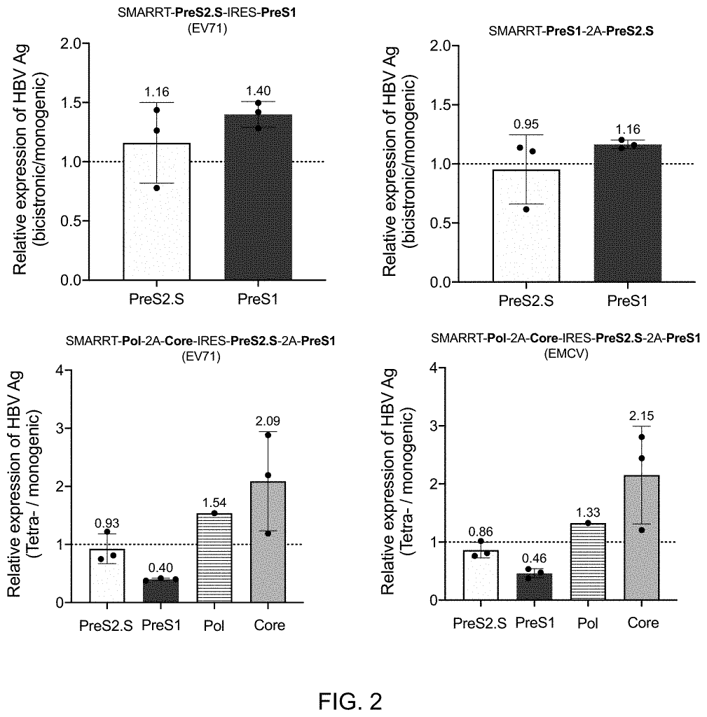

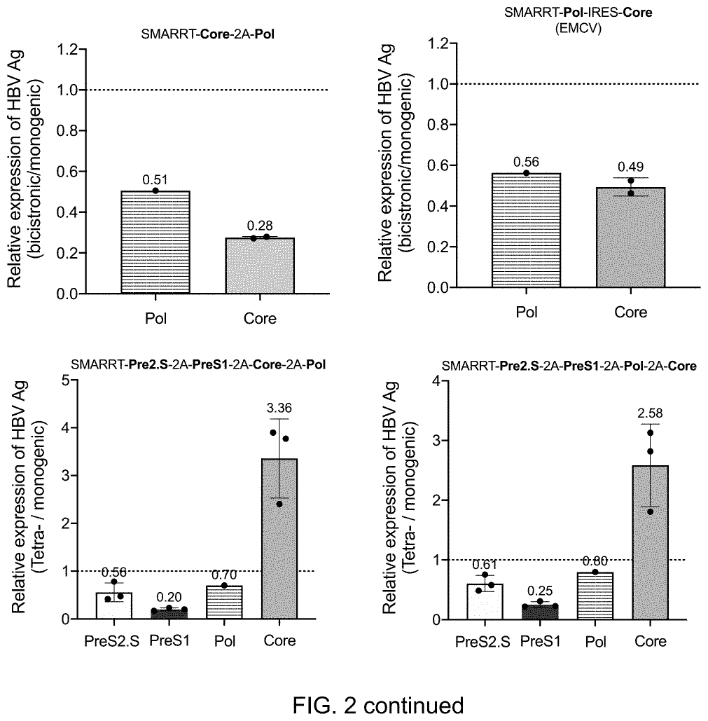

These antigens were encoded either in the replicon alone or in multicistronic configurations, with different positioning of each antigen within the replicon, each linked by a P2A ribosomal skipping element or an Internal Ribosomal Entry Site (IRES) from EMCV or EV71 (FIG. 1B). Using a plasmid template, RNA for each replicon design was produced in an in vitro transcription reaction and electroporated into Vero cells. 24-48 hours post electroporation, expression and secretion of each antigen was measured by flow cytometry, Western blot analysis, and ELISA. The frequency of double stranded RNA (dsRNA) and antigen-positive cells were also assayed as an indicator of replicon amplification efficiency. For each construct, every antigen was given a score based on the relative level of 1) expression, 2) secretion and 3) frequency of antigen/dsRNA positive cells relative to a replicon expressing the single antigen. The antigen scores were weighted and combined to give a total replicon construct score (Table 1, below). Each construct was ranked based on these scores and the top 4 bicistronic and top 4 tetracistronic constructs were selected to advance to in vivo evaluation (FIG. 2). In case of tie construct scores, gene scores were prioritized in the following order used to rank these constructs: Core>Pol>PreS2.S>PreS1. In addition, a construct with more similar relative expression levels of each antigen was prioritized over a construct that had high expression of a single antigen and low expression of the other antigens.

TABLE 1

| | | | | | Total | |
|---|---|---|---|---|---|---|
| | Core | Pol | PreS2.S | PreS1 | Construct | |
| | Score | Score | Score | Score | Score | Max |
| Projected weighted average | 0.40 | 0.25 | 0.20 | 0.15 | 1 | score |
| Core | (1 × 0.4) = 0.4 | 0 | 0 | 0 | 0.4 | 0.4 |
| Pol | 0 | 0.25 | 0 | 0 | 0.25 | 0.25 |
| preS2.S | 0 | 0 | 0.20 | 0 | 0.20 | 0.20 |
| preS1 | 0 | 0 | 0 | 0.1 | 0.1 | 0.1 |
| Core-2A-Pol | 0.22 | 0.16 | — | — | 0.39* | 0.65 |
| Pol-2A-Core | 0.17 | 0.24 | — | — | 0.41 | 0.65 |
| preS2.S-2A-preS1 | — | — | 0.145 | 0.150 | 0.295 | 0.35 |
| preS1-2A-preS2-S | — | — | 0.188 | 0.150 | 0.338 | 0.35 |
| Core-IRES-Pol (EMCV IRES) | 0.18 | 0.20 | — | — | 0.39 | 0.65 |
| Pol-IRES-Core (EMCV IRES) | 0.32 | 0.17 | — | — | 0.50 | 0.65 |
| Core-IRES-Pol (EV71 IRES) | 0.19 | 0.20 | — | — | 0.38 | 0.65 |
| Pol-IRES-Core (EV71 IRES) | 0.28 | 0.18 | — | — | 0.46 | 0.65 |
| preS2.S-IRES-preS1 (EMCV IRES) | — | — | 0.189 | 0.150 | 0.339 | 0.35 |
| preS1-IRES-preS2.S (EMCV IRES) | — | — | 0.119 | 0.150 | 0.269 | 0.35 |
| preS2.S-IRES-preS1 (EV71 IRES) | — | — | 0.191 | 0.150 | 0.341 | 0.35 |
| preS1-IRES-preS2.S (EV71 IRES) | — | — | 0.158 | 0.150 | 0.308 | 0.35 |
| Core-2A-Pol-2A-preS2.S-2A-preS1 | 0.305 | 0.137 | 0.057 | 0.013 | 0.51 | 1 |
| Pol-2A-Core-2A-preS2.S-2A-preS1 | 0.281 | 0.232 | 0.062 | 0.016 | 0.59 | 1 |
| preS2.S-2A-preS1-2A-Core-2A-Pol | 0.282 | 0.171 | 0.093 | 0.042 | 0.59 | 1 |
| preS2.S-2A-preS1-2A-Pol-2A-Core | 0.287 | 0.196 | 0.099 | 0.052 | 0.63 | 1 |
| Core-2A-Pol-IRES-preS2.S-2A-preS1 (EMCV IRES) | 0.290 | 0.170 | 0.126 | 0.096 | 0.68 | 1 |
| Pol-2A-Core-IRES-preS2.S-2A-preS1 (EMCV IRES) | 0.276 | 0.242 | 0.121 | 0.079 | 0.72 | 1 |
| preS2.S-2A-preS1-IRES-Core-2A-Pol (EMCV IRES) | 0.272 | 0.218 | 0.104 | 0.029 | 0.62 | 1 |
| preS2.S-2A-preS1-IRES-Pol-2A-Core (EMCV IRES) | 0.287 | 0.250 | 0.112 | 0.037 | 0.69 | 1 |
| Core-2A-Pol-IRES-preS2.S-2A-preS1 (EV71 IRES) | 0.259 | 0.128 | 0.118 | 0.069 | 0.57 | 1 |
| Pol-2A-Core-IRES-preS2.S-2A-preS1 (EV71 IRES) | 0.258 | 0.233 | 0.121 | 0.069 | 0.68* | 1 |
| preS2.S-2A-preS1-IRES-Core-2A-Pol (EV71 IRES) | 0.286 | 0.243 | 0.103 | 0.029 | 0.66 | 1 |
| preS2.S-2A-preS1-IRES-Pol-2A-Core (EV71 IRES) | 0.265 | 0.249 | 0.101 | 0.030 | 0.65 | 1 |

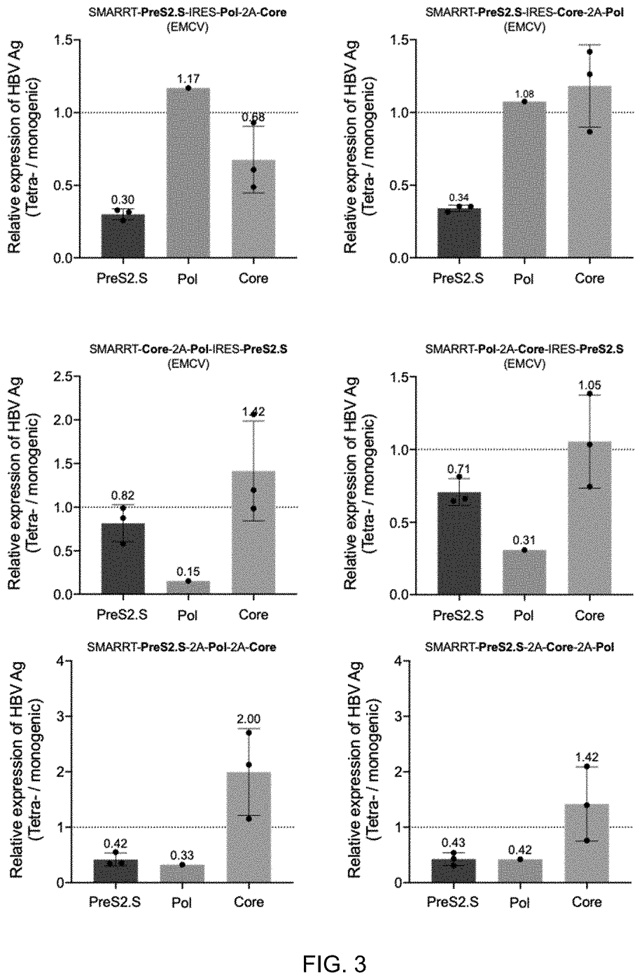

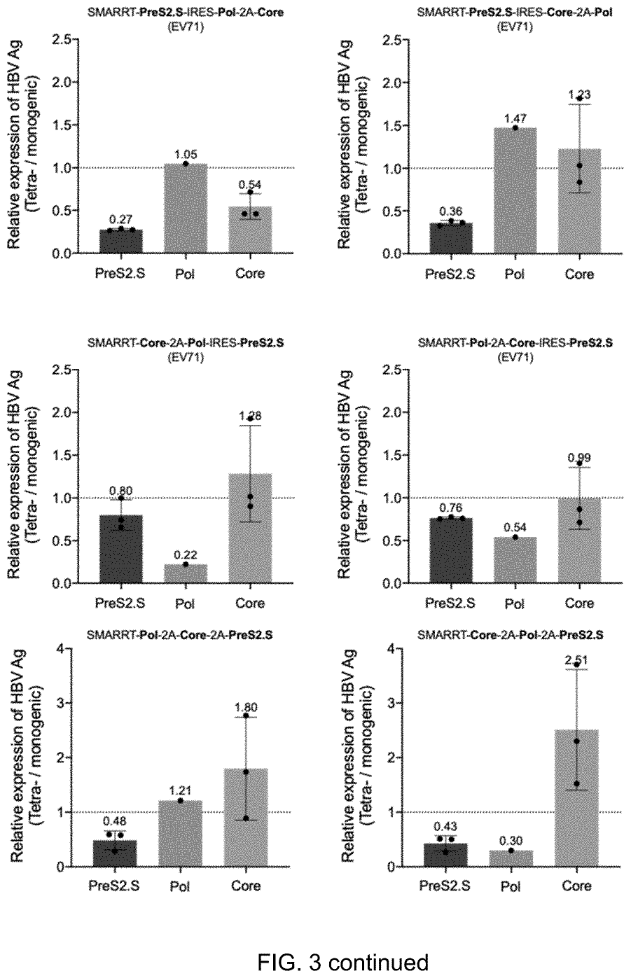

FIG. 2 shows the expression of each antigen from the top 8 replicons relative to the monogenic controls. All constructs were able to maintain relatively high levels of expression of each antigen. However, when core and pol were expressed from the same bicistronic replicon RNA, a decrease in Core expression was observed. In contrast, tetracistronic replicons expressing Core, Pol, PreS2.S and PreS1 from the same replicon consistently demonstrated a 2-3 fold increase in Core expression relative to a monogenic control. Expression of Core, Pol and PreS2.S from a tricistronic replicon RNA also resulted in an increase in Core expression levels (FIG. 3), although the increase was not as dramatic as when PreS1 was expressed from the same replicon RNA.

### Example 2: SMARRT Replicon RNA Platform Induces Cellular Responses in Mice to HBV Targets

The purpose of these studies was to determine if Synthetic Modified Alpha RNA replicon technology (SMARRT) encoding HBV antigens could prime immune responses in C57BL/6 mice. SMARRT monogenic HBV constructs were dosed at 15 μg and the admixed group received 4 SMARRT monogenic replicons at 15 μg of each replicon (total of 60 μg RNA per mouse). At Week 0, mice were immunized by IM injection with the indicated SMARRT construct(s), and a control group was injected with saline. At Week 2, all animals were sacrificed and splenocytes were stimulated with 15-mer overlapping peptide pools covering the antigen sequence in the insert (for SMARRT.Pol the overlapping library was split into 2 pools). The induction of IFN-γ-producing cells was measured by IFN-γ ELISpot. CD8 and CD4 polyfunctional T cell responses were determined by measuring the production of IFN-γ, TNFα and IL-2 by flow cytometry. Table 2 below shows the various experimental groups.

TABLE 2

| Group | Animal # | Description of groups |
|---|---|---|
| 1 | 5 | Saline |
| 2 | 5 | SMARRT.Core |
| 3 | 5 | SMARRT.Pol |
| 4 | 5 | SMARRT.PreS2.S |
| 5 | 5 | SMARRT.PreS1 |
| 6 | 5 | SMARRT.Admixed monotopes |

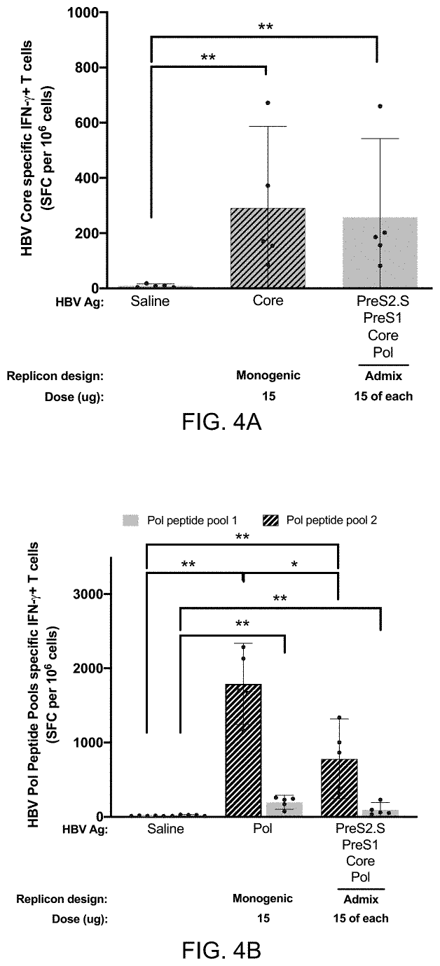

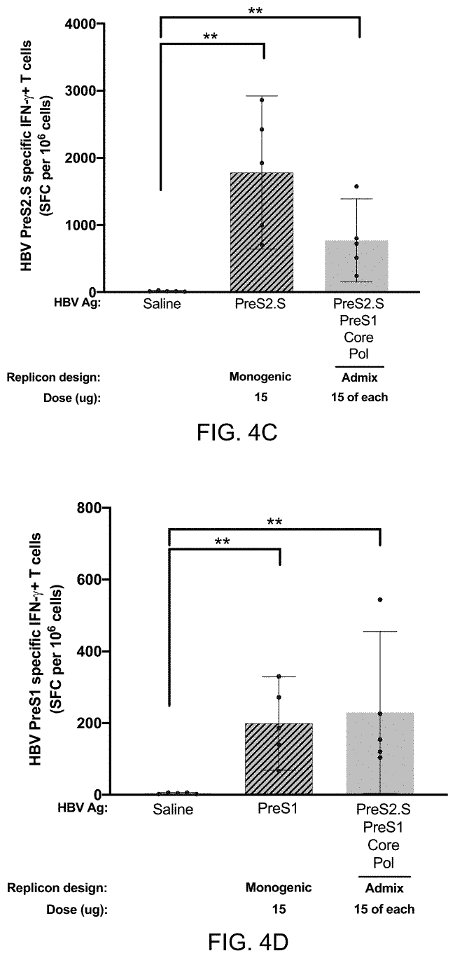

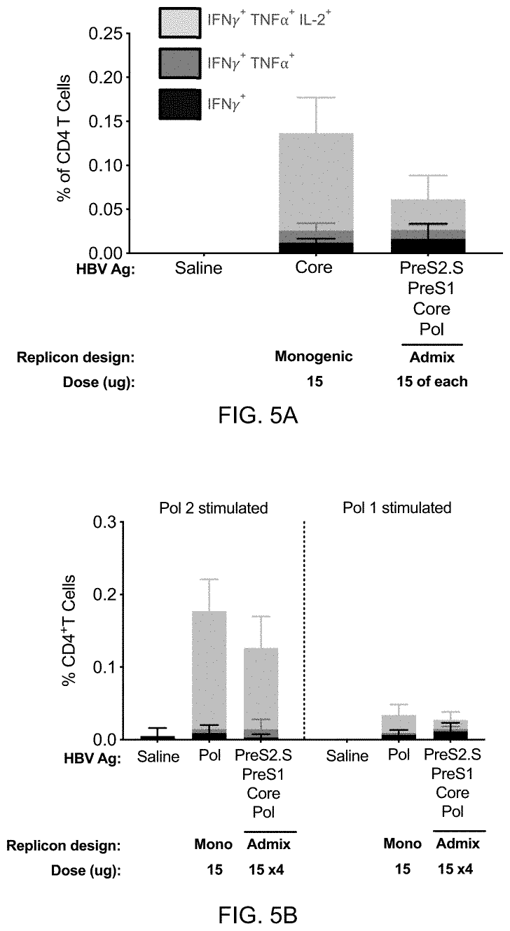

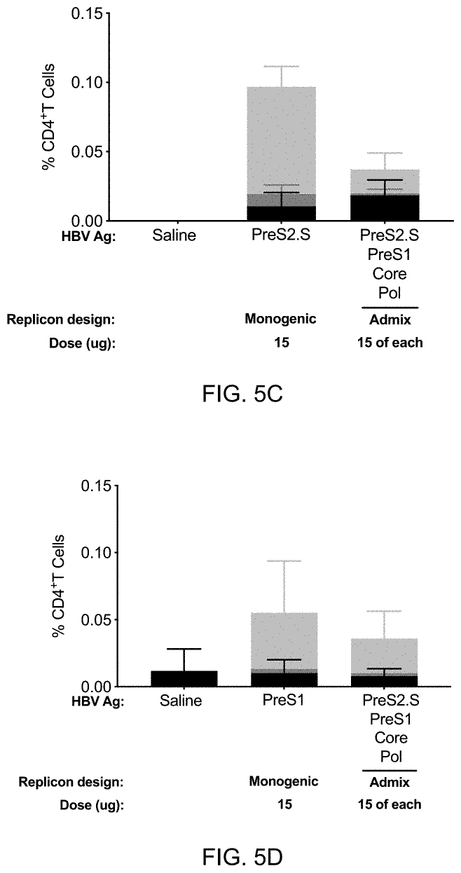

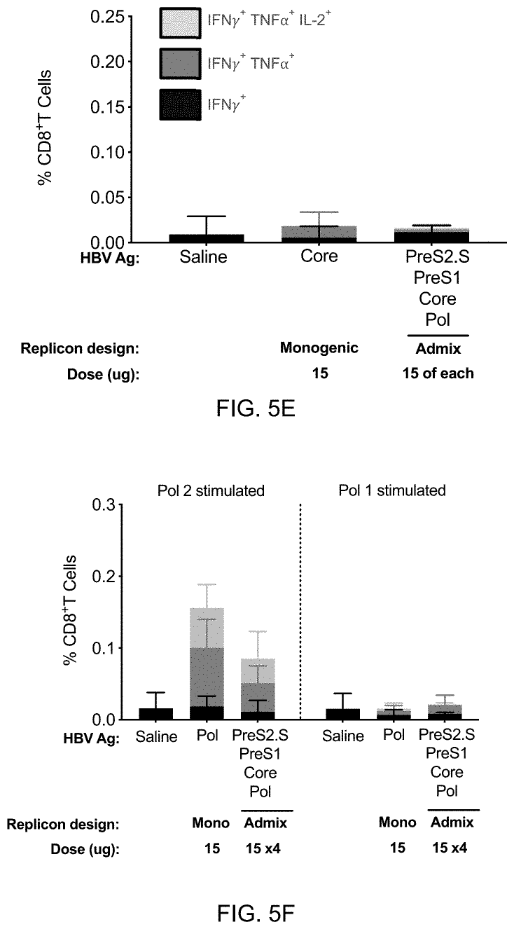

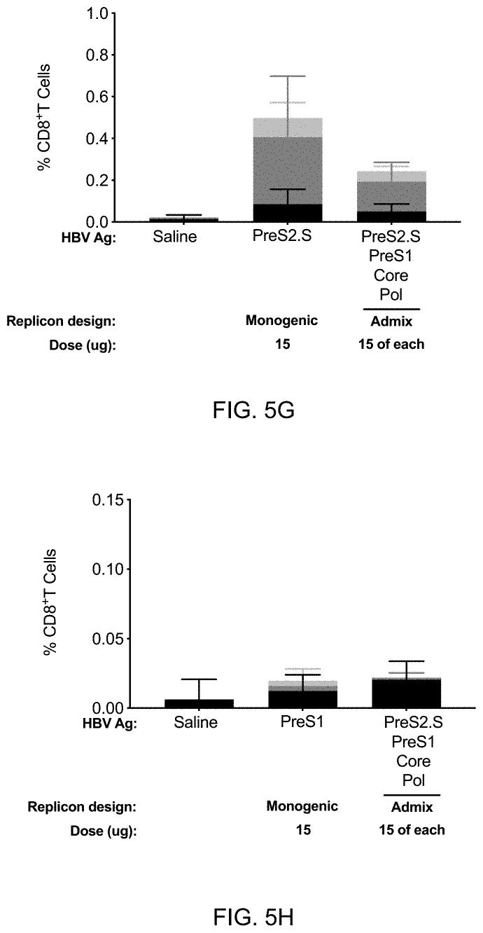

All animals immunized with SMARRT replicon-encoding HBV antigens developed IFN-γ-producing cells upon stimulation with peptides covering the appropriate antigen sequence (FIG. 4). In addition, admixing the 4 SMARRT replicons induced IFNγ producing cells to all 4 antigens. In an identical, but separate experiment, polyfunctional T cell cytokine production was measured by intracellular flow cytometry (FIG. 5). This experiment showed that immunization of mice with SMARRT.Core or SMARRT.PreS1 induced polyfunctional CD4 T cells in C57BL/6 mice. SMARRT.Pol and SMARRT.PreS2.S immunization resulted in both polyfunctional CD4 and CD8 T cell responses in mice.

### Example 3: Down Selection of SMARRT HBV Therapeutic Vaccine Candidates in Mice

Following in vitro screening, 8 constructs were selected for in vivo immunogenicity analysis in mice, this included 4 tetracistronic constructs and 4 bigenic constructs which were admixed in 4 combinations to deliver all 4 HBV antigens. C57BL/6 mice were immunized on Week 0, then spleens harvested on Week 2 for immunogenicity analysis according to the experimental outline in Table 3 below. Ex vivo studies included IFNγ ELISpot and intracellular cytokine staining

TABLE 3

| Group | Animal # | Description of groups (2-15 all SMARRT) |
|---|---|---|
| 1 | 5 | Saline |
| 2 | 5 | SMARRT.Core |
| 3 | 5 | SMARRT.Pol |
| 4 | 5 | SMARRT.PreS2.S |
| 5 | 5 | SMARRT.PreS1 |
| 6 | 5 | SMARRT.Admixed monogenics |
| 7 | 5 | PreS2.S-IRES-PreS1 (EV71 IRES) + Pol-IRES-Core (EMCV IRES) Admixed |
| 8 | 5 | PreS2.S-IRES-PreS1 (EV71 IRES) + Core-2A-Pol Admixed |
| 9 | 5 | PreS1-2A-PreS.S + Pol-IRES-Core (EMCV IRES) Admixed |
| 10 | 5 | PreS1-2A-PreS.S + Core-2A-Pol Admixed |
| 11 | 5 | PreS1-2A-PreS.S-2A-Core-Pol |
| 12 | 5 | PreS1-2A-PreS.S-2A-Pol-2A-Core |
| 13 | 5 | Pol-2A-Core-IRES-PreS2.S-2A-PreS1 (EMCV IRES) |
| 14 | 5 | Pol2A-Core-IRES-PreS2.S-2A-PreS1 (EV71 IRES) |
| 15 | 5 | SMARRT.Core + Pol admixed |

All bigenic and tetracistronic constructs induced a strong T cell response against HBV Core (FIG. 6A), Pol (FIG. 6B), and PreS2.S (FIG. 6C), as measured by IFNγ ELISpot. All bigenic and tetracistronic constructs induced a T cell response against PreS1 (FIG. 6D), with some variability depending on the construct. The strongest PreS1 responses were induced by the admixtures of bigenic 2 and 3 (Group 9), bigenic 2 and 4 (Group 10), and the tetracistronic constructs (Groups 11-14).

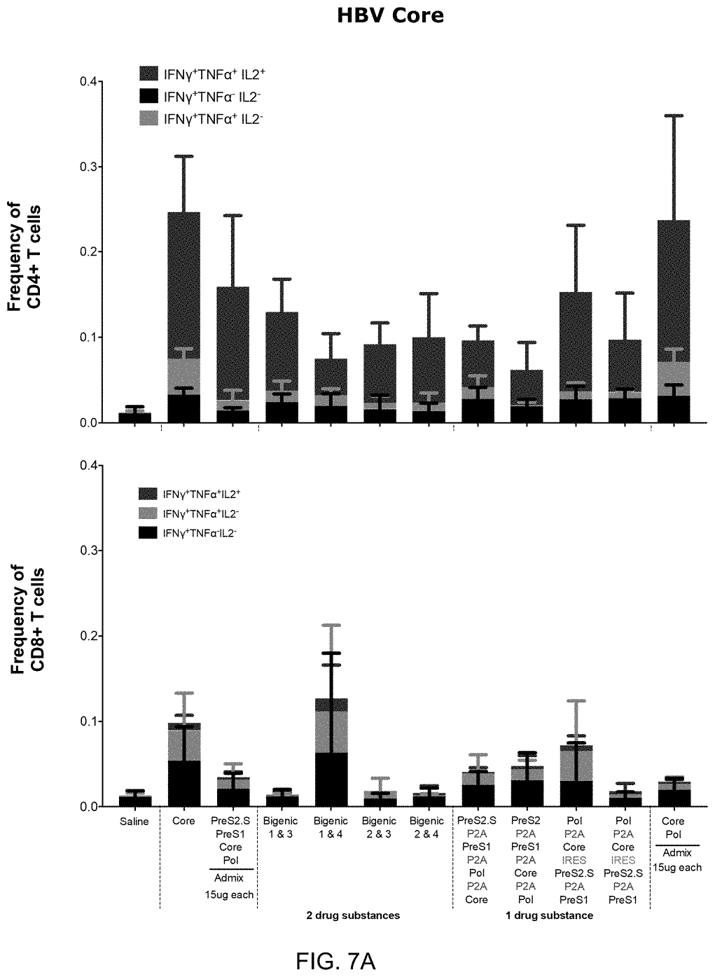

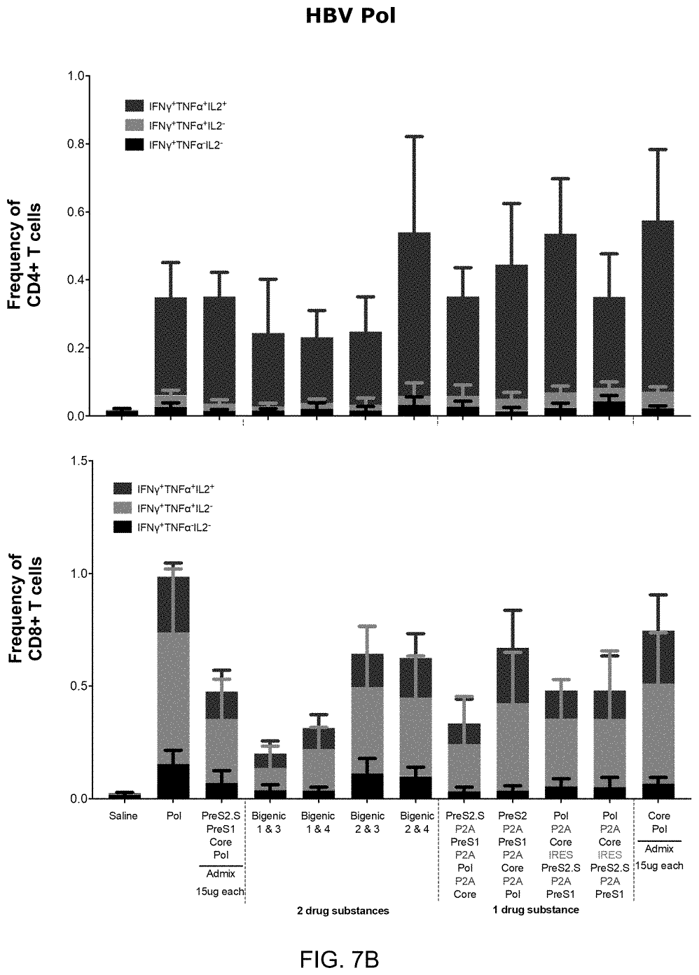

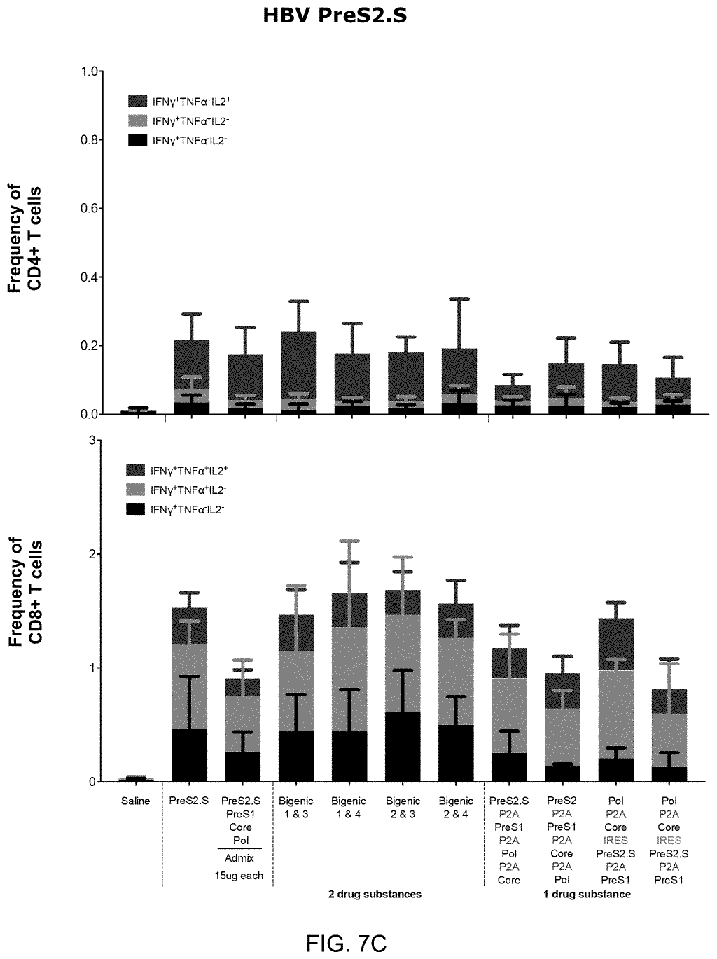

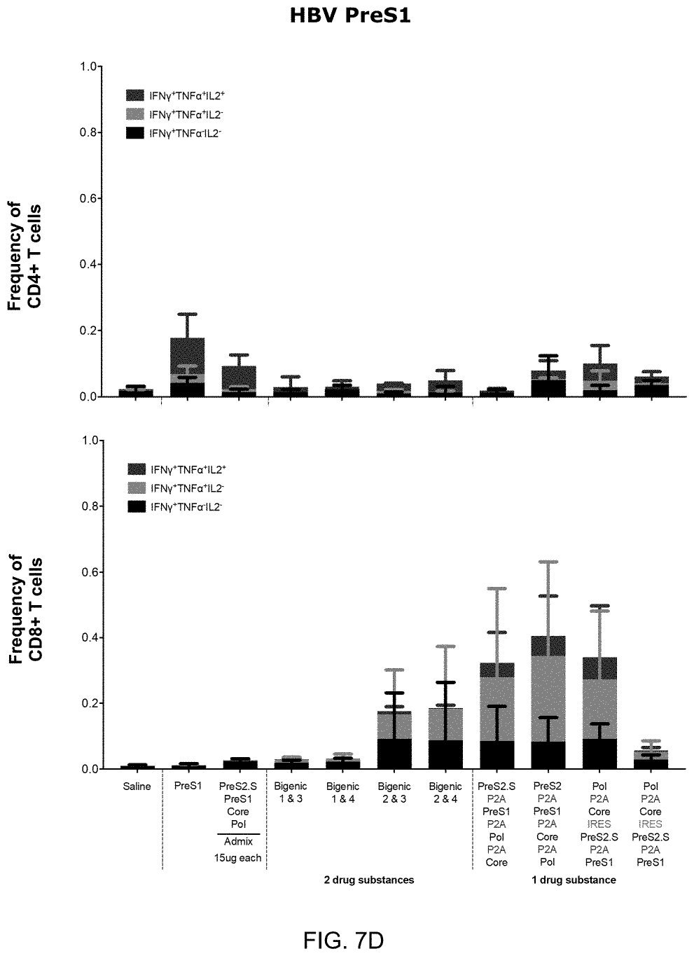

All bigenic and tetracistronic constructs induced polyfunctional CD4+ and CD8+ T cell responses against Core (FIG. 7A), Pol (FIG. 7B), PreS2.S (FIG. 7C), and PreS1 (FIG. 7D), as measured by the ability to produce multiple cytokines. The responses against PreS1 were more variable, depending on the construct, with the strongest responses induced by the admixtures of bigenic 2 and 3 (Group 9), bigenic 2 and 4 (Group 10), and the tetracistronic constructs (Groups 11-14).

### Example 4: Down Selection of SMARRT HBV Therapeutic Vaccine Candidates in Non-Human Primates

To confirm the selected constructs chosen in mice are immunogenic in large animals, an immunogenicity study will be performed in cynomolgous macaques (M. fascicularis). NHPs will be immunized with 2 different SMARRT vaccine candidates at Week 0 then boosted every 4 weeks an additional 3 times. 3 different dose levels of SMARRT will be assessed as shown in Table 4, below. Ex vivo assays will include IFNγ ELISpot and intracellular cytokines staining using PBMC to determine functional T cell responses at 10 days post-injection. Serum will be taken on the injection days to measure anti-HBsAg antibody levels by ELISA. Serum will also be collected on the day of injection, as well as 6 hours post-injection and 24 hours post-injection to measure cytokines and C-reactive protein levels.

TABLE 4

| Group | Animal # | Description of groups |
|---|---|---|
| 1 | 5 (3m, 2f) | Saline |
| 2 | 5 (3m, 2f) | SMARRT candidate 1 100 μg |
| 3 | 5 (3m, 2f) | SMARRT candidate 1 30 μg |
| 4 | 5 (3m, 2f) | SMARRT candidate 1 10 μg |
| 5 | 5 (3m, 2f) | SMARRT candidate 2 100 μg |
| 6 | 5 (3m, 2f) | SMARRT candidate 2 30 μg |
| 7 | 5 (3m, 2f) | SMARRT candidate 2 10 μg |
| 8 | 5 (3m, 2f) | SMARRT.Admixed monotopes |

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Other embodiments are within the following claims.

1. A nucleic acid combination comprising a first non-naturally occurring polynucleotide sequence comprising, ordered from the 5′- to 3′-end:
(1) a polynucleotide sequence encoding a first hepatitis B virus (HBV) antigen,
(2) a first internal ribosome entry sequence (IRES) element or a polynucleotide sequence encoding a first autoprotease peptide, and
(3) a polynucleotide sequence encoding a second HBV antigen, and
a second non-naturally occurring polynucleotide sequence comprising, ordered from the 5′- to 3′-end:
(1) a polynucleotide sequence encoding a third hepatitis B virus (HBV) antigen,
(2) a second internal ribosome entry sequence (IRES) element or a polynucleotide sequence encoding a second autoprotease peptide, and
(3) a polynucleotide sequence encoding a fourth HBV antigen,
wherein the first and second non-naturally occurring polynucleotide sequence are linked by a third internal ribosome entry sequence (IRES) element or a polynucleotide sequence encoding a third autoprotease peptide, or are present in separate nucleic acid molecules, and
wherein the first, second, third and fourth HBV antigens are each independently selected from the group consisting of an HBV core antigen, an HBV polymerase (pol) antigen, and an HBV surface antigen, and at least one of the first, second, third and fourth HBV antigens is an HBV surface antigen selected from an HBV Pre-S1 antigen having an amino acid sequence at least 98% identical to the amino acid sequence of SEQ ID NO: 1 or SEQ ID NO: 3 and an HBV PreS2.S antigen having an amino acid sequence at least 98% identical to the amino acid sequence of SEQ ID NO: 5.

2. The nucleic acid combination of claim 1, wherein one of the first, second, third or fourth HBV antigens is an HBV core antigen, and one is an HBV pol antigen.

3. The nucleic acid combination of claim 1, wherein each of the first, second, third and fourth HBV antigens is different from each other.

4. The nucleic acid combination of claim 1, wherein each of the first, second, third and fourth HBV antigens is independently selected from the group consisting of:
(i) a first HBV Pre-S1 antigen comprising an amino acid sequence that is at least 98% identical to the amino acid sequence of SEQ ID NO: 1, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 1;
(ii) a second HBV Pre-S1 antigen comprising amino acid sequence that is at least 98% identical to the amino acid sequence of SEQ ID NO: 3, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 3;
(iii) an HBV PreS2.S antigen comprising an amino acid sequence that is at least 98% identical to the amino acid sequence of SEQ ID NO: 5, such as at least 98%, 98.5%, 99%, 99.1%, 99.2%, 99.3%, 99.4%, 99.5%, 99.6%, 99.7%, 99.8%, 99.9% or 100% identical to the amino acid sequence of SEQ ID NO: 5;
(iv) an HBV core antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7; and
(v) an HBV pol antigen comprising an amino acid sequence that is at least 90% identical to SEQ ID NO: 9, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 9,
where each of the first and second HBV Pre-S1 antigens, the HBV core antigen and the HBV pol antigen is independently operably linked to a signal peptide, and the HBV PreS2.S antigen comprises an internal signal peptide.

5. The nucleic acid combination of claim 1, wherein the HBV core antigen comprises an amino acid sequence that is at least 98% identical to at least one of SEQ ID NOs: 84, 85, or 86, such as at least 98%, at least 99%, or 100% identical to SEQ ID NOs: 84, 85, or 86.

6. The nucleic acid combination of claim 1, wherein the last five C-terminal amino acids of the HBV core antigen comprise a VVR amino acid sequence, more particularly a VVRR (SEQ ID NO: 91) amino acid sequence, more particularly a VVRRR (SEQ ID NO: 92) amino acid sequence.

7. The nucleic acid combination of claim 1, wherein each of the HBV surface antigen, the HBV core antigen and the HBV pol antigen comprises:
(i) a consensus sequence for HBV genotypes A, B, C and D; and/or
(ii) one or more epitopes for HLA-A*11:01, HLA-A*24:02, HLA-A*02:01, HLA-A*A2402, HLA-A*A0101, or HLA-B*40:01.

8. The nucleic acid combination of claim 7, wherein each of the HBV surface antigens, the HBV core antigen and the HBV pol antigen comprises one or more epitopes for HLA-A*11:01.

9. The nucleic acid combination of claim 4, wherein each of the first, second, third and fourth HBV antigens is independently selected from the group consisting of:
(i) the first HBV Pre-S1 antigen consisting of the amino acid sequence of SEQ ID NO: 1;
(ii) the second HBV Pre-S1 antigen consisting of the amino acid sequence of SEQ ID NO: 3;
(iii) the HBV PreS2.S antigen consisting of the amino acid sequence of SEQ ID NO: 5;
(iv) the HBV core antigen consisting of the amino acid sequence of SEQ ID NO: 84, SEQ ID NO: 85, or SEQ ID NO: 86; and
(v) the HBV pol antigen consisting of the amino acid sequence of SEQ ID NO: 9;
where each of the first and second HBV Pre-S1 antigens, the HBV core antigen and the HBV pol antigen is independently operably linked to a signal peptide, such as the signal peptide comprising the amino acid sequence of SEQ ID NO: 77.

10. The nucleic acid combination of claim 1, wherein each of the polynucleotide sequences encoding the first, second, third and fourth HBV antigens is independently selected from the group consisting of:
(i) a polynucleotide sequence encoding the first HBV Pre-S1 antigen having a sequence that is at least 90% identical to SEQ ID NO: 2, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2;
(ii) a polynucleotide sequence encoding the second HBV Pre-S1 antigen having a sequence that is at least 90% identical to SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4;
(iii) a polynucleotide sequence encoding the HBV PreS2. S antigen having a sequence that is at least 90% identical to SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 6;
(iv) a polynucleotide sequence encoding the HBV core antigen having a sequence that is at least 90% identical to SEQ ID NO: 8, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 8; and
(v) the polynucleotide sequence encoding the HBV pol antigen having a sequence that is at least 90% identical to SEQ ID NO: 10, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 10;
where the polynucleotide sequence encoding each of the first and second HBV Pre-S1 antigens, the HBV core antigen and the HBV pol antigen is independently operably linked to a polynucleotide sequence encoding a signal peptide, and the HBV PreS2.S antigen comprises an internal signal peptide.

11. The nucleic acid combination of claim 10, wherein each of the polynucleotide sequences encoding the first, second, third and fourth HBV antigens is independently selected from the group consisting of:
(i) a polynucleotide sequence encoding the first HBV Pre-S1 antigen consisting of the sequence of SEQ ID NO: 2;
(ii) a polynucleotide sequence encoding the second HBV Pre-S1 antigen consisting of the sequence of SEQ ID NO: 4;
(iii) a polynucleotide sequence encoding the HBV PreS2.S antigen consisting of the sequence of SEQ ID NO: 6;
(iv) a polynucleotide sequence encoding the HBV core antigen consisting of the sequence of any one of SEQ ID NO: 87, SEQ ID NO: 88 or SEQ ID NO: 89; and
(v) a polynucleotide sequence encoding the HBV pol antigen consisting of the sequence of SEQ ID NO: 10;
where the polynucleotide sequence encoding each of the first and second HBV Pre-S1 antigens, the HBV core antigen and the HBV pol antigen is independently operably linked to a polynucleotide encoding a signal peptide, such as the polynucleotide comprising the sequence of SEQ ID NO: 90.

12. The nucleic acid combination of claim 1, wherein each of the first, second and third autoprotease peptides independently comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof.

13. The nucleic acid combination of claim 1, wherein each of the first, second and third IRES is derived from encephalomyocarditis virus (EMCV) or Enterovirus 71 (EV71).

14. The nucleic acid combination of claim 1, comprising, ordered from the 5′- to 3′-end:
(1) a polynucleotide sequence encoding an HBV Pre-S1 antigen having the amino acid sequence of SEQ ID NO: 1 or 3, a polynucleotide sequence encoding a P2A amino acid sequence of SEQ ID NO: 11 or an IRES having the polynucleotide sequence of SEQ ID NO: 13 or 14, and a polynucleotide sequence encoding an HBV PreS2.S antigen having the amino acid sequence of SEQ ID NO: 5;
(2) a polynucleotide sequence encoding an HBV PreS2.S antigen having the amino acid sequence of SEQ ID NO: 5, a polynucleotide sequence encoding a P2A amino acid sequence of SEQ ID NO: 11 or an IRES having the polynucleotide sequence of SEQ ID NO: 13 or 14, and a polynucleotide sequence encoding an HBV Pre-S1 antigen having the amino acid sequence of SEQ ID NO: 1 or 3;
(3) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(4) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(5) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(6) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(7) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(8) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 13, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(9) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(10) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 13, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(11) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(12) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 14, a polynucleotide sequence encoding an HBV PreS2.S antigen having the amino acid sequence of SEQ ID NO: 5, a polynucleotide sequence encoding a P2A amino acid sequence of SEQ ID NO: 11, and a polynucleotide sequence encoding an HBV Pre-S1 antigen having the amino acid sequence of SEQ ID NO: 1 or 3;
(13) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(14) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV Pre-S1 antigen having the amino acid sequence of SEQ ID NO: 1 or 3, an IRES having the polynucleotide sequence of SEQ ID NO: 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(15) a polynucleotide sequence encoding an HBV PreS2.S antigen having the amino acid sequence of SEQ ID NO: 5, an IRES having the polynucleotide sequence of SEQ ID NO: 13 or 14, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(16) a polynucleotide sequence encoding an HBV PreS2.S antigen having the amino acid sequence of SEQ ID NO: 5, an IRES having the polynucleotide sequence of SEQ ID NO: 14, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(17) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, an IRES having the polynucleotide sequence of SEQ ID NO: 13 or 14, and a polynucleotide sequence encoding an HBV PreS2.S antigen having the amino acid sequence of SEQ ID NO: 5;
(18) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, an IRES having the polynucleotide sequence of SEQ ID NO: 13 or 14, and a polynucleotide sequence encoding an HBV PreS2.S antigen having the amino acid sequence of SEQ ID NO: 5;
(19) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV core antigen having the amino acid sequence of any one of SEQ ID NOs: 84, 85, or 86;
(20) a polynucleotide sequence encoding an HBV PreS2.S antigen having the amino acid sequence of SEQ ID NO: 5, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV polymerase antigen having the amino acid sequence of SEQ ID NO: 9;
(21) a polynucleotide sequence encoding an HBV polymerase antigen having the amino acid sequence of SEQ ID NO: 9, a polynucleotide sequence encoding a P2A amino acid sequence of SEQ ID NO: 11, a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, and a polynucleotide sequence encoding an HBV PreS2.S antigen having the amino acid sequence of SEQ ID NO: 5; or
(22) a polynucleotide sequence encoding an HBV core antigen having the amino acid sequence of any one of SEQ ID NOs: 84, 85, or 86, a polynucleotide sequence encoding a P2A amino acid sequence of SEQ ID NO: 11, a polynucleotide sequence encoding an HBV polymerase antigen having the amino acid sequence of SEQ ID NO: 9, a polynucleotide sequence encoding a P2A amino acid sequence of SEQ ID NO: 11, and a polynucleotide sequence encoding an HBV PreS2.S antigen having the amino acid sequence of SEQ ID NO: 5.

15. The nucleic acid combination of claim 14, comprising the non-naturally occurring polynucleotide sequence of any one of SEQ ID NOs: 15 to 54.

16. A vector comprising the nucleic acid combination of claim 1.

17. The vector of claim 16 being a DNA plasmid.

18. The vector of claim 16 being a DNA viral vector or an RNA viral vector.

19. The vector of claim 18 being a Modified Vaccinia Ankara (MVA) vector or an adenovirus vector.

20. The vector of claim 19 being an Ad26, Ad35, or MVA-BN vector.

21. An RNA replicon, comprising, ordered from the 5′- to 3′-end:
(1) a 5′ untranslated region (5′-UTR) required for nonstructural protein-mediated amplification of an RNA virus;
(2) a polynucleotide sequence encoding at least one of non-structural proteins of the RNA virus;
(3) a subgenomic promoter of the RNA virus;
(4) the nucleic acid combination of claim 1; and
(5) a 3′ untranslated region (3′-UTR) required for nonstructural protein-mediated amplification of the RNA virus.

22. The RNA replicon of claim 21, comprising, ordered from the 5′- to 3′-end,
(1) an alphavirus 5′ untranslated region (5′-UTR);
(2) a 5′ replication sequence of an alphavirus non-structural gene nsp1;
(3) a downstream loop (DLP) motif of a virus species;
(4) a polynucleotide sequence encoding a fourth autoprotease peptide;
(5) a polynucleotide sequence encoding alphavirus non-structural proteins nsp1, nsp2, nsp3 and nsp4;
(6) an alphavirus subgenomic promoter;
(7) the nucleic acid combination of claim 1;
(8) an alphavirus 3′ untranslated region (3′ UTR); and
(9) optionally, a poly adenosine sequence.

23. The RNA replicon of claim 22, wherein the DLP motif is from a virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MTDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (U AV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus.

24. The RNA replicon of claim 23, wherein the fourth autoprotease peptide is selected from the group consisting of porcine tesehovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2 A (BmIFV2A), and a combination thereof.

25. The RNA replicon of claim 21, comprising, ordered from the 5′- to 3′-end,
(1) a 5′-UTR having the polynucleotide sequence of SEQ ID NO: 55,
(2) a 5′ replication sequence having the polynucleotide sequence of SEQ ID NO: 56,
(3) a DLP motif comprising the polynucleotide sequence of SEQ ID NO: 57,
(4) a polynucleotide sequence encoding a P2A sequence of SEQ ID NO: 11,
(5) polynucleotide sequences encoding alphavirus non-structural proteins nsp1, nsp2, nsp3 and nsp4, having the nucleic acid sequences of SEQ ID NO: 58, SEQ ID NO: 59, SEQ ID NO: 60 and SEQ ID NO: 61, respectively,
(6) a subgenomic promoter having polynucleotide sequence of SEQ ID NO: 62,
(7) the nucleic acid combination of claim 1, and
(8) a 3′ UTR having the polynucleotide sequence of SEQ ID NO: 63.

26. The RNA replicon of claim 25, wherein:
(i) the polynucleotide sequence encoding the P2A sequence comprises SEQ ID NO: 12,
(ii) the nucleic acid combination comprises the polynucleotide sequence of any one of SEQ ID NOs: 15 to 54, and
(iii) the RNA replicon further comprises a poly adenosine sequence at the 3′-end of the replicon.

27. An RNA replicon comprising the polynucleotide sequence of any one of SEQ ID NOs: 65 to 72.

28. A nucleic acid molecule comprising a polynucleotide sequence encoding the RNA replicon of claim 21.

29. A pharmaceutical composition comprising the nucleic acid combination of claim 1 and a pharmaceutically acceptable carrier.

30. The pharmaceutical composition of claim 29, wherein the pharmaceutically acceptable carrier comprises a lipid nanoparticle.

31. The pharmaceutical composition of claim 29, further comprising:
(1) a polynucleotide sequence encoding an HBV core antigen having the amino acid sequence of any one of SEQ ID NOs: 84, 85 or 86, a polynucleotide sequence encoding a P2A amino acid sequence of SEQ ID NO: 11 or an IRES having the polynucleotide sequence of SEQ ID NO: 13 or 14, and a polynucleotide sequence encoding an HBV polymerase antigen having the amino acid sequence of SEQ ID NO: 9; or
(2) a polynucleotide sequence encoding an HBV polymerase antigen having the amino acid sequence of SEQ ID NO: 9, a polynucleotide sequence encoding a P2A amino acid sequence of SEQ ID NO: 11 or an IRES having the polynucleotide sequence of SEQ ID NO: 13 or 14, and a polynucleotide sequence encoding an HBV core antigen having the amino acid sequence of any one of SEQ ID NOs: 84, 85 or 86.

32. A method of treating HBV infection in a subject in need thereof, comprising administering to the subject the pharmaceutical composition according to claim 29.

33. The method of claim 32, wherein the pharmaceutical composition is a therapeutic vaccine against HBV.

34. The method of claim 32, further comprising administering a second composition comprising a nucleic acid molecule encoding at least one identical HBV antigen for use as a prime-boost regimen.

35. The method of claim 34, wherein the prime-boost regimen comprises a first composition comprising the RNA replicon of claim 21 and a second composition comprising a vector which is not an RNA replicon and which encodes at least one identical HBV epitope as the first composition, and one of the first and second compositions is used for priming vaccination and the other is used for boosting vaccination.

36. The method of claim 35, wherein the second composition comprises a Modified Vaccinia Ankara (MVA) vector, an adenovirus vector or a plasmid vector.

37. The method of claim 36, wherein the second composition comprises an Ad26, Ad35, or MVA-BN vector.

38. A method of inducing an immune response against a hepatitis B virus (HBV) in a subject in need thereof, comprising administering to the subject the pharmaceutical composition of claim 28, optionally in combination with another immunogenic agent or other anti-HBV agent.

39. The method of claim 38, wherein administration in combination with another immunogenic agent or other anti-HBV agent is not optional, and the other anti-HBV agent is a small molecule, an antibody or antigen binding fragment thereof, a polypeptide, protein, or nucleic acid.

40. A method of reducing infection and/or replication of HBV in a subject in need thereof, comprising administering to the subject a pharmaceutical composition according to claim 29.

41. An isolated host cell comprising the nucleic acid combination of claim 1.

42. A method of producing an RNA replicon, comprising transcribing the nucleic acid according to claim 28 in vivo or in vitro.
